(19) United States
(12) Patent Application Publication
(10) Pub. No.: US 2007/0065902 A1
(43) Pub. Date: Mar. 22, 2007

(54) Process for chromosomal expression of foreign genes in the fliC region of a methylotrophic microbial host cell

(76) Inventors: Deana J. Dicosimo, Rockland, DE (US); Pamela L. Sharpe, Newark, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/230,161

(22) Filed: Sep. 19, 2005

(51) Int. Cl. C12P 23/00; C07H 21/04; C12N 9/10; C12N 15/74; C12N 1/21

(52) U.S. Cl. 435/67; 435/252.3; 435/471; 435/193; 536/23.2

(57) ABSTRACT

Provided is a method for expressing an introduced gene or genes in a methylotrophic microorganism host wherein the gene(s) are integrated into the fliC region of the chromosome. This method provides stable, high-level expression of the integrated genes in which growth rate of the host strain is not adversely affected and a selection marker is not required. The use of this method for expressing carotenoid biosynthetic genes and resulting production of astaxanthin is also described.

### FIELD OF INVENTION

The present invention relates to bacterial gene expression and metabolic engineering. More specifically, this invention relates to a method for the stable expression of introduced genes in the fliC chromosomal region of a methylotrophic microorganism.

### BACKGROUND OF THE INVENTION

There are a number of microorganisms that utilize single carbon substrates as their sole source of carbon and energy. Such microorganisms are referred to herein as “C₁ metabolizers”. All C₁ metabolizing microorganisms are generally classified as methylotrophs. Methylotrophs may be defined as any organism capable of oxidizing organic compounds that do not contain carbon-carbon bonds, such as methane and/or methanol. Methanotrophic bacteria are a class of methylotrophic bacteria defined by their ability to use methane as their sole source of carbon and energy under ambient conditions. This ability, in conjunction with the abundance of methane, makes the biotransformation of methane a potentially unique and valuable process.

Odom et al. have investigated Methylomonas sp. 16a as a microbial platform of choice for production of a variety of materials including carbohydrates, pigments, terpenoid compounds and aromatic compounds (U.S. Pat. No. 6,537,786, U.S. Pat. No. 6,689,601, U.S. Pat. No. 6,660,507, U.S. Pat. No. 6,818,424, and U.S. Ser. No. 09/941,947). This particular methanotrophic bacterial strain is capable of efficiently using methanol and/or methane as a carbon substrate, is metabolically versatile in that it contains multiple pathways for the incorporation of carbon from formaldehyde into 3-carbon units, and is amenable to genetic engineering via bacterial conjugation using donor species such as Escherichia coli (U.S. Ser. No. 10/997,308 and U.S. Ser. No. 10/997,844). Thus, Methylomonas sp. 16a can be engineered to produce new classes of products other than those naturally produced from methane.

Microbial production of industrial compounds requires the ability to efficiently engineer changes to the genome of an organism. Engineering changes such as adding, removing, or modifying genetic elements has often proven to be challenging and time consuming exercises. One such modification is genetically engineering modulations to the expression of relevant genes in a metabolic pathway.

There are a variety of ways to modulate gene expression. Microbial metabolic engineering frequently involves the use of multi-copy vectors to express a gene of interest under the control of a strong constitutive or conditional promoter. Plasmid-based expression systems facilitate the ability to express multiple copies of the same gene within the transformed host cell. However, maintenance of the plasmid within the host normally requires selective pressure. This is typically accomplished by using a plasmid expressing an antibiotic resistance marker. Nutritional selection markers may also be used, but these generally decrease the growth rate of the host cell.

Commercial fermentative production is best achieved when no selective pressure is required to maintain the presence of the introduced gene(s). The presence of an antibiotic resistance gene is undesirable in terms of both cost and required regulatory approvals. Thus, there is a need to express and maintain the introduced gene(s) in the recombinant host cell without the use of antibiotic resistance. Additionally, the metabolic burden of maintaining a vector normally decreases the overall growth rate of the host cell. As such, the use of a vector-based expression systems has characteristics that are undesirable for certain commercial production applications. Chromosomal expression can be used to circumvent the detrimental growth effects associated with vector burden and the need for selective pressure. Suitable integration sites need to be identified that facilitate stable expression of the introduced DNA at levels adequate for industrial production of the desired end product. The insertion of foreign DNA into the chosen integration site must not be detrimental to the host cell's survival, genetic stability, and/or growth rate. Accordingly, there is a need to identify suitable integration sites within the host cell's genome.

A previous method to identify suitable chromosomal integration sites within a C₁-metabolizing host cell (Methylomonas sp. 16a) has been described, resulting in the identification of the tig region (Miller, E. and Ye, R., U.S. Ser. No. 11/070,080; hereby incorporated by reference). However, microbial metabolic pathway engineering typically requires a plurality of genetic modifications to optimally produce the desired product at commercially useful levels. Hence, the identification of additional integration sites suitable for expressing introduced genes at levels sufficient to produce the desired product are needed. The problem to be solved is to identify suitable chromosomal integration sites within a methylotrophic bacteria for recombinant gene expression that exhibit significant transcriptional activity and/or genetic stability. Insertion of DNA within the selected region should not result in significant adverse effects to the host cell's survival or growth rate.

### SUMMARY OF THE INVENTION

The stated problem has been solved by identifying the fliC chromosomal region in a methylotrophic bacterial host cell a optimal for integration for the expression of foreing genes. Transformed host cells comprising an insertion in the fliC region exhibited high level expression of a promoterless reporter construct (carotenoid biosynthesis gene cluster) when operably linked to the endogenous fliC promoter. In addition, recombinant host cells comprising the chromosomally-integrated DNA stably expressed the introduced genes over several generations without any significant detrimental effects on viability or growth rate.

Accordingly, a method for stably expressing a nucleic acid molecule in a methylotrophic microorganism is provided comprising:
a) providing a methylotrophic microorganism having a fliC genomic region;
b) providing at least one expressible nucleic acid molecule to be stably expressed;
c) integrating the at least one expressible nucleic acid molecule of (b) into said fliC region of the genome of said methylotrophic microorganism whereby a transformed methylotrophic microorganism is created; and
d) growing the transformed methylotrophic methylotrophic microorganism of c) under suitable conditions whereby said at least one nucleic acid molecule is stably expressed.

The reporter gene used to identify suitable integration sites was a promoterless carotenoid gene cluster encoding enzymes responsible for astaxanthin biosynthesis. Operably linking the promoterless construct to the fliC promoter resulted in the production of the carotenoid pigment. In another aspect, a method for the production of a carotenoid compound in a methylotrophic host cell is provided comprising:

a) providing a methylotrophic microorganism comprising at least one expressible nucleic acid molecule encoding at least one carotenoid biosynthetic pathway enzyme chromosomally integrated into a fliC genomic region of said microorganism;

b) contacting the methylotrophic microorganism with a C₁ carbon substrate selected from the group consisting of methane and methanol under conditions whereby said at least one expressible nucleic acid molecule is expressed and at least one carotenoid compound is produced; and

c) optionally isolating the carotenoid of step b).

The promoterless carotenoid biosynthesis gene cluster chromosomally integrated and operably linked to the fliC promoter was highly expressed, resulting in the production of the carotenoid compound at levels similar to those observed in multicopy plasmid-based expression systems. In another aspect, an isolated nucleic acid fragment encoding the fliC promoter is provided as represented by SEQ ID NO: 34.

In a further aspect, a method for expressing a coding region of interest in a methylotrophic bacteria is provided comprising;
a) providing a methylotrophic bacteria comprising a chimeric gene, said chimeric gene comprising a fliC promoter operably linked to a coding region of interest expressible in a methylotrophic bacteria; and
b) growing said methylotrophic bacteria under suitable growth conditions whereby said chimeric gene is expressed.

Although the present invention is exemplified by the integration and expression of carotenoid biosynthesis genes, the skilled artisan will recognize that the fliC region will be useful for the insertion of other foreign genes.

In another aspect, the invention provides a methylotrophic microorganism comprising at least one nucleic acid molecule integrated in the fliC genomic region.

### BRIEF DESCRIPTION OF THE FIGURES, SEQUENCE DESCRIPTIONS, AND BIOLOGICAL DEPOSITS

FIG. 1 shows the upper carotenoid and lower carotenoid biosynthetic pathways.

FIG. 2 shows a plasmid map of the pUTmTn5 vector comprising a multiple cloning site (MCS).

FIG. 3 shows a plasmid map of the pUTmTn5 Cm vector.

FIG. 4 shows the design of the promoterless transposon construct used to identify suitable integration sites with the methylotrophic host cell genome. Regions amplified by sequencing primers A through J are shown.

FIG. 5 shows the genetic arrangement of the fliC region of the Methylomonas genome and the integration sites identified by screening.

The invention can be more fully understood from the following detailed description and the accompanying sequence descriptions, which form a part of this application.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the European Patent Convention (EPC) and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NO:1 is the nucleotide sequence of carotenoid biosynthesis plasmid pDCQ334.

SEQ ID NO:2 is the nucleotide sequence of carotenoid biosynthesis plasmid pDCQ341.

SEQ ID NO:3 is the nucleotide sequence of carotenoid biosynthesis plasmid pDCQ343.

SEQ ID NO:4 is the nucleotide sequence of carotenoid biosynthesis plasmid pDCQ377.

SEQ ID NO: 5 is the nucleotide sequence of the carotenoid gene cluster crtWZEidiYIB in plasmid pDCQ334.

SEQ ID NO: 6 is the nucleotide sequence of the crtWEYIB gene cluster in plasmid pDCQ341.

SEQ ID NO: 7 is the nucleotide sequence of the crtWZEYIB gene cluster in plasmid pDCQ343.

SEQ ID NO: 8 is the nucleotide sequence of the crtWZEidiYIB gene cluster in plasmid pDCQ377.

SEQ ID NO: 9 is the nucleotide sequence of the primer MCS.F.

SEQ ID NO: 10 is the nucleotide sequence of the primer MCS.R.

SEQ ID NO: 11 is the nucleotide sequence of the primer pUTmTn5/Seq.F.

SEQ ID NO:12 is the nucleotide sequence of the primer pUTmTn5/Seq. R.

SEQ ID NO: 13 is the nucleotide sequence of the primer KnavrIIKpnIBstBI.R2.

SEQ ID NO: 14 is the nucleotide sequence of the primer KnBstBI.F.

SEQ ID NO: 15 is the nucleotide sequence of the Sphingomonas melonis DC18 crtW ketolase coding region in pDCQ343.

SEQ ID NO: 16 is the nucleotide sequence of the Brevundimonas vesicularis DC263 crtZ hydroxylase coding region in pDCQ343.

SEQ ID NO: 17 is the nucleotide sequence of primer p343crtZSpeI.F.

SEQ ID NO: 18 is the nucleotide sequence of primer p343crtWSpeI.R

SEQ ID NO: 19 is the nucleotide sequence of primer CmAvrIIKpnIBstBI.R.

SEQ ID NO: 20 is the nucleotide sequence of primer CmBstBI.F.

SEQ ID NO: 21 is the nucleotide sequence of primer crtE343R.

SEQ ID NO: 22 is the nucleotide sequence of primer pUTmTn5-334KnPCR.F.

SEQ ID NO: 23 is the nucleotide sequence of primer pUTmTn5-334KnPCR.R.

SEQ ID NO: 24 is the nucleotide sequence of primer pUTmTn5-334KnSeq.F.

SEQ ID NO: 25 is the nucleotide sequence of primer pUTmTn5-334KnSeq.R.

SEQ ID NO: 26 is the nucleotide sequence of primer pUTmTn5-343CmPCR.F.

SEQ ID NO: 27 is the nucleotide sequence of primer pUTmTn5-343CmSeq.F.

SEQ ID NO: 28 is the nucleotide sequence of primer pUTmTn5-343CmPCR.R.

SEQ ID NO: 29 is the nucleotide sequence of primer pUTmTn5-343CmSeq.R.

SEQ ID NO: 30 is the nucleotide sequence of primer pUTmTn5-377KnPCR.F.

SEQ ID NO: 31 is the nucleotide sequence of primer pUTmTn5-377KnSeq.F.

SEQ ID NO: 32 is the nucleotide sequence of the chloramphenicol resistance gene amplified from pUTmTn5Cm.

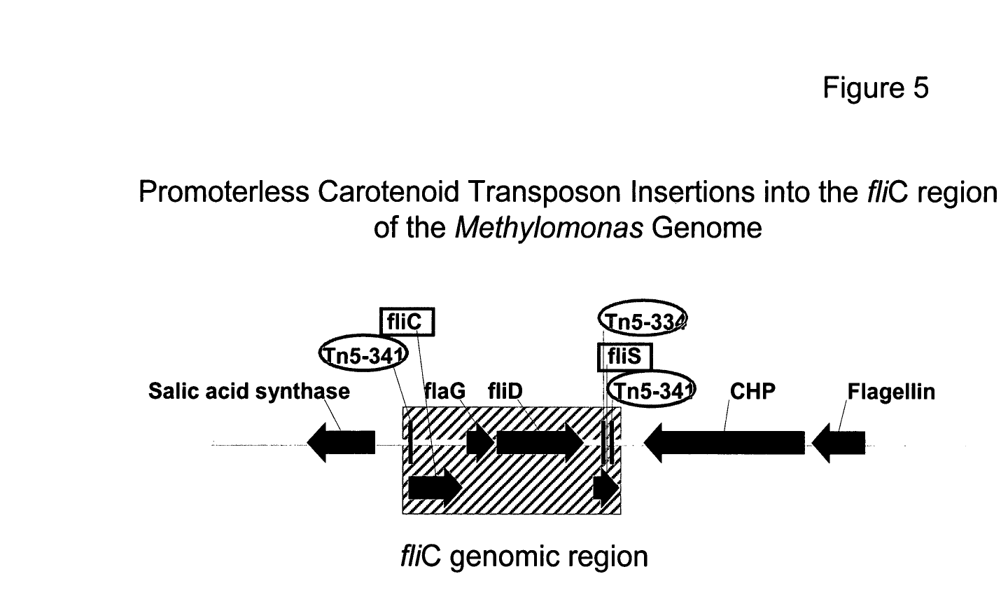

SEQ ID NO: 33 is the nucleotide sequence of the fliC region identified in Methylomonas sp. 16a (ATCC PTA-2402). The fliC region in Methylomonas sp. 16a is comprised of 4 open reading frames identified as fliC, flaG, fliD, and fliS (FIG. 5).

SEQ ID NO: 34 is the nucleotide sequence of the fliC promoter.

SEQ ID NO: 35 is the nucleotide sequence of the fliC open reading frame.

SEQ ID NO: 36 is the deduced amino acid sequence of encoded by the fliC open reading frame.

SEQ ID NO: 37 is the nucleotide sequence of the flaG open reading frame.

SEQ ID NO: 38 is the deduced amino acid sequence of encoded by the flaG open reading frame.

SEQ ID NO: 39 is the nucleotide sequence of the fliD open reading frame.

SEQ ID NO: 40 is the deduced amino acid sequence of encoded by the fliD open reading frame.

SEQ ID NO: 41 is the nucleotide sequence of the fliS open reading frame.

SEQ ID NO: 42 is the deduced amino acid sequence of encoded by the fliS open reading frame.

SEQ ID NO: 43 is the 16s rRNA gene sequence from Methylomonas sp. 16a (ATCC PTA-2402) and derivatives thereof such as Methylomonas sp. MWM1200 (ATCC PTA-6887).

The following biological deposits were made under the terms of the Budapest Treaty on the International Recognition of the Deposit of Microorganisms for the Purposes of Patent Procedure:

| | International | |
|---|---|---|
| Depositor Identification | Depository | |
| Reference | Designation | Date of Deposit |
| Methylomonas 16a | ATCC PTA-2402 | Aug. 22 2000 |
| Methylomonas sp. MWM1200 | ATCC PTA-6887 | Jul. 22, 2005 |

As used herein, “ATCC” refers to the American Type Culture Collection International Depository Authority located at ATCC, 10801 University Blvd., Manassas, Va. 20110-2209, USA. The “International Depository Designation” is the accession number to the culture on deposit with ATCC.

The listed deposit will be maintained in the indicated international depository for at least thirty (30) years and will be made available to the public upon the grant of a patent disclosing it. The availability of a deposit does not constitute a license to practice the subject invention in derogation of patent rights granted by government action.

### DETAILED DESCRIPTION OF THE INVENTION

The present invention relates to the finding that the fliC region of the genome of a methylotrophic microorganism is a suitable location for the integration and expression of foreign genes. In particular, it has been discovered that a gene cluster encoding the enzymes of the lower carotenoid pathway, when inserted into this region, stably produced high levels of C₄₀ carotenoids (e.g. astaxanthin).

In another aspect, the fliC region is used for the recombinant expression of the carotenoid biosynthesis genes in methylotrophic bacteria. In a further aspect, the methylotrophic bacteria is the methanotrophic bacteria Methylomonas sp. 16a (ATCC PTA-2402) and derivatives thereof.

In yet a further aspect, a nucleic acid sequence encoding the fliC promoter is provided. In yet another aspect, a method for recombinantly expressing a chimeric gene comprised of the fliC promoter is also provided.

Definitions

In this disclosure, a number of terms and abbreviations are used. The following definitions are provided:

As used herein, the term “open reading frame” is abbreviated ORF.

“Polymerase chain reaction” is abbreviated PCR.

“High Performance Liquid Chromatography” is abbreviated HPLC.

As used herein, “kanamycin” is abbreviated Kan.

As used herein, “ampicillin” is abbreviated Amp.

As used herein, the term “methylotroph” means a microorganism capable of oxidizing organic compounds that do not contain carbon-carbon bonds. Methylotrophs having the ability to oxidize methane (CH₄) are further characterized as methanotrophs. In one embodiment, the methylotroph utilizes methanol and/or methane as a primary carbon source.

As used herein, the term “methanotroph” or “methanotrophic bacteria” means a prokaryote capable of utilizing methane as its primary source of carbon and energy. Complete oxidation of methane to carbon dioxide occurs by aerobic degradation pathways. Typical examples of methanotrophs useful in the present invention include (but are not limited to) the genera Methylomonas, Methylobacter, Methylococcus, and Methylosinus. In one embodiment, the methanotrophic bacterium is a high growth methanotrophic bacteria comprising a functional Embden-Meyerhof carbon flux pathway (U.S. Pat No. 6,689,601). In another embodiment, the high growth methanotrophic bacterium is Methylomonas sp. 16a (ATCC PTA-2402) and mutant derivatives thereof. In one aspect, the term “mutant derivatives” or “derivatives of Methylomonas sp. 16a” refers to Methylomonas strains developed from Methylomonas sp. 16a (ATCC PTA-2402). In a further aspect, the mutant derivatives of Methylomonas sp. 16a are comprised of the 16s rRNA gene sequence as represented by SEQ ID NO: 43 (U.S. Pat No. 6,689,601; hereby incorporated by reference). In yet another embodiment, the methanotroph utilizes methanol and/or methane as a primary carbon source.

As used herein, the term “pigmentless” or “white mutant” refers to a Methylomonas sp. 16a bacterium wherein the native pink pigment (e.g., a C₃₀ carotenoid) is not produced (U.S. Ser. No. 10/997,844, hereby incorporated by reference). Expression of several genes involved in C₃₀ carotenoid production were disrupted (i.e. crtN1, ald, crtN2), thereby creating a pigmentless mutant (e.g. Methylomonas MWM1200). Thus, the bacterial cells appear white in color, as opposed to pink.

As used herein, the term “MWM1200 (Δcrt cluster promoter+ΔcrtN3)” or “MWM1200” refers to a mutant of Methylomonas sp. 16a (ATCC PTA-2402) in which the endogenous C₃₀ carotenoid gene cluster promoter and the crtN3 gene have been disrupted. Methylomonas sp. MWM1200 has been deposited to the American Type Culture Collection under accession number PTA-6887. Disruption of the native C₃₀ carotenoid biosynthetic pathway resulted in a suitable background (pigmentless) for engineering C₄₀ carotenoid production (U.S. Ser. No. 10/997,844; hereby incorporated by reference).

As used herein, the terms “fliC region” and “fliC genomic region” refer to the region of chromosomal DNA containing coding regions that are all expressed from the fliC promoter. The fliC region includes the coding region for the flagellin subunit protein, as well as any other adjacent coding regions that do not have promoters, but are transcribed together with the flagellin subunit protein coding region. The Methylomonas sp. 16a fliC region is comprised of at least 4 open reading frames operably linked to the fliC promoter including fliC, flaG, fliD, and fliS (FIG. 5). In one aspect, the fliC region is comprised of the coding regions having the following organization: fliC-flaG-fliD-fliS. In another aspect, the fliC region is represented by SEQ ID NO: 33. Foreign genes and/or nucleic acid molecules comprised of one or more coding sequences can be inserted and stably expressed anywhere within the fliC region. In another aspect, the insertion site is selected from the group consisting of coding region for fliC (SEQ ID NO: 35), flaG (SEQ ID NO: 37), fliD (SEQ ID NO: 39), and fliS (SEQ ID NO: 41). In yet another aspect, the insertion site is selected from the group consisting of fliC (SEQ ID NO: 35) and fliS (SEQ ID NO: 41).

As used herein, the term “fliC promoter” refers to the DNA sequence located 5′ to the coding region for the flagellin subunit protein that directs transcription of at least this coding region. The fliC promoter is represented by SEQ ID NO: 34.

As used herein, the term “fliC gene” refers to a gene encoding the flagellin subunit protein. Flagellin molecules are found in a variety of diverse bacterial species and share conserved amino acid residues, making it possible to identify flagellin gene by sequence similarity (Millikan et al., Appl. Environ. Microbiol. 65(7):3129-3133 (1999)). The coding sequence for the fliC gene is represented by SEQ ID NO: 35 encoding the amino acid represented by SEQ ID NO: 36.

As used herein, the term “flaG gene” refers to a gene encoding a protein involved in forming an export apparatus for flagellar proteins. The coding sequence for the flaG gene is represented by SEQ ID NO: 37 encoding the amino acid sequence represented by SEQ ID NO: 38.

As used herein, the term “fliD gene” refers to a gene encoding the flagellar hook-associated capping protein. The coding sequence for the fliD gene is represented by SEQ ID NO: 39 encoding the amino acid sequence represented by SEQ ID NO: 40.

As used herein, the term “fliS gene” refers to a gene encoding a flagellar protein acting as a flagellin specific chaperone. The coding sequence for the fliS gene is represented by SEQ ID NO: 41 encoding the amino acid sequence represented by SEQ ID NO: 42.

As used herein, the term “bacterial flagellum” refers to an organelle used for motility and is comprised of protein subunits termed flagellin.

As used herein, the term “isoprenoid compound” refers to compounds formally derived from isoprene (2-methylbuta-1,3-diene; CH₂═C(CH₃)CH═CH₂), the skeleton of which can generally be discerned in repeated occurrence in the molecule. These compounds are produced biosynthetically via the isoprenoid pathway beginning with isopentenyl pyrophosphate (IPP) and formed by the head-to-tail condensation of isoprene units, leading to molecules which may be, for example, of 5, 10, 15, 20, 30, or 40 carbons in length.

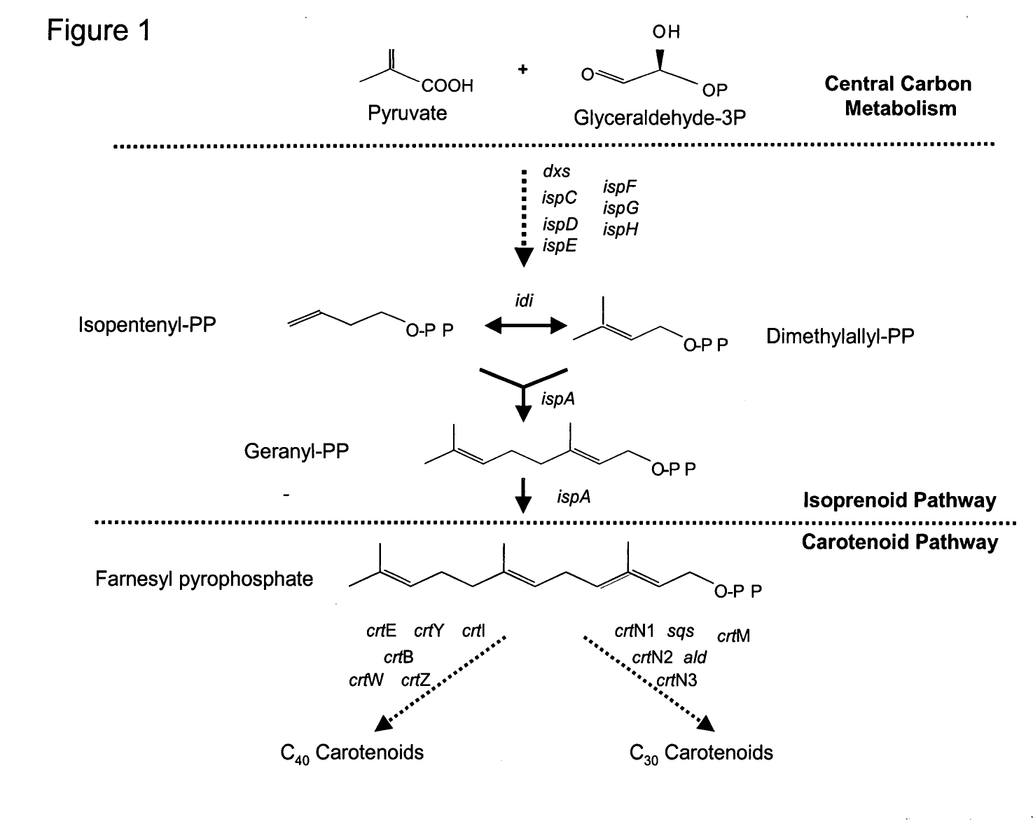

As used herein, the term “carotenoid biosynthetic pathway” or refers to those genes comprising members of the upper carotenoid pathway and/or lower carotenoid biosynthetic pathway, as illustrated in FIG. 1.

As used herein, the terms “upper carotenoid pathway” and “upper pathway” are used interchangeably and refer to enzymes involved in converting pyruvate and glyceraldehyde-3-phosphate to farnesyl pyrophosphate (FPP). Genes encoding these enzymes include, but are not limited to: the “dxs” gene (encoding 1-deoxyxylulose-5-phosphate synthase); the “dxr” gene (encoding 1-deoxyxylulose-5-phosphate reductoisomerase); the “ispD” gene (encoding a 2C-methyl-D-erythritol cytidyltransferase enzyme; also known as ygbP); the “ispE” gene (encoding 4-diphosphocytidyl-2-C-methylerythritol kinase; also known as ychB); the “ispF” gene (encoding a 2C-methyl-D-erythritol 2,4-cyclodiphosphate synthase; also known as ygbB); the “pyrG” gene (encoding a CTP synthase); the “lytB” gene involved in the formation of dimethylallyl diphosphate; the “gcpE” gene involved in the synthesis of 2-C-methyl-D-erythritol 4-phosphate; the “idi” gene (responsible for the intramolecular conversion of IPP to dimethylallyl pyrophosphate); and the “ispA” gene (encoding geranyltransferase or farnesyl diphosphate synthase) in the isoprenoid.

As used herein, the terms “lower carotenoid biosynthetic pathway” and “lower pathway” will be used interchangeably and refer to those enzymes which convert FPP to a suite of carotenoids. These include those genes and gene products that are involved in the immediate synthesis of either diapophytoene (whose synthesis represents the first step unique to biosynthesis of C₃₀ carotenoids) or phytoene (whose synthesis represents the first step unique to biosynthesis of C₄₀ carotenoids). All subsequent reactions leading to the production of various C₃₀-C₄₀ carotenoids are included within the lower carotenoid biosynthetic pathway. These genes and gene products comprise all of the “crt” genes including, but not limited to: crtM, crtN1, crtN2, crtE, crtX, crtY, crtI, crtB, crtZ, crtW, crtR, crtL, crtO, crtA, crtC, crtD, crtF, and crtU. Finally, the term “lower carotenoid biosynthetic enzyme” is an inclusive term referring to any and all of the enzymes in the present lower pathway including, but not limited to: CrtM, CrtN, CrtN2, CrtE, CrtX, CrtY, CrtI, CrtB, CrtZ, CrtW, CrtR, CrtL, CrtO, CrtA, CrtC, CrtD, CrtF, and CrtU.

As used herein, the term “carotenoid” refers to a class of hydrocarbons having a conjugated polyene carbon skeleton formally derived from isoprene. This class of molecules is composed of C₃₀ diapocarotenoids and C₄₀ carotenoids and their oxygenated derivatives; and, these molecules typically have strong light absorbing properties. The oxygenated derivatives are commonly referred to as “xanthophylls”.

As used herein, the term “tetraterpenes” or “C₄₀ carotenoids” refers to carotenoid compounds consisting of eight isoprenoid units joined in such a manner that the arrangement of isoprenoid units is reversed at the center of the molecule so that the two central methyl groups are in a 1,6-positional relationship and the remaining non-terminal methyl groups are in a 1,5-positional relationship. All C₄₀ carotenoids may be formally derived from the acyclic C₄₀H₅₆ structure. Non-limiting examples of C₄₀ carotenoids include: phytoene, lycopene, β-carotene, zeaxanthin, astaxanthin, and canthaxanthin.

As used herein, the term “CrtE” refers to a geranylgeranyl pyrophosphate synthase enzyme encoded by the crtE gene and which converts trans-trans-farnesyl diphosphate and isopentenyl diphosphate to pyrophosphate and geranylgeranyl diphosphate.

As used herein, the term “Idi” refers to an isopentenyl diphosphate isomerase enzyme (E.C. 5.3.3.2) encoded by the idi gene.

As used herein, the term “CrtY” refers to a lycopene cyclase enzyme encoded by the crtY gene which converts lycopene to β-carotene.

As used herein, the term “CrtI” refers to a phytoene desaturase enzyme encoded by the crtI gene. CrtI converts phytoene into lycopene via the intermediaries of phytofluene, ζ-carotene and neurosporene by the introduction of 4 double bonds.

As used herein, the term “CrtB” refers to a phytoene synthase enzyme encoded by the crtB gene which catalyzes the reaction from prephytoene diphosphate to phytoene.

As used herein, the term “CrtZ” refers to a carotenoid hydroxylase enzyme (e.g. β-carotene hydroxylase) encoded by the crtZ gene which catalyzes a hydroxylation reaction. The oxidation reaction adds a hydroxyl group to cyclic carotenoids having a β-ionone type ring. This reaction converts cyclic carotenoids, such as β-carotene or canthaxanthin, into the hydroxylated carotenoids zeaxanthin or astaxanthin, respectively. Intermediates in the process typically include β-cryptoxanthin and adonirubin. It is known that CrtZ hydroxylases typically exhibit substrate flexibility, enabling production of a variety of hydroxylated carotenoids depending upon the available substrates.

As used herein, the term “CrtW” refers to a carotenoid ketolase enzyme encoded by the crtW gene that catalyzes an oxidation reaction where a keto group is introduced on the β-ionone type ring of cyclic carotenoids. The term “carotenoid ketolase” or “ketolase” refers to the group of enzymes that can add keto groups to the ionone type ring of cyclic carotenoids.

As used herein, the term “CrtX” refers to a zeaxanthin glucosyl transferase enzyme encoded by the crt gene and which converts zeaxanthin to zeaxanthin-β-diglucoside.

As used herein, the term “crt gene cluster” refers to a tandemly-arrayed group of genes that encode proteins involved in carotenoid biosynthesis. All of the genes in a gene cluster are transcribed from the same promoter.

As used herein, the term “C₁ carbon substrate” refers to any carbon-containing molecule that lacks a carbon-carbon bond. Non-limiting examples are methane, methanol, formaldehyde, formic acid, formate, methylated amines (e.g., mono-, di-, and tri-methyl amine), methylated thiols, and carbon dioxide. In a preferred embodiment, the preferred C₁ carbon substrate is methanol and/or methane.

As used herein, the term “C₁ metabolizer” refers to a microorganism that has the ability to use a single carbon substrate as its sole source of energy and biomass. C₁ metabolizers will typically be methylotrophs and/or methanotrophs.

As used herein, the term “C₁ metabolizing bacteria” or “C₁ metabolizing microorganism” refers to bacteria that have the ability to use a single carbon substrate as their sole source of energy and biomass. C₁ metabolizing bacteria, a subset of C₁ metabolizers, will typically be methylotrophs and/or methanotrophs.

As used herein, the term “chromosomal integration” means that a DNA segment introduced into the cell becomes congruent with the chromosome of a microorganism through recombination between homologous DNA regions on the introduced DNA segment and within the chromosome. In another aspect, DNA can be chromosomally integrated using random transposition. As described herein, transposition was used to identify suitable chromosomal integration sites within the methylotrophic bacteria's genome. Once identified and sequenced, one of skill in the ask can designed DNA molecules for targeted chromosomal integration using homologous recombination.

As used herein, the term “operably inserted” means that the gene or genes that are integrated into a chromosomal region are organized in a manner in which the encoded proteins are expressed from those genes, and the proteins are functional. In general, operable insertion requires that the integrated gene be in the same orientation as any other genes in the same operon. As used herein, the term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

As used herein, the term “marker” means a gene that confers a phenotypic trait that is easily detectable through screening or selection. A marker used in screening is, for example, one whose conferred trait can be visualized. Genes involved in carotenoid production or that encode proteins (i.e. beta-galactosidase, beta-glucuronidase) that convert a colorless compound into a colored compound are examples of this type of marker. A screening marker gene may also be referred to as a reporter gene. A selectable marker is one wherein cells having the marker gene can be distinguished based on growth. For example, an antibiotic resistance marker serves as a useful selectable marker, since it enables detection of cells which are resistant to the antibiotic, when cells are grown on media containing that particular antibiotic.

A “nucleic acid” is a polymeric compound comprised of covalently linked subunits called nucleotides. Nucleic acids include polyribonucleic acid (RNA) and polydeoxyribonucleic acid (DNA), both of which may be single-stranded or double-stranded. DNA includes cDNA, genomic DNA, synthetic DNA, and semi-synthetic DNA.

As used herein, an “isolated nucleic acid molecule” or “fragment” is a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

A nucleic acid fragment is “hybridizable” to another nucleic acid fragment, such as a cDNA, genomic DNA, or RNA, when a single stranded form of the nucleic acid fragment can anneal to the other nucleic acid fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, 2^{nd} ed., Cold Spring Harbor Laboratory: Cold Spring Harbor (1989), particularly Chapter 11 and Table 11.1.

As used herein, the term “gene” refers to a nucleic acid fragment that expresses a specific protein. As defined herein, it may or may not include regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

As used herein, a gene that is “expressible” is one that produces a functional protein product.

As used herein, “synthetic genes” can be assembled from oligonucleotide building blocks that are chemically synthesized using procedures known to those skilled in the art. These building blocks are ligated and annealed to form gene segments that are then enzymatically assembled to construct the entire gene. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

As used herein, the term “homolog” or “homologue”, as applied to a gene, means any gene derived from the same or a different microbe having the same or similar function. In one embodiment, the homologous gene has nucleotide sequence similarity and function.

As used herein, the term “coding sequence” or “coding region of interest” refers to a DNA sequence that encodes a specific amino acid sequence. The present examples illustrate the use of a promoterless gene cluster comprised of several coding regions whose expression is controlled by chromosomally integrating the cluster near an endogenous promoter. In this way, the promoterless gene cluster is operably linked to the endogenous promoter.

As used herein, the term “codon optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide for which the DNA codes. Within the context of the present examples, several genes and DNA coding regions were codon optimized for optimal expression in Methylomonas sp.16a (i.e. crtWZ coding regions in pDCQ334).

As used herein, the term “suitable regulatory sequences” refers to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing sites, effector binding sites and stem-loop structures. In one aspect, a suitable regulatory sequence is the fliC promoter.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene at different stages of development, or in response to different environmental or physiological conditions. Promoters that cause a gene to be expressed in most cells at most times are commonly referred to as “constitutive promoters”. It is further recognized that since in most cases the exact boundaries of regulatory sequences have not been completely defined, DNA fragments of different lengths may have identical promoter activity. As described herein, the fliC promoter is a region of DNA capable of controlling expression of the genes within the fliC region. In a further aspect, the fliC promoter is operably linked to a coding region of interest for chimeric gene expression. In one aspect, the fliC promoter is a nucleic acid sequence having at least 95% identity to SEQ ID NO: 34. In a further aspect, the fliC promoter is a nucleic acid sequence as represented by SEQ ID NO: 34.

The “3′ non-coding sequences” refer to DNA sequences located downstream of a coding sequence encoding regulatory signals capable of affecting mRNA processing or gene expression.

As used herein, the term “transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

As used herein, the term “conjugation” refers to a particular type of transformation in which a unidirectional transfer of DNA (e.g., from a bacterial plasmid) occurs from one bacterium cell (i.e., the “donor”) to another (i.e., the “recipient”). The process involves direct cell-to-cell contact.

The terms “plasmid” and “vector” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a gene or genes into a cell. “Transformation vector” refers to a specific plasmid containing a foreign gene and having elements (in addition to the foreign gene) that facilitate transformation of a particular host cell.

As used herein, the term “sequence analysis software” refers to any computer algorithm or software program that is useful for the analysis of nucleotide or amino acid sequences. “Sequence analysis software” may be commercially available or independently developed. Typical sequence analysis software will include, but is not limited to: the GCG suite of programs (Wisconsin Package Version 9.0, Genetics Computer Group (GCG), Madison, Wis.); BLASTP, BLASTN, BLASTX (Altschul et al., J. Mol. Biol. 215:403-410 (1990)); DNASTAR (DNASTAR, Inc., Madison, Wis.); and the FASTA program incorporating the Smith-Waterman algorithm (W. R. Pearson, Comput. Methods Genome Res., [Proc. Int. Symp.], Meeting Date 1992, 111-20. Suhai, Sandor, Ed.; Plenum: New York, N.Y. (1994)). Within the context of this application it will be understood that where sequence analysis software is used for analysis, the results of the analysis are based on the “default values” of the program referenced, unless otherwise specified. As used herein “default values” will mean any set of values or parameters set by the manufacturer which originally load with the software when first initialized.

The invention relates to the integration of expressible nucleic acids of interest into the fliC chromosomal region of a methylotrophic microorganism. Preferred expressible nucleic acid molecules are those that comprise the carotenoid biosynthetic pathway. Integration of these genes at this specific point in the methylotrophic host genome results in stable expression of the integrated genes and robust carotenoid production.

Methylotrophic C1-Metabolizing Microorganism Host Cells

All C₁-metabolizing microorganisms are generally classified as methylotrophs. Methylotrophs may be defined as any organism capable of oxidizing organic compounds that do not contain carbon-carbon bonds. However, facultative methylotrophs, obligate methylotrophs, and obligate methanotrophs are all various subsets of methylotrophs. Specifically:
Facultative methylotrophs have the ability to oxidize organic compounds which do not contain carbon-carbon bonds, but may also use other carbon substrates such as sugars and complex carbohydrates for energy and biomass;
Obligate methylotrophs are those organisms which are limited to the use of organic compounds that do not contain carbon-carbon bonds for the generation of energy; and
Obligate methanotrophs are those obligate methylotrophs that have the distinct ability to oxidize methane.
   Facultative methylotrophic bacteria are found in many environments, but are isolated most commonly from soil, landfill and waste treatment sites. Many facultative methylotrophs are members of the β, and γ subgroups of the Proteobacteria (Hanson et al., Microb. Growth C1 Compounds., [Int. Symp.], 7th (1993), 285-302. Editor(s): Murrell, J. Collin; Kelly, Don P. Publisher: Intercept, Andover, UK; Madigan et al., Brock Biology of Microorganisms, 8th edition, Prentice Hall, UpperSaddle River, N.J. (1997)). Facultative methylotrophic bacteria suitable in the present invention include, but are not limited to: Methylophilus, Methylobacillus, Methylobacterium, Hyphomicrobium, Xanthobacter, Bacillus, Paracoccus, Nocardia, Arthrobacter, Rhodopseudomonas, and Pseudomonas.

Those methylotrophs having the additional ability to utilize methane as a primary carbon source are referred to as methanotrophs. Of particular interest in the present invention are those obligate methanotrophs which are methane utilizers but which are obliged to use organic compounds lacking carbon-carbon bonds. Exemplary organisms included in this classification of obligate methanotrophs that utilize C₁ compounds are the genera Methylomonas, Methylobacter, Methylococcus, Methylosinus, Methylocyctis, Methylomicrobium, and Methanomonas, although this is not intended to be limiting.

Of particular interest in the present invention are high growth obligate methanotrophs having an energetically favorable carbon flux pathway. For example, a specific strain of methanotroph having several pathway features that makes it particularly useful for carbon flux manipulation is known as Methylomonas 16a (ATCC PTA 2402) (U.S. Pat. No. 6,689,601). This particular strain and other related methylotrophs including for example, Methylomonas clara and Methylosinus sporium, are preferred microbial hosts for expression of numerous gene products. These strains have both the expected Entner-Douderoff Pathway (which utilizes the keto-deoxy phosphogluconate aldolase enzyme) and in addition, the Embden-Meyerhof Pathway (which utilizes the fructose bisphosphate aldolase enzyme). Energetically, the latter pathway is most favorable and allows greater yield of biologically useful energy, ultimately resulting in greater yield production of cell mass and other cell mass-dependent products.

Methylomonas sp. 16a (ATCC PTA-24202) is normally pink in color due to production of C₃₀ carotenoids. For visual screening of C₄₀ carotenoid production, C₃₀ carotenoid production was eliminated in the strain to provide a non-pigmented background. The process used to create the non-pigmented strain used in the present examples (e.g., Methylomonas sp. MWM1200 (ATCC PTA-6887)) is described in copending U.S. patent application Ser. No. 10/997,844; hereby incorporated by reference. Briefly, several genes involved in the production of C₃₀ carotenoids (i.e. crtN1, ald, crtN2, and crtN3) were disrupted, resulting an a non-pigmented strain of Methylomonas optimized for engineering C₄₀ carotenoid production.

In one embodiment, suitable host cells are methylotrophic bacteria. In another embodiment, the methylotroph is a methanotroph. In yet another embodiment, the methanotroph is a high growth methanotroph. In a further embodiment, the high growth methanotroph is Methylomonas sp. 16a (ATCC 2402) and derivatives thereof.

In one embodiment, the C₁ carbon source is any organic molecule lacking a carbon to carbon bond. In another embodiment, the C₁ carbon source is methanol and/or methane. In yet another embodiment, the host cell is a methylotroph grown using methanol and/or methane as a carbon source. In yet a further embodiment, the methylotrophic host cell is a methanotroph grown using methanol and/or methane as a carbon source.

Integration Stability

For commercial production economics, it is desirable to use a genetically stable microbial host. Stability of the introduced genes should be maintained over multiple generations. Chromosomal integration in the fliC region provides this level of stability. Chromosomal insertion provides the most segregationally stable expression system for foreign DNA since the foreign DNA is passed on to progeny as a part of normal chromosomal replication and since, theoretically, the foreign DNA can only be lost as a result of a recombination event.

As used herein, the term “stably expressed” or “stable expression” refers to an integration event that results in the expression of the integrated nucleic acid molecule for at least about 10 generations in the transformed host cells. In one aspect, stability is measured over at least 10 generations and is observed in at least about 90% of the transformed host cells comprising a chromosomal integration in the fliC region. In another aspect, stability is observed in at least about 98% of the transformed host cells comprising a chromosomal integration in the fliC region.

In Vivo Transposition for the Integration of Promoterless Reporter Transposons

The in vivo transposition vector pUTminiTn5gfpTet (GenBank® AY364166) provided plasmid and transposon functions used to construct a promoterless transposon vector (Matthysse et al., FEMS Microbiol. Lett. 145:87-94 (1996); de Lorenzo et al., J. Bacteriol., 172(11):6568-6572 (1990); Herrero et al., J. Bacteriol. 172(11):6557-6567 (1990)). The pUTminiTn5gfpTet plasmid is comprised of the IS50r transposase gene (a modified wild type tnp tranposase with the NotI site removed; Auerswald et al., Cold Spring Harb. Symp. Quant. Biol. 4 (part 1): 107-113 (1981); Ahmed et al., Gene 154(1):129-130 (1995)), an R6K origin of replication, an OriT(RP4) origin of transfer (GenBank® X54459), a gfp gene encoding a mutant green fluorescent protein, a bla gene encoding a beta-lactamase, and a tetA gene encoding a class C tetracycline resistance protein.

The parent plasmid, pUT, is a derivative of the pGP704 plasmid (de Lorenzo et al., supra; Miller and Mekalanos, J. Bacteriol., (170): 2575-2583 (1988) and was used to create pUTminiTn5gfpTet. Plasmid pGP704 is a derivative of pBR322 that is Amp^{R} but has a deletion of the pBR322 origin of replication (oriE1). Instead, the plasmid contains a cloned fragment containing the origin of replication of plasmid R6K. The R6K origin of replication (oriR6K) requires the Π protein, encoded by the pir gene. In E. coli, the Π protein can be supplied in trans by a prophage (λ pir) that carries a cloned copy of the pir gene. The pGP704 plasmid also contains a 1.9 kB BamHI fragment encoding the mob region of RP4. Thus, pGP704 (and the present pUT derivatives thereof) can be mobilized into recipient strains by transfer functions provided by a derivative of RP4 integrated in the chromosome of E. coli strain SM 10 or SY327. Once the plasmid is transferred, however, it is unable to replicate in recipients that lack the Π protein (e.g., recipients such as Methylomonas and other methylotrophic bacteria). Use of the pGP704 plasmid, and derivatives thereof, for genetically engineering Methylomonas sp. has been previously described in U.S. Ser. No. 10/997,308 and U.S. Ser. No. 10/997,844; hereby incorporated by reference.

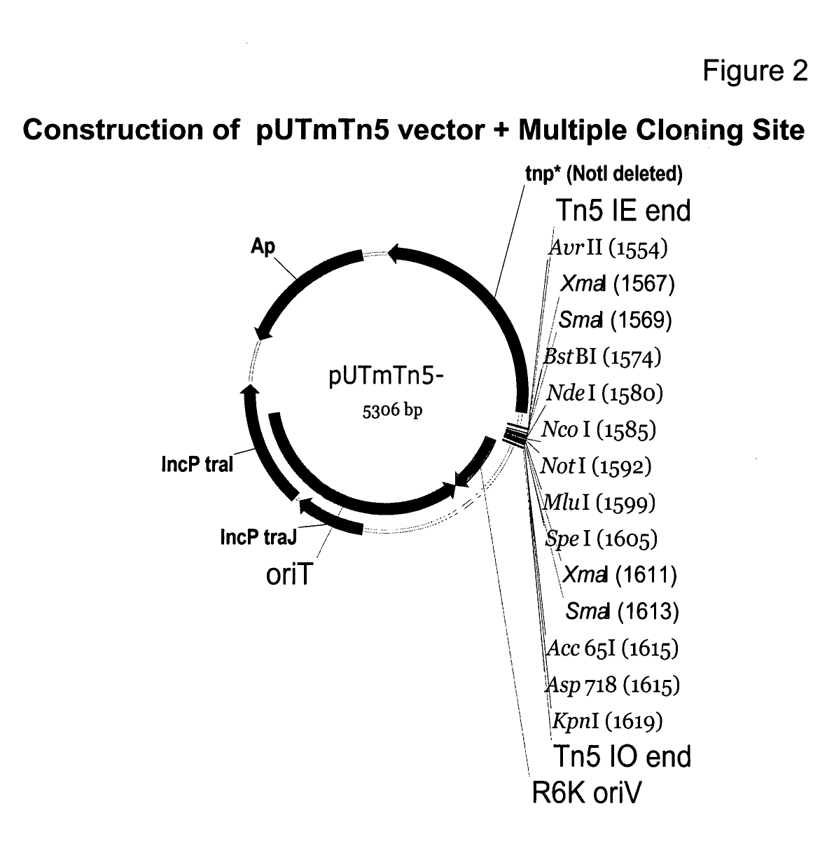

A modified version of the pUTminiTn5gfpTet plasmid was created by removing the gfp and tet genes, leaving intact the plasmid functions, the gene encoding the tranposase, and the ends of the Tn5 transposon (inverted repeats, typically about 19 base pairs in length, referred to at “IE” and “IO” ends in FIG. 2). A multiple cloning site (MCS) was subsequently added, creating plasmid pUTmTn5. Various promoterless constructs (carotenoid biosynthesis gene clusters) were cloned into the MCS to create the promoterless astaxanthin transposons used to identify suitable chromosomal integration sites.

The mobilization of the pUTmTn5 plasmids into Methylomonas occurs through conjugation. Once in the host cell, the tranposase inserts the astaxanthin transposon (or canthaxanthin transposon) randomly throughout the entire genome. Insertion of the promoterless carotenoid producing transposon (canthaxanthin or astaxanthin) in regions that are actively transcribed are easily identified by the generation of pigment as an endogenous chromosomal promoter drives expression of the promoterless DNA insert encoding several carotenoid biosynthesis enzymes (the non-pigmented strain Methylomonas sp. MWM1200 was used as the background). Survival and growth of the pigmented cells indicated that the insertion regions did not encode genes essential for survival (assuming a single copy of each). Stability of the chromosomal insertion sites was determined by growing the pigmented cells for several generations, measuring the frequency of those cells that loose the ability to produce the reporter molecule. In one embodiment, stable chromosomal integration sites are those that are able to maintain the transposon (as visually indicated by the presence of pigmentation) in the vast majority (i.e. less than about 1%) of the transformed host cells over at least about 10 generations. In another embodiment, insertion sites are considered stable if the vast majority of the cells retain their pigmentation over at least about 15 generations. In yet another embodiment, insertion sites are considered stable if the vast majority of the cells retain their pigmentation over at least about 50 generations.

Use of the mini-Tn5 transposase system is exemplified. However, the use of other transposable elements in combination with a transposase for both in vivo and in vitro transposition are known in the art. Kits for in vitro transposition are commercially available (see for example The Primer Island Transposition Kit, available from Perkin Elmer Applied Biosystems, Branchburg, N.J., based upon the yeast Ty1 element; The Genome Priming System, available from New England Biolabs, Beverly, Mass.; based upon the bacterial transposon Tn7; and the EZ::TN Transposon Insertion Systems, available from Epicentre Technologies, Madison, Wis., based upon the Tn5 bacterial transposable element.

Composition of the FliC Region

High-level astaxanthin or canthaxanthin production was observed when the respective promoterless gene cluster was integrated into the fliC region of a methylotrophic bacterial cell (Methylomonas sp. 16a). An ORF was identified that encoded a protein with high amino acid similarity to the flagellin subunit protein. The flagellin subunit gene encodes a protein that is commonly found across a wide range of bacterial species, sharing many conserved amino acid residues and making it possible to identify flagellin genes by sequence similarity (Millikan et al., Appl. Environ. Microbiol., 65(7):3129-3133 (1999)). Thus, derivatives of Methylomonas sp. 16a with interruption of the flagellin subunit protein expression were viable and showed high expression based on the presence of pigment.

Further sequence analysis of the region surrounding the fliC gene showed that this fliC gene is one ORF in an operon that includes four ORFs with the same orientation that all encode proteins involved in flagellar motility (FIG. 5). The first open reading frame of this cluster (fliC; SEQ ID NO: 35) encodes a protein (SEQ ID NO: 36) with sequence similarity to the flagellin subunit protein, FliC. The second ORF in the cluster (flaG; SEQ ID NO: 37) encodes a protein (SEQ ID NO: 38) with similarity to FlaG (protein involved in forming the export apparatus for flagellar proteins), the third ORF (fliD; SEQ ID NO: 39) encodes a protein (SEQ ID NO: 40) with sequence similarity to FliD (flagellar hook-associated capping protein), and the fourth ORF (fliS; SEQ ID NO: 41) encodes a protein (SEQ ID NO: 42) with similarity to FliS (flagellin specific chaperone). This cluster of genes is structured such that each gene does not have its own promoter, but a promoter for expression of the entire cluster lies upstream of the fliC gene. This fliC promoter (SEQ ID NO: 34) directs the transcription of the entire fliC-flaG-fliD-fliS genomic region. The sequence of the entire fliC region identified in Methylomonas sp. 16a is represented by SEQ ID NO: 33.

Several transformed strains were identified with insertions located in different open reading frames within the fliC region (FIG. 5). A gene integrated within this fliC region and operably linked to the fliC promoter will be transcribed along with the other genes in the cluster. Thus, for expression, an integrated gene must be 3′ to the promoter for the fliC region. All of the coding regions in the fliC region gene cluster are oriented with the same 5′ to 3′ polarity. An introduced gene must be integrated such that the orientation of the coding region is the same as the orientation of the other coding regions in the fliC region gene cluster.

A gene may be integrated in the fliC region in any location that facilitates expression and does not compromise the host strain. It is obvious to one skilled in the art that integration within a coding region of the fliC region gene cluster would affect expression of the encoded protein. However, integration of foreign DNA within an ORF in the fliC region does not adversely affect the viability and growth rate of the transformed host cell. In another aspect, it may be desirable to integrate a gene into an intergenic region within the fliC region to avoid disruption of the expression of any encoded proteins and to ensure function of the expressed introduced gene product. Knowledge of the integration region sequence allows one of skill to target the integration of a foreign DNA fragment using methods well-known in the art (see for example, use of an integration vector and homologous recombination as described in U.S. Ser. No. 10/997,308 and U.S. Ser. No. 10/997,844; hereby incorporated by reference).

Strategy for Identification of High Expression Integration Regions

Transposons comprised of a promoterless carotenoid gene cluster were randomly introduced at a number of sites in the host genome and screened for the production of a carotenoid pigment (e.g. canthaxanthin or astaxanthin). It will be appreciated that the same process could be accomplished using more standard markers such as β-galactosidase, β-glucuronidase, or other genes that express an enzyme that can metabolize a colorless substrate. In the context of the present invention, the carotenoid produced was astaxanthin or canthaxanthin; providing a strong visual marker indicative of expression. In addition, the size of the insert was more than 5 kB, indicating that the insertion site can support a stable expression of a relatively large gene cluster.

In another aspect of the invention, the integration site identified using the present method can be used to incorporate one or more genes lacking a promoter. In this way, the endogenous promoter controlling expression of the identified region is used to drive expression of the foreign DNA inserted. In yet a further embodiment, the endogenous fliC promoter (SEQ ID NO: 34) can be isolated and used to drive chimeric gene expression at additional integration sites within the host genome.

In another embodiment, DNA constructs comprised of at least one promoter operably linked to one or more coding sequences can be inserted into the identified integration regions. In this way, insertion of a construct comprised of a foreign promoter takes advantage of the stable, non-essential nature of the integration region (i.e. disruption of the expression of the endogenous genes within the region is not significantly detrimental to the survival and/or growth rate of the host cell).

The genomic DNA from the pigmented transformed cells can then be characterized to identify the integration site of the reporter gene(s) through sequencing the DNA surrounding the integrated reporter gene(s). Primers can be designed based on the sequence of the promoteriess transposon constructs so that the chromosomal regions flanking the insertion site can be sequenced. Further analysis of the surrounding DNA sequences using sequence analysis software such as the GCG suite of programs ((Wisconsin Package Version 9.0, Genetics Computer Group (GCG), Madison, Wis.); BLASTP, BLASTN, BLASTX (Altschul et al., J. Mol. Biol., 215:403-410 (1990)); DNASTAR (DNASTAR, Inc., Madison, Wis.); and the FASTA program incorporating the Smith-Waterman algorithm (W. R. Pearson, Comput. Methods Genome Res., [Proc. Int. Symp.], Meeting Date 1992, 111-20. Suhai, Sandor, Ed.; Plenum: New York, N.Y. (1994)) locates ORFs (including orientation) and determines the identities of those ORFs through DNA or protein homology to known sequences. A map of ORFs and putative promoter regions may be constructed based on the results of the sequence analysis. The map allows the determination of how the integrated gene is being expressed: what promoter is used, and whether it is part of an operon.

Suitable Integration Sites within the FliC Region

Foreign DNA (e.g. genes) can be stably inserted and expressed anywhere with the fliC region including open reading frames and the corresponding intergenic regions flanking the ORFS. In one aspect, the integration site can be anywhere within the region operably linked and expressed under the control of the endogenous fliC promoter. In another aspect, a suitable integration site within the fliC region of a methylotrophic microorganism has at least 95% identity to a nucleic acid sequence selected from the group consisting of SEQ ID NOs: 33, 35, 37, 39, and 41. In yet another aspect, the integration site has at least 95% identity to a nucleic acid sequence encoding an amino acid sequence selected from the group consisting of SEQ ID NOs: 36, 38, 40, and 42. In a further aspect, the integration site within the fliC region is a nucleic acid sequence encoding an amino acid sequence selected from the group consisting of SEQ ID NOs: 36 and 42. In yet a further aspect, the integration site within the fliC region comprises a nucleic acid sequence selected from the group consisting of SEQ ID NOs: 33, 35, 37, 39, and 41.

The fliC region within a methylotroph comprises at least one open reading frame encoding a flagellin subunit protein. In another aspect, the fliC region is comprised of at least 4 ORFS having the following organization: fliC-flaG-fliD-fliS. In yet another aspect, the fliC region refers to the region of chromosomal DNA comprising of one or more open reading frames that are expressed from a nucleic acid molecule encoding the fliC promoter having at least 95% identity to the SEQ ID NO: 34. In yet another aspect, the fliC promoter is represented by SEQ ID NO: 34.

Targeted Integration of Suitable Integration Sites

Once the location and sequence of a suitable integration region is identified by the screening methods described herein, an integration vector may be used for targeted integration of a gene(s) into the targeted region, providing that the vector contains a DNA sequence that is homologous to a portion of the genomic target region. Regions of homology are designed using the sequence of the desired insertion site and may be as short as about 0.5 kB in length, is preferably of at least about 1 kB in length and more preferred is at least about 1 to 2.4 kB in length.

Homologs of the Methylomonas sp. 16a FliC Region

One or more of the present sequences can be used to identify substantially similar fliC regions in other methylotrophic microorganisms. The skilled artisan recognizes that substantially similar nucleotide sequences encompassed by this invention are also defined by their ability to hybridize, particularly under highly stringent conditions, with the sequences exemplified herein.

Typically, stringent conditions are those in which the salt concentration is less than about 1.5 M Na ion (typically about 0.01 to 1.0 M Na ion concentration or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved by adding destabilizing agents such as formamide. Exemplary stringency conditions include hybridization with a buffer solution of 6×SSC (1 M NaCl), 30 to 35% formamide, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 6×SSC (1 M NaCl), 40 to 45% formamide, 1% SDS at 37° C., and a wash in 0.5× to 1×SSC at 55 to 60° C. Exemplary high stringency conditions include hybridization in 0.1×SSC, 0.1% SDS, at 65° C. and a wash with 2×SSC, 0.1% SDS followed by 0.1×SSC, 0.1% SDS at a temperature of 65° C.). Hybridization and washing conditions are well known and exemplified in Sambrook, et al., supra; particularly Chapter 11 and Table 11.1.).

A fliC region (or any ORF within the fliC region) may also be identified through sequence analysis of genomic DNA sequences using sequence analysis software, or may be cloned using a probe made from the Methylomonas sp. 16a fliC region, preferably from the fliC coding sequence. In one embodiment, substantially similar nucleic acid fragments of the instant invention are those nucleic acid fragments whose DNA sequences are at least about 80% identical to the DNA sequence of the nucleic acid fragments reported herein. In another embodiment, substantially similar nucleic acid fragments are at least about 90% identical to the DNA sequence of the nucleic acid fragments reported herein. In yet a further embodiment, substantially similar nucleic acid fragments are at least about 95% identical to the DNA sequence of the nucleic acid fragments reported herein. In still a further embodiment, substantially similar nucleic acid fragments are at least about 98% identical to the DNA sequence of the nucleic acid fragments reported herein.

Genes for Integration in the FliC Region

Metabolic engineering generally requires the introduction of one or more genes whose expression leads to altered metabolism. It is usually desired that the introduced genes exhibit high level expression. In cases where a product is to be produced through large scale growth in a bioreactor, the lack of a selection marker, stability of the introduced gene, and normal growth rate of the host microorganism are also important. Thus for many metabolic engineering projects, integration in the fliC region may provide the desired properties. Any gene that is useful for metabolic engineering may be integrated in the fliC region. Additionally, genes encoding proteins that in themselves are of commercial value may be expressed in the fliC region integration system. The genes for integration may be either endogenous to the host or heterologous and must be compatible with the host organism. For example, “suitable genes of interest” may include, but are not limited to those encoding viral, bacterial, fungal, plant, insect, or vertebrate proteins of interest, including mammalian polypeptides. Further, these “genes of interest” may be, for example, structural proteins, enzymes, or peptides. As will be obvious to one skilled in the art, the particular functionalities required to be introduced into a host organism for production of a particular product will depend on the host cell, the availability of substrate, and the desired end product(s). In one aspect, a “coding region of interest” is defined herein as a nucleic acid molecule that includes, but is not limited to those encoding viral, bacterial, fungal, plant, insect, or vertebrate proteins of interest, including mammalian polypeptides. In another aspect, the “coding region of interest” encodes enzymes involved in isoprenoid biosynthesis, carotenoid biosynthesis, central carbon metabolism, exopolysaccharide production, and aromatic acid production. In a further aspect, the coding region of interest is a cluster of one or more coding regions that can be expressed together when operably linked to a suitable promoter. In a preferred aspect, the coding region of interest is one that, when operably linked to a suitable promoter, can be functionally expressed as a chimeric gene in a transformed host cell.

A particularly preferred, but non-limiting list of genes includes:
1) genes encoding enzymes involved in the central carbon pathway, such as transaldolase, fructose bisphosphate aldolase, keto deoxy phosphogluconate aldolase, phosphoglucomutase, glucose-6-phosphate isomerase, phosphofructokinase, 6-phosphogluconate dehydratase, and 6-phosphogluconate-6-phosphate-1 dehydrogenase;
2) genes encoding enzymes involved in the production of isoprenoid molecules, such as 1-deoxyxylulose-5-phosphate synthase (dxs), 1-deoxyxylulose-5-phosphate reductoisomerase (dxr), geranyltransferase or farnesyl diphosphate synthase (ispA), 2C-methyl-D-erythritol cytidyltransferase (ispD), to 4-diphosphocytidyl-2-C-methylerythritol kinase (ispE), 2C-methyl-d-erythritol 2,4-cyclodiphosphate synthase (ispF), 2-C-methyl-D-erythritol 4-phosphate synthase (ispG); CTP synthase (pyrG)), and isopentenyl diphosphate isomerase (idi);
3) genes encoding carotenoid pathway enzymes such as geranylgeranyl pyrophosphate synthase (crtE); zeaxanthin glucosyl transferase (crtX), lycopene cyclase (crtY), phytoene desaturase (crtI), phytoene synthase (crtB), carotenoid hydroxylase (crtZ), and carotenoid ketolase (crtO, crtW and bkt);
4) genes encoding enzymes involved in the production of exopolysaccharides, such as UDP-glucose pyrophosphorylase (ugp), glycosyltransferase (gumD), polysaccharide export proteins (wza, espB), polysaccharide biosynthesis (espM), glycosyltransferase (waaE), sugar transferase (espV), galactosyltransferase (gumH), and glycosyltransferase genes;
5) genes encoding enzymes involved in the production of aromatic amino acids, such as 3-deoxy-D-arabinoheptulosonate-7-phosphate synthase (aroG), 3-dehydroquinate synthase (aroB), 3-dehydroquinase or 3 dehydroquinate dehydratase (aroQ), 5-shikimic acid dehydrogenase (aroE), shikimic acid kinase (aroK), 5-enolpyruvylshikimate-3-phosphate synthase, chorismate synthase (aroC), anthranilate synthase (trpE), anthranilate phosphoribosyltransferase (trpD), indole 3-glycerol phosphate synthase (trpC), tryptophan synthetase (trpB), chorismate mutase or prephenate dehydratase (pheA), and prephenate dehydrogenase (tyrAc); and
6) pds, phaC, phaE, efe, pdc, and adh genes and genes encoding pinene synthase, bornyl synthase, phellandrene synthase, cineole synthase, sabinene synthase, and taxadiene synthase, respectively.

The preferred genes of 3) above include, but are not limited to crtE, crtB, crtI, crtY, crtZ, crtW and crtX genes isolated from Pectobacterium cypripedii DC416, as described in U.S. Ser. No. 10/804,677; crtE, crtB, crtI, crtY, crtZ and crtX genes isolated from a member of the Enterobacteriaceae DC260 family, as described in U.S. Ser. No. 10/808,979; crtE, idi, crtB, crtI, crtY, crtZ genes isolated from Pantoea agglomerans DC404, as described in U.S. Ser. No. 10/808,807; crtE, idi, crtB, crtI, crtY, crtZ and crtX genes isolated from Pantoea stewartii DC413, as described in U.S. Ser. No. 10/810,733; the crtW and crtZ genes from Agrobacterium aurantiacum, as described in U.S. Ser. No. 10/997,844, the crtW and crtZ genes from Brevundimonas vesicularis DC263 as described in U.S. Ser. No. 11/015,433, and the crtW gene from Sphingomonas melonis DC18 or Flavobacterium sp. K1-202C, as described in U.S. Ser. No. 11/015,433.

For coding regions with codon usage that is not optimal for expression in the host bacterium, it is desirable to modify a portion of the codons to enhance the expression the encoded polypeptides in a methylotroph, or specifically in Methylomonas sp. 16a and derivatives thereof. For example, the nucleic acid sequence of the native β-carotene ketolase gene (crtW) from Agrobacterium aurantiacum was modified to employ host preferred codons for expression in Methylomonas sp. 16a (U.S. Ser. No. 10/997,844). In general, host preferred codons can be determined from the codons of highest frequency in the proteins (preferably expressed in the largest amount) in a particular host species of interest. Thus, the coding sequence for a polypeptide having ketolase activity can be synthesized in whole or in part using the codons preferred in the host species. All (or portions) of the DNA also can be synthesized to remove any destabilizing sequences or regions of secondary structure which would be present in the transcribed mRNA. All (or portions) of the DNA also can be synthesized to alter the base composition to one more preferable in the desired host cell.

As is well known to those of skill in the art, efforts to genetically engineer a microorganism for high-level production of a specific product frequently require high-level expression of one or more introduced genes. For large-scale production, the introduced gene(s) must be stably maintained, preferably without the requirement for an antibiotic or nutritional selection.

In one aspect, the fliC region is used for expression of genes encoding enzymes involved in carotenoid synthesis in an any methylotrophic microorganism. In another aspect, the methylotrophic microorganism is a methylotrophic bacteria, providing a new platform for production of carotenoids. In another aspect, the fliC region is used for expression of genes for C₄₀ carotenoid synthesis in Methylomonas sp. 16a (and in derivatives thereof) providing a platform for production of C₄₀ carotenoids including, but are not limited to antheraxanthin, adonirubin, adonixanthin, astaxanthin, canthaxanthin, capsorubrin, β-cryptoxanthin, α-carotene, β-carotene, epsilon-carotene, echinenone, 3-hydroxyechinenone, 3′-hydroxyechinenone, γ-carotene, 4-keto-γ-carotene, ζ-carotene, α-cryptoxanthin, deoxyflexixanthin, diatoxanthin, 7,8-didehydroastaxanthin, fucoxanthin, fucoxanthinol, isorenieratene, lactucaxanthin, lutein, lycopene, myxobactone, neoxanthin, neurosporene, hydroxyneurosporene, peridinin, phytoene, rhodopin, rhodopin glucoside, 4-keto-rubixanthin, siphonaxanthin, spheroidene, spheroidenone, spirilloxanthin, 4-keto-torulene, 3-hydroxy-4-keto-torulene, uriolide, uriolide acetate, violaxanthin, zeaxanthin-β-diglucoside, and zeaxanthin. Preferred carotenoids produced by the present methods include β-carotene, lycopene, zeaxanthin, canthaxanthin, and astaxanthin. In a further preferred aspect, the carotenoids are canthaxanthin and/or astaxanthin.

Carotenoid Biosynthesis Genes

There is a general practical utility for microbial production of C₄₀ carotenoid compounds. These compounds are very difficult to make chemically (Nelis and Leenheer, Appl. Bactedol. 70:181-191 (1991)). Industrially, only a few carotenoids are used for food colors, animal feeds, pharmaceuticals, and cosmetics, despite the existence of more than 600 different carotenoids identified in nature. Most carotenoids have strong color and can be viewed as natural pigments or colorants. Furthermore, many carotenoids have potent antioxidant properties and thus inclusion of these compounds in the diet is thought to provide health benefits. Carotenoids produced in a microbial host may be used as a part of the single cell protein product, or may be purified prior to use.

The synthesis of carotenoids occurs through the upper carotenoid pathway providing for the conversion of pyruvate and glyceraldehyde-3-phosphate to farnesyl pyrophosphate (FPP) and the lower carotenoid biosynthetic pathway that provides for the synthesis of either diapophytoene (C₃₀) or phytoene (C₄₀) and all subsequently produced carotenoids. The genetics of carotenoid biosynthesis are well-known (Armstrong, G., in Comprehensive Natural Products Chemistry, Elsevier Press, volume 2, pp 321-352 (1999)); Lee, P. and Schmidt-Dannert, C., Appl Microbiol Biotechnol, 60:1-11 (2002); Lee et al., Chem Biol 10:453-462 (2003), and Fraser, P. and Bramley, P. (Progress in Lipid Research, 43:228-265 (2004)). This pathway is extremely well studied in the Gram-negative, pigmented bacteria of the genera Pantoea, formerly known as Erwinia. Of particular interest are the genes responsible for the production of C₄₀ carotenoids used as pigments in animal feed (e.g. canthaxanthin and astaxanthin).

For the biosynthesis of C₄₀ carotenoids, a series of enzymatic reactions catalyzed by CrtE and CrtB occur to convert FPP to geranylgeranyl pyrophosphate (GGPP) to phytoene, the first 40-carbon molecule of the lower carotenoid biosynthesis pathway. From the compound phytoene, a spectrum of C₄₀ carotenoids are produced by subsequent hydrogenation, dehydrogenation, cyclization, oxidation, or any combination of these processes. Lycopene, which imparts a “red”-colored spectra, is produced from phytoene through four sequential dehydrogenation reactions by the removal of eight atoms of hydrogen, catalyzed by phytoene desaturase (encoded by the gene crtI). Lycopene cyclase (encoded by the gene crtY) converts lycopene to β-carotene. β-carotene can be converted to astaxanthin by the combination of at least one β-carotene ketolase (encoded by a crtWIbkt or crtO gene) and at least one carotenoid hydroxylase (encoded by a crtZ or crtR gene). Thus, the set of genes crtE, crtB, crt I, crtY, crtW, and crtZ together encode a biosynthetic pathway for the conversion of FPP to astaxanthin. These genes can be linked together with all coding regions in the same orientation such that expression of one DNA fragment provides for the synthesis of astaxanthin from FPP.

Industrial Production Methodologies

Where expression of one or more genes of interest is desired using the fliC region, a variety of culture methodologies may be applied. For example, large-scale production of a specific product made possible by integrated gene expression in a recombinant microbial host may be accomplished by both batch and continuous culture methodologies.

A classical batch culturing method is a closed system where the composition of the media is set at the beginning of the culture and not subject to external alterations during the culturing process. Thus, at the beginning of the culturing process the media is inoculated with the desired organism or organisms and growth or metabolic activity is permitted to occur while adding nothing to the system. Typically, however, a “batch” culture is batch with respect to the addition of carbon source and attempts are often made at controlling factors such as pH and oxygen concentration. In batch systems the metabolite and biomass compositions of the system change constantly up to the time the culture is terminated. Within batch cultures cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the Fed-Batch system. Fed-Batch culture processes are also suitable in the present invention and comprise a typical batch system with the exception that the substrate is added in increments as the culture progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch culturing methods are common and well known in the art and examples may be found in Thomas D. Brock in Biotechnology: A Textbook of Industrial Microbiology, 2^{nd} ed. (1989) Sinauer Associates: Sunderland, Mass., or Deshpande, Mukund V., Appl. Biochem. Biotechnol., 36:227 (1992).

Commercial production of a product of interest in a methylotrophic bacteria may also be accomplished with a continuous culture. Continuous cultures are an open system where a defined culture media is added continuously to a bioreactor and an equal amount of conditioned media is removed simultaneously for processing. Continuous cultures generally maintain the cells at a constant high liquid phase density where cells are primarily in log phase growth. Alternatively continuous culture may be practiced with immobilized cells where carbon and nutrients are continuously added, and valuable products, by-products and waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the culture. Methods of modulating nutrients and growth factors for continuous culture processes, as well as techniques for maximizing the rate of product formation, are well known in the art of industrial microbiology and a variety of methods are detailed by Brock, supra.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### General Methods

Standard recombinant DNA and molecular cloning techniques used in the Examples are well known in the art and are described by Sambrook, J., Fritsch, E. F. and Maniatis, T. Molecular Cloning: A Laboratory Manual; Cold Spring Harbor Laboratory: Cold Spring Harbor, N.Y. (1989) (“Maniatis”); by T. J. Silhavy, M. L. Bennan, and L. W. Enquist, Experiments with Gene Fusions, Cold Spring Harbor Laboratory: Cold Spring Harbor, N.Y. (1984); and by Ausubel, F. M. et al., Current Protocols in Molecular Biology, published by Greene Publishing Assoc. and Wiley-Interscience, Hoboken, N.J. (1987). Polymerase Chain Reactions (PCR) techniques can be found in White, B., PCR Protocols: Current Methods and Applications, Humana: Totowa, N.J. (1993), Vol. 15.

General materials and methods suitable for the maintenance and growth of bacterial cultures are found in: Experiments in Molecular Genetics (Jeffrey H. Miller), Cold Spring Harbor Laboratory: Cold Spring Harbor, N.Y. (1972); Manual of Methods for General Bacteriology (Phillip Gerhardt, R. G. E. Murray, Ralph N. Costilow, Eugene W. Nester, Willis A. Wood, Noel R. Krieg and G. Briggs Phillips, eds.), American Society for Microbiology: Washington, D.C., pp 210-213; or, Thomas D. Brock in Biotechnology: A Textbook of Industrial Microbiology, 2^{nd} ed. Sinauer Associates: Sunderland, Mass. (1989).

The meaning of abbreviations is as follows: “sec” means second(s), “min” means minute(s), “hr” means hour(s), “d” means day(s), “μL” means microliter(s), “mL” means milliliter(s), “L” means liter(s), “μM” means micromolar, “mM” means millimolar, “M” means molar, “mmol” means millimole(s), “μmol” mean micromole(s), “nmol” means nanomole(s), “pmol” means picomole(s), “g” means gram(s), “μg” means microgram(s), “ng” means nanogram(s), “nm” means nanometers, “U” means unit(s), “ppm” means parts per million, “bp” means base pair(s), “rpm” means revolutions per minute, “kB” means kilobase(s), “g” means the gravitation constant, “MW” means molecular weight, “Conc.” means concentration, “Kn” or “Kn^{r}” means kanamycin resistance gene, “Cm” or “CM^{r}” means chloramphenicol resistance gene, “OD₆₀₀” means the optical density measured at 600 nm, “OD₂₆₀/OD₂₈₀” means the ratio of the optical density measured at 260 nm to the optical density measured at 280 nm, and “mAU” means milliabsorbance units.

All reagents and materials used for the growth and maintenance of bacterial cells were obtained from Aldrich Chemicals (Milwaukee, Wis.), BD Diagnostic Systems (Sparks, Md.), Invitrogen Corp. (Carlsbad, Calif.), or Sigma Chemical Company (St. Louis, Mo.), unless otherwise specified.

### Example 1

### Construction of Promoterless Carotenoid Transposons

Promoterless carotenoid transposons were constructed for the purpose of identifying chromosomal insertions site that support high-level carotenoid gene expression and stable carotenoid production.

The in vivo transposition vector pUTminiTn5gfpTet provided essential plasmid and transposon functions used to construct a promoterless carotenoid transposon vector. The carotenoid genes necessary for canthaxanthin or astaxanthin production were taken from carotenoid plasmids pDCQ334 (SEQ ID NO: 1), pDCQ341 (SEQ ID NO: 2), pDCQ343 (SEQ ID NO: 3), or pDCQ377 (SEQ ID NO: 4). In addition, the kanamycin resistance gene (Kn^{R}) was PCR amplified from EZ::TN™<Kan-2> (Epicentre, Madison, Wis.).

### Preparation of Several Carotenoid Gene Cluster Expression Plasmids

Plasmid pDCQ334 (Astaxanthin Gene Cluster)

Plasmid pDCQ334 (SEQ ID NO: 1) was created by cloning into the broad host range plasmid pBHR1 (MoBiTec GmbH, Goettingen, Germany) codon-optimized versions of the crtW ketolase gene and crtZ hydroxylase gene from Agrobacterium aurantiacum (U.S. Ser. No. 10/997,844, hereby incorporated by reference) immediately upstream of the crtEidiYIB gene cluster from Pantoea agglomerans DC404 (U.S. Ser. No. 10/808,807; hereby incorporated by reference) forming the gene cluster crtWZEidiYIB (SEQ ID NO: 5) operably linked to the chloramphenicol resistance gene promoter (P_{cat}) on pBHR1. Transposon vector pUTmTn5-334 was prepared by cloning the promoterless crtWZEidiYIB gene cluster from pDCQ334 into pUTmTn5.

Plasmid pDCQ341 (Canthaxanthin Gene Cluster)

Plasmid pDCQ341 (SEQ ID NO: 2) was created by cloning into plasmid pBHR1 the Sphingomonas melonis DC18 crtW ketolase gene (SEQ ID NO: 15; U.S. Ser. No. 11/015,433; hereby incorporated by reference) immediately upstream of the crtEYIB gene cluster from Enterobacteriaceae DC260 (U.S. Ser. No. 10/808,979; hereby incorporated by reference) forming a crtWEYIB carotenoid gene cluster (SEQ ID NO: 6) operably linked to the P_{cat} promoter. Transposon vector pUTmTn5-341 Kn was prepared by eliminating the crtZ coding region from transposon cloning vector pUTmTn5-343Kn.

Plasmid pDCQ343 (Astaxanthin Gene Cluster)

Plasmid pDCQ343 (SEQ ID NO: 3) was created by cloning into plasmid pDCQ341 the Brevundimonas vesicularis DC263 crtZ hydroxylase (SEQ ID NO: 16; U.S. 60/601,947) into the crtWEYIB gene cluster forming a crtWZEYIB carotenoid gene cluster (SEQ ID NO: 7) operably linked to the P_{cat} promoter. Transposon vector pUTmTn5-343 was prepared by cloning the promoterless crtEYIB cluster from plasmid pDCQ343 to create pUTmTn5-343EYIB. The promoterless crtWZ gene cluster was PCR amplified using the pDCQ343 plasmid as a template. The amplified fragment was subsequently cloned upstream of the crtEYIB cluster in pUTmTn5-343EYIB, creating transposon vector pUTmTn5-343.

Plasmid pDCQ377 (Astaxanthin Gene Cluster)

Plasmid pDCQ377 (SEQ ID NO: 4) was created by cloning into plasmid pBHR1 the crtW gene and the crtZ gene from Brevundimonas vesicularis DC263 (U.S. Ser. No. 11/015,433 and U.S. 60/601,947) immediately upstream of the crtEidiYIB gene cluster from Pantoea agglomerans DC404 (U.S. Ser. No. 10/808,807; hereby incorporated by reference) forming a crtWZEidiYIB carotenoid gene cluster (SEQ ID NO: 8) operably linked to the P_{cat} promoter. Transposon vector pUTmTn5-377Kn was created by removing the carotenoid gene cluster from pUTmTn5-334Kn and inserting the promoterless crtWZEidiYIB cluster from plasmid pDCQ377.

### Preparation of the pUTmTn5gfpTet Vector DNA

The pUTmTn5gfpTet vector DNA (Matthysse et al., supra; de Lorenzo et al., supra; Herrero et al., supra; see GenBank® AY364166) was digested with XmaI at 37° C. for two hours, which was followed by a brief dephosphorylation treatment with Shrimp Alkaline Phosphatase (SAP) (USB Corporation, Cleveland, Ohio). The digestion reaction was separated on a 0.7% TBE agarose gel and the Zymo DNA extraction kit was used to purify the vector DNA fragment (Zymo Research, Orange, Calif.). This digestion resulted in the removal of the gfp and tet genes, but left intact the plasmid functions, the gene encoding the transposase, and the ends of the Tn5 transposon.

### Preparation of Multiple Cloning Site (MCS) Insert DNA

Two PCR primers MCS.F 5′-AATTCCCGGGACTAGTACGCGTGCGGCCGCCCATGGCATATGTTCG AACCCGGGTACC-3′ (SEQ ID NO: 9) and MCS.R 5′-GGTACCCGGGTTCGAACATATGCCATGGGCGGCCGCACGCGTACTA GTCCCGGGA-3′ (SEQ ID NO: 10) were annealed together under the following conditions. They were mixed together is a 1:1 molar ratio to a final concentration of 100 pmol/μL. The mixture was heated to 100° C. for five minutes, then gradually cooled over ˜20 minutes by turning off the heat source. As the temperature cooled to 40° C., the tubes were transferred to ice. The annealed primers were subsequently digested with restriction endonuclease XmaI. The QIAquick Nucleotide Removal Kit (Qiagen, Valencia, Calif.) was used to purified the MCS insert DNA.

### Construction of the pUTmTn5 Vector+Multiple Cloning Site (MCS)

The XmaI digested and SAP dephosphorylated pUTmTn5 vector DNA was ligated with the XmaI digested MCS insert DNA at 11° C. for 15 minutes. Prior to electroporation, the ligation reaction was heat inactivated by incubation at 70° C. for 5 minutes. One microliter of the ligation mixture was electroporated into 30 μL of electrocompetent E. Coli SY327 cells (Miller, V. L. and Mekalanos, J. J., Proc. Natl. Acad. Sci., 81(11):3471-3475 (1984). The cells were allowed to recover in 400 mL of SOC medium for 90 minutes and 50 μL and 100 μL was plated onto LB+ampicillin (100 μg/mL) agar plates. Twenty-four transformants were selected for plasmid isolation. The mini-prep (Qiagen) plasmid DNA was digested with SpeI/NheI at 37° C. for 1.5 hours. The plasmid DNA samples containing an insert DNA fragment produce two DNA fragments (˜1.1 kB & 4.2 kB) when digested with SpeI and NheI. One out of ten clones was correct. The orientation of the insert DNA was determined via DNA sequencing using two DNA sequencing primers pUTmTn5/Seq.F 5′-GCACGATGAAGAGCAGAAGTTATC-3′ (SEQ ID NO: 11) and pUTmTn5/Seq.R 5′-AACACTTAACGGCTGACATGG-3′ (SEQ ID NO: 12).

### Construction of the pUTmTn5-334

### Promoterless Astaxanthin Transposon

The astaxanthin-producing plasmid pDCQ334 (SEQ ID NO: 1) was the source of carotenoid genes used to construct pUTmTn5-334.

The transposon vector (pUTmTn5) and pDCQ334 were both digested with BstBI and SpeI. Digestion of pDCQ334 with BstBI and SpeI liberated the entire carotenoid cluster (crtWZEidiYIB) (SEQ ID NO: 5) from pDCQ334 without any promoter sequences from the vector. The two DNA samples were incubated with BstBI at 65° C. for 2 hrs; subsequently, the two DNA samples were further digested SpeI. This digestion mixture was incubated at 37° C. for several more hours. The SpeI/BstBI digested DNA samples were separated on an agarose preparative gel. The desired bands (an ˜5.2 kB band for the insert DNA fragment containing the carotenoid genes from pDCQ334 and an ˜7.4 kB band for the pUTmTn5 vector DNA fragment) were excised from the gel and purified using the Zymo DNA extraction kit (Zymo Research Corp.). This DNA was used in the ligation reaction, which was allowed to incubate for 15 minutes at room temperature. Following the incubation period, the ligation reaction was heat inactivated by incubation at 70° C. for 15 minutes, 1 μL of the ligation mixture was electroporated into 32 μL of E. coli SY327 electroporation-competent cells. The transformed cells recovered for ˜1 hour at 37° C. in 800 μL SOC medium; next, all of the transformation mixture was spread unto LB+Amp¹⁰⁰ (100 μg/mL) plates. Ten colonies were picked and cultured overnight for plasmid DNA isolation. The plasmid DNA (Qiagen Mini-prep Kit) was digested with MfeI. In addition to identifying correct transposon clones, this digestion would also allow the orientation of the MCS to be confirmed. The expected size of the DNA fragments were ˜9.2 kB & 3.4 kB if the MCS were in the (+) orientation and ˜8.1 kB & 4.5 kB if the MCS were in the (−) orientation. All ten of the pUTmTn5-334 candidates produced two DNA fragments that were ˜8.1 kB and 4.5 kB in size, indicating that the correct insert DNA fragment was ligated into the pUTmTn5 transposon vector and that the MCS was in the negative orientation. The next step in the construction of the transposon vector is the addition of an antibiotic resistance gene, which permits the transconjugants to be isolated following the conjugation reaction.

### Construction of the pUTmTn5-334Kn

### Promoterless Astaxanthin Transposon

To select for transconjugants that received a transposon insertion during the conjugation, the antibiotic resistance gene that confers resistance to kanamycin was inserted between the transposon ends. The source of the kanamycin resistance gene was transposon EZ::TN™ <Kan-2> (Epicentre, Madison, Wis.). PCR amplification of the EZ::TN™ <Kan-2> kanamycin resistance gene was accomplished using PCR primers KnAvrIIKpnIBstBI.R2 5′-ATGCTTCGAACGGGTACCTAGGATGCGTGATCTGATCC-3′ (SEQ ID NO: 13) and KnBstBI.F 5′-TGGCTTCGAACGATGAATTGTGTCTC-3′ (SEQ ID NO: 14) using the following PCR program: Hold (94° C., 4 min.); 20 cycles (93° C., 30 sec; 50-60° C. gradient, 1 min.; 72° C., 1.5 min.); Hold (72° C., 1.5 min.); Hold (4° C.). After visualizing the product(s) of the PCR reaction on an agarose gel, 0.5 μL of the PCR product was used as the insert DNA in a TOPO ligation reaction in which pCR®2.1 was the vector DNA (TA Cloning® Kit, Invitrogen, Carlsbad, Calif.). The ligation reaction incubated at room temperature for 5 minutes and was used to transform chemically competent E. coli One Shot® TOP10 cells according to Invitrogen's protocol. Five white colonies from Blue/White screen were cultivated for plasmid DNA isolation (Qiagen Plasmid Mini Kit). Digestion of the plasmid DNA with XhoI and visualization on a 0.7% agarose gel revealed that all five candidates were correct and were ligated in the reverse orientation. The plasmid was designated pCR2.1Kn^{R}. In preparation for the ligation reaction, a larger quantity of pCR2.1 Kn^{R} and pUTmTn5-334 plasmid DNA was sequentially digested with BstBI and AvrII. First the BstBI restriction digestion reaction was carried out at 65° C. for two hours, next the temperature was cooled to 37° C. and AvrII was added and the reaction continued for an additional two hours. The vector DNA was dephosphorylated to prevent vector re-ligation using SAP by incubation at 37° C. for 1 hour. The fragments for the insert DNA were separated on an agarose gel, an ˜1 kB DNA fragment was excised and purified using the Zymo DNA extraction kit (Zymo Research). The BstBI and AvrII digested vector and insert DNA were ligated for 15 minutes at room temperature, afterward the reaction was heat-inactivated at 70° C. for 15 minutes and 0.5 μL of the ligation reaction was used to transform 40 μL of E. coli SY327 cells. Following incubation on ice and heat shock, 800 μL of SOC medium was added and the cells were allowed to recover at 37° C. for 1 hour. Approximately 50 μL of transformation mixture was plated onto LB+Kn²⁵ agar plates. Ten colonies were patched onto LB+Kn²⁵ plates; two of the patches were selected for plasmid isolation (Qiagen Plasmid Mini Prep Kit). The pUTmTn5-334Kn candidates were confirmed to be correct by digestion with XhoI and NotI. Three DNA fragments (˜9.3 kB, 3.0 kB & 1.4 kB in size) were generated for both candidate plasmids. The transposon vector pUTmTn5-334Kn will be conjugated into Methylomonas to identify chromosomal locations that support high-level carotenoid synthesis.

### Construction of the pUTmTn5-343

### Promoterless Astaxanthin Transposon

The astaxanthin-producing plasmid pDCQ343 (SEQ ID NO: 3) was prepared by cloning into plasmid pDCQ341 the Brevundimonas vesicularis DC263 crtZ hydroxylase coding region (SEQ ID NO: 16; U.S. 60/601,947) into the crtWEYIB gene cluster forming a crtWZEYIB carotenoid gene cluster (SEQ ID NO: 7) operably linked to the P_{cat} promoter. Plasmid pDCQ343 was the source of carotenoid genes used to construct pUTmTn5-343.

The transposon vector (pUTmTn5) and pDCQ343 were both digested with BstBI and SpeI. Digestion of pDCQ343 with BstBI and SpeI liberated the backbone carotenoid genes (crtE, crtY, crtI, and crtB) from pDCQ343 without any promoter sequences from the vector. The two DNA samples were incubated with BstBI at 65° C. for 2 hrs; subsequently, the two DNA samples were further digested SpeI. This digestion mixture was incubated at 37° C. for several more hours. The SpeI/BstBI digested DNA samples were separated on an agarose preparative gel. The desired bands (an ˜4.2 kB band for the insert DNA fragment containing the carotenoid genes from pDCQ343 and an ˜7.4 kB band for the pUTmTn5 vector DNA fragment) were excised from the gel and purified using the Zymo DNA extraction kit. This DNA was used in the ligation reaction, which was allowed to incubate for 15 minutes at room temperature. Following the incubation period, the ligation reaction was heat inactivated by incubation at 70° C. for 15 minutes, 1 μL of the ligation mixture was electroporated into 32 μL of E. coli SY327 electroporation-competent cells. The transformed cells recovered for ˜1 hour at 37° C. in 800 μL SOC medium; next, all of the transformation mixture was spread on to LB+Amp¹⁰⁰ plates. Five colonies were picked and cultured overnight for plasmid DNA isolation. The plasmid DNA (Qiagen Mini-prep Kit) was digested with MfeI. In addition to identifying correct transposon clones, this digestion would also allow the orientation of the MCS to be confirmed. The expected size of the DNA fragments were ˜9.0 kB & 1.3 kB if the MCS were in the (+) orientation and ˜6.0 kB & 4.3 kB if the MCS were in the (−) orientation. Four of the five pUTmTn5-343 candidates produced two DNA fragments that were ˜8.1 kB and 4.5 kB in size, indicating that the correct insert DNA fragment was ligated into the pUTmTn5 transposon vector and that the MCS was in the negative orientation.

The addition of the crtW and crtZ genes as well as an antibiotic resistance gene to the pUTmTn5-343EYIB vector was still required to allow the transposon vector use in the identification of chromosomal locations that support high-level production of astaxanthin.

The crtW (SEQ ID NO: 15) and crtZ (SEQ ID NO: 16) genes were amplified from pDCQ343 template DNA using PCR primers p343crtZSpeI.F 5′-TACCCACTAGTAAGGAGGAATAAACCATGACCG-3′ (SEQ ID NO: 17) and p343crtWSpeI.R 5′-GGTTGGTACTAGTTCAGGC-3′ (SEQ ID NO: 18) using the following PCR program: Hold (94° C., 4 min.); 20 cycles (94° C., 30 sec; 45-55° C. gradient, 1 min.; 72° C., 1.5 min.); Hold (72° C., 7 min.); Hold (4° C.). The PCR product was ligated into the TOPO vector pCR®2.1 and transformed into chemically competent E. coli One Shot® TOP10 cells (Invitrogen). Two white colonies from the Blue/White screen were chosen for plasmid isolation. In addition to the isolated TOPO plasmid DNA, the vector pUTmTn5-343EYIB was also digested with SpeI for three hours at 37° C. DNA fragments of the correct sizes [insert DNA (1.3 kB) and vector DNA (10.3 kB)] were excised from the agarose gel and purified using the Zymo DNA extraction kit. The purified DNA fragments (the crtWZ insert DNA and the pUTmTn5-343EYIB vector DNA) were used in the ligation reaction. The ligation of the two DNA fragments was allowed to occur for 5 minutes at room temperature. Afterward, the ligation reaction was heat inactivated by incubation at 70° C. for 15 minutes and was used to transform 40 μL of E. coli SY327 electroporation-competent cells. Following the heat shock at 42° C., the transformation mixture was allowed to recover in 800 μL SOC for 1 hour at 37° C. and was plated on LB+Amp¹⁰⁰ agar plates. Approximately 40 colonies were cultivated and the plasmid DNA was isolated using the Qiagen plasmid Mini Kit. Interestingly, one of the colonies had a slight yellowish pigment. The 40 candidates were screened for those having the correct insert DNA fragment by digestion with BsrGI and NcoI. Plasmid candidates clones containing the crtW/crtZ insert DNA fragment produced four DNA fragments (˜6.0 kB, 3.6 kB, 1.2 kB & 0.8 kB) upon digestion with BsrGI and NcoI. Three of the candidates produced DNA fragments of the correct size, which included the plasmid DNA isolated from the colony having the yellowish pigment in E. coli. These candidate clones were confirmed to have the correct insert DNA by digestion with BamHI and BsrGI. This plasmid is referred to as pUTmTn5-343.

### Construction of the pUTmTn5-343 Cm

### Promoterless Astaxanthin Transposon

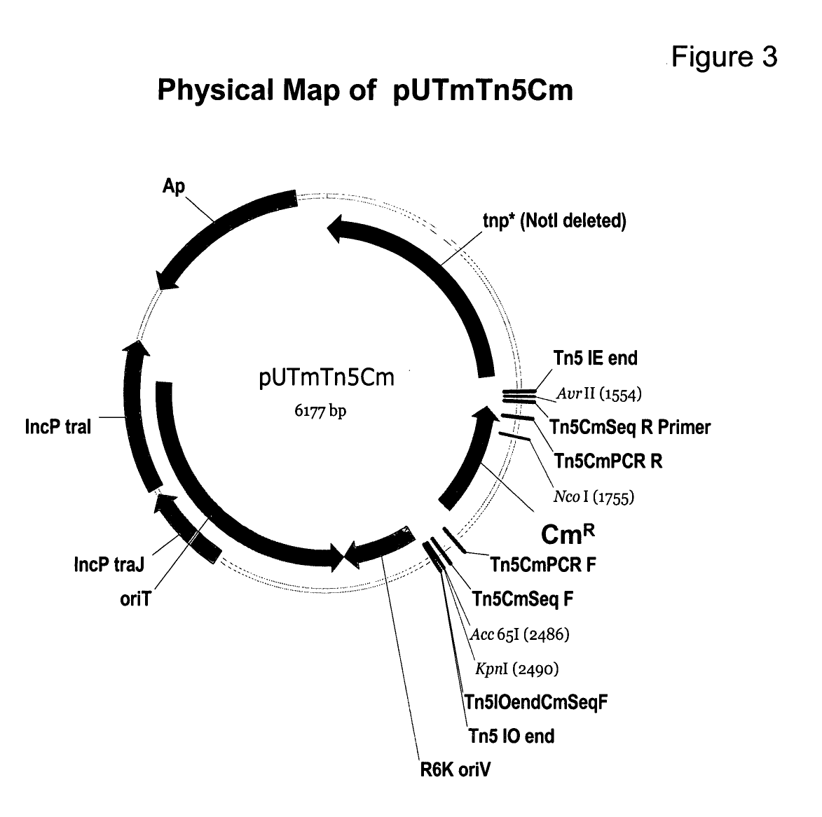

To select for transconjugants that received a transposon insertion during the conjugation, the antibiotic resistance gene that confers resistance to chloramphenicol (Cm) was inserted adjacent to the carotenoid genes for astaxanthin synthesis in pUTmTn5-343. The source of the Cm resistance gene (SEQ ID NO: 32) was pUTmTn5 Cm (FIG. 3). The transposon vector pUTmTn5 Cm was constructed by ligating an EcoRV fragment containing the gene that confers resistance to chloramphenicol from pGPS2.1 (New England Biolabs, Beverly, Mass.) into SmaI digested pUTmTn5gfptet. The genes encoding both gfp and TetA were absent from the resulting vector, pUTmTn5 Cm. The chloramphenicol resistance gene was PCR amplified using PCR primers CmAvrIIKpnIBstBI.R 5′-ATGCTTCGAACGGGTACCTAGGCGTTTAAGGGCACCAATAAC-3 (SEQ ID NO: 19) and CmBstBI.F 5′-TGGCTTCGAATACCTGTGACGGAAGATC-3′ (SEQ ID NO: 20) and the following PCR program: Hold (94° C., 4 min.); 20 cycles (94° C., 30 sec; 50-60° C. gradient, 1 min.; 72° C., 1.5 min.); Hold (72° C., 7 min.); Hold (4° C.). The Cm PCR fragment was cloned into TOPO vector pCR®2.1. Using a Blue/White screen, many white colonies were identified when the transformation was plated onto LB+Amp¹⁰⁰ agar plates. Two colonies were grown for plasmid isolation (Qiagen) and the plasmid DNA was examined for the proper insert DNA fragment by digestion with NcoI (2.7 kB and 2.2 kB in one orientation or 3.1 kB and 1.8 kB in the other orientation). Both candidates contained the appropriate insert DNA fragment.

To prepare the insert DNA for ligation into pUTmTn5, pCR2.1 Cm was digested sequentially with AvrII and BstBI. The vector DNA pUTmTn5-343 was digested with the same restriction enzymes. Both plasmids were initially incubated with AvrII at 37° C. for one hour, after that, the temperature was raised to 65° C. and BstBI was added and the reaction continued for and additional two hours. For the pUTmTn5-343 vector DNA, the reaction was cooled to 37° C., the SAP was added and the dephosphorylation reaction continued for an extra hour. The dephosphorylated vector DNA was purified using the Zymo DNA extraction kit. The insert DNA was analyzed on an agarose gel, the ˜1 kB band was excised, and purified from the gel using the Zymo DNA extraction kit. The AvrII/BstBI digested Cm insert DNA and the pUTmTn5-343 vector were ligated for 15 minutes at room temperature. The reaction was heat inactivated by incubation at 70° C. for 15 minutes. Subsequently, approximately 0.5 μL of the ligation reaction was used to transform 40 μL of E. coli SY327 cells. The transformation reaction was permitted to recover in 800 μL of SOC medium for one hour and was plated onto LB+Cm²⁵ (25 μg/mL) agar plates. Three pUTmTn5-343 Cm candidates were selected be evaluated for the presence of the Cm insert DNA using digestion with NcoI and the generation of four bands (˜6.7 kB, 3.6 kB, 1.2 kB and 0.9 kB). All three candidates were correct and the new vector was named pUTmTn5-343 Cm. The transposon vector pUTmTn5-343 Cm will be used in future conjugation reactions.

### Construction of the pUTmTn5-343Kn

### Promoterless Astaxanthin Transposon

The transposon vector pUTmTn5-343Kn vector was constructed by ligating BstBI/AvrII linearized and gel purified pUTmTn5-343 vector DNA with BstBI/AvrII digested kanamycin DNA fragment from pCR® 2.1 (Invitrogen). The joining of the vector and insert DNAs was carried out using an in-gel ligation procedure. After excising the vector DNA fragment from the agarose gel, it was soaked in 40 mL of molecular biology grade H₂O for 20 minutes to dilute the Tris-Borate-EDTA (TBE) buffer present in the agarose gel slice. The water was removed and an additional 40 mL of H₂O was added and the gel soaked for five more minutes. It was important not to soak too long due to the lost of DNA due to diffusion. The agarose gel slice was removed from the water and transferred to a new tube. Approximately half of the gel slice was used in the ligation reaction. Four microliters of the ligase buffer (IX concentration) and 2 μL of ATP was added to the agarose gel slice. The components were crushed and mixed using a pipette tip. The mixture was allowed to equilibrate for ˜30 minutes, which permitted the vector DNA to emerge from the agarose gel into the liquid and the ligation buffer components to diffuse into the pieces of agarose gel, resulting in a 1×final concentration. The in-gel ligation and standard ligation mixtures were diluted 1:3 and used to transform E. coli SY327 electroporation competent cells. The transformation mixture was plated onto LB+Kan⁵⁰ agar plates.

PCR amplification was used to screen the transformants for cells containing the correct vector DNA. The PCR primers used in the reaction was pUTmTn5/Seq.F (SEQ ID NO: 11) and KnBstBI.F (SEQ ID NO: 14). The vector pUTmTn-334Kn was also amplified as a control. Following the PCR amplification reaction, the candidate PCR DNA, as well as, pUTmTn5-343 Cm and pUTmTn5-343 were digested with NcoI. The expected sizes of the DNA fragments pUTmTn5-343 Cm (0.95 kB, 1.2 kB, 0.36 kB, and 0.67 kB), pUTmTn5-343 (1.2 kB, 0.36 kB, and 0.67 kB), and pUTmTn5-343Kn (1.2 kB, 3.6 kB, 7.8 kB). The candidate DNA gave DNA fragments of the correct size (the 0.95 kB DNA fragment disappeared an the largest DNA fragment shifted upward). Thus, it was confirmed that the antibiotic resistance gene of pUTmTn5-343 Cm was changed from Cm to Kn, forming a plasmid referred to pUTmTn5-343Kn.

### Construction of pUTmTn5-341 Kn

### Promoterless Canthaxanthin Transposon

The transposon vector pUTmTn5-341 Kn vector was constructed by eliminating the crtZ gene from pUTmTn5-343Kn. This was accomplished by digesting pDCQ341 and pUTmTn5-343Kn with BsrG1 and AstII, which generated DNA fragments that were ˜2.9 kB and ˜9.2 kB, respectively. The gel-purified transposon vector backbone DNA from pUTmTn5-343Kn (contained a partial crtW, a partial crtI, an intact crtB, and an intact Kn^{R} gene) and the insert DNA from pDCQ341 (contained a partial crtW, the remainder of crtI, an intact crtE, and intact crtY) were joined together in a ligation reaction. After terminating the ligation reaction by heating at 70° C. overnight, 0.5 mL of the ligation mixture was used to transform electroporation competent E. coli SY327 cells. The electroporation mixture recovered for one hour in 800 mL of SOC medium and was plated onto LB+Amp⁵⁰ agar plates. PCR amplification using isolated colonies as the DNA source was used to screen for colonies containing the correct insert DNA fragment using PCR primers pUTmTn5Seq.R (5′-AACACTTAACGGCTGACATGG-3′)(SEQ ID NO: 12) and crtE343R (5-ACATCGTATTGCGTGCGCAT-3′) (SEQ ID NO: 21) and the following PCR parameters: Hold (94° C. for 4 min.); 30 cycles (94° C. for 30 sec., 52° C. for 30 sec., 72° C. for 2.5 min.); Hold (72° C. for 10 min.); Hold (4° C.). Unfortunately, the PCR results were ambiguous, therefore, colonies were streaked onto agar plates and these cells were used for mini-prep DNA isolation. The plasmid DNA was isolated from four colonies and was digested with SpeI. The expected DNA fragment sizes were ˜11.4 kB & ˜1.3 kB for the parental vector pUTmTn5-343Kn and ˜11.4 kB & 0.8 kB for the new transposon vector pUTmTn5-341 Kn. One of the four samples had the correct insert DNA. It was also noticed that cells from this sample were slightly yellow in color, suggesting that the promoterless carotenoid transposon genes were being expressed from a remote promoter in the vector sequences.

### Construction of the pUTmTn5-377Kn

### Promoterless Astaxanthin Transposon

The transposon vector pUTmTn5-377Kn was constructed by removing the carotenoid gene cluster form pUTmTn5-334Kn (Example 1) and replacing it with the carotenoid gene cluster from pDCQ377 (SEQ ID NO: 4).

The carotenoid cluster in pDCQ334 was released from the vector backbone using BstBI and XmaI. This digestion was carried out in two steps. First, the DNA is digested XmaI for two hours at 37° C., subsequently the temperature is raised to 65° C., BstBI is added and the reaction proceeded for an additional two hours. Upon completion of the digestion reaction, the DNA fragments were dephosphorylated with SAP to prevent re-ligation of the vector during the ligation reaction. There were five bands (6.3 kB, 4.3 kB. 2.5 kB 0.3 kB & 0.2 kB) generated during the digestion. It as very important that the digestion reaction went to completion, so that the smaller DNA fragments (0.3 kB & 0.2 kB) were liberated from the desired 6.3 kb DNA fragment which contained the element necessary for vector replication, conjugation and transposition. The 6.3 kB DNA fragment was excised from the agarose gel and the DNA was extracted using the Zymo DNA extraction kit.

The carotenoid gene cluster in pDCQ377 (SEQ ID NO: 4) was removed using BspEI and BstBI. Since the two enzymes use different buffers the reaction was performed in two steps. First, the DNA was digested with BspEI at 37° C. for two hours. Afterward the salt from the digestion reaction was removed using columns from the Zymo DNA extraction kit. Next, BstBI was added to the DNA, which incubated at 65° C. for an additional hour. There were two bands (7.5 kB & 4.8 kB) generated. The 7.5 kB DNA fragment, which contained the carotenoid gene cluster necessary for the production of astaxanthin, was excised from the agarose gel and purified using the Zymo kit. The ligation of the carotenoid gene cluster from pDCQ377 into the pUTmTn5Kn vector was not successful after multiple attempts. Therefore, a new cloning strategy was designed in which pUTmTn5-334 was digested with XmaI and NcoI and pDCQ377 was digested with BspEI and NcoI. Subsequently, the pDCQ377 digested DNA was dephosphorylated using SAP. A DNA fragment ˜6.5 kB in size was excised from the gel for the pUTmTn5-334Kn digested DNA and a DNA fragment ˜7.2 kB was cut from the gel of the pDCQ377 digested DNA. Following the clean up of the DNA samples using the Montage Kit, the insert and vector DNA fragments were used in the ligation reaction, which incubated for 20 minutes at room temperature. The ligation reaction was heat inactivated at 70° C. for 15 minutes prior to the transformation of 50 μL of electroporation-competent E. coli SY327 cells. After incubation on ice and the heat shock reaction, the transformation recovered in 800 μL of SOC medium for ˜45 minutes and was plated onto LB+Amp¹⁰⁰ agar plates. Fourteen colonies were picked for plasmid DNA purification (Qiagen). The 14 candidate plasmids were screened by digestion with SpeI/NheI/XbaI; one of the candidate plasmids exhibited the correct restriction pattern. The candidate was confirmed by digestion with KpnI, which generated four DNA fragments (˜11.0 kB, 1.3 kB, 1.0 kB & 0.5 kB). The new vector was named pUTmTn5-377Kn. The transposon vector pUTmTn5-377Kn will be used in future conjugation reactions.

### Example 2

### Growth Of Methylomonas Sp. 16A

Example 2 describes the standard conditions used for growth of Methylomonas sp. 16a (ATCC PTA-2402) and derivatives thereof, as described in U.S. Pat. No.6,689,601, hereby incorporated by reference.

Methylomonas Strain and Culture Media

The growth conditions described below were used throughout the following experimental Examples for treatment of Methylomonas sp., unless conditions were specifically described otherwise.

Briefly, Methylomonas sp. MWM1200 was typically grown in serum stoppered Wheaton bottles (Wheaton Scientific; Wheaton, Ill.) using a gas/liquid ratio of at least 8:1 (i.e., 20 mL or less of ammonium liquid “BTZ” growth medium in a Wheaton bottle of 160 mL total volume). The composition of the BTZ growth medium is given below. The standard gas phase for cultivation contained 25% methane in air, although methane concentrations can vary ranging from about 5-50% by volume of the culture headspace. These conditions comprise growth conditions and the cells are referred to as growing cells. In all cases, the cultures were grown at 30° C. with constant shaking in a rotary shaker (Lab-Line, Barnstead/Thermolyne; Dubuque, Iowa) unless otherwise specified.

BTZ Media for Methylomonas sp.

Methylomonas 16a and derivatives thereof (e.g. Methylomonas sp. MWM1200) typically grows in a defined medium composed of only minimal salts; no organic additions such as yeast extract or vitamins are required to achieve growth. This defined medium known as BTZ medium (also referred to herein as “ammonium liquid medium”) consisted of various salts mixed with Solution 1, as indicated in Tables 1 and 2. Alternatively, the ammonium chloride was replaced with 10 mM sodium nitrate to give “BTZ (nitrate) medium”, where specified. Solution 1 provides the composition for a 100-fold concentrated stock solution of trace minerals.

TABLE 1
Solution 1*

| | Molecular | Conc. | |
|---|---|---|---|
| | Weight | (mM) | g per L |
| Nitriloacetic acid | 191.10 | 66.90 | 12.80 |
| CuCl₂ × 2H₂O | 170.48 | 0.15 | 0.0254 |
| FeCl₂ × 4H₂O | 198.81 | 1.50 | 0.30 |
| MnCl₂ × 4H₂O | 197.91 | 0.50 | 0.10 |
| CoCl₂ × 6H₂O | 237.90 | 1.31 | 0.312 |
| ZnCl₂ | 136.29 | 0.73 | 0.10 |
| H₃BO₃ | 61.83 | 0.16 | 0.01 |
| Na₂MoO₄ × 2H₂O | 241.95 | 0.04 | 0.01 |
| NiCl₂ × 6H₂O | 237.70 | 0.77 | 0.184 |

*Mix the gram amounts designated above in 900 mL of H₂O, adjust to pH = 7.0, and add H₂O to a final volume of 1 L. Keep refrigerated.

TABLE 2
Ammonium Liquid Medium (BTZ)**

| | | Conc. | |
|---|---|---|---|
| | MW | (mM) | g per L |
| NH₄Cl | 53.49 | 10 | 0.537 |
| KH₂PO₄ | 136.09 | 3.67 | 0.5 |
| Na₂SO₄ | 142.04 | 3.52 | 0.5 |
| MgCl₂ × 6H₂O | 203.3 | 0.98 | 0.2 |
| CaCl₂ × 2H₂O | 147.02 | 0.68 | 0.1 |
| 1 M HEPES (pH 7.0) | 238.3 | | 50 ml |
| Solution 1 | | | 10 ml |

**Dissolve in 900 mL H₂O. Adjust to pH = 7.0, and add H₂O to give a final volume of 1 L. For agar plates: Add 15 g of agarose in 1 L of medium, autoclave, cool liquid solution to 50° C., mix, and pour plates.

Plates were incubated in a closed jar with 25% methane at 30° C.

### Example 3

### Tri-Parental Conjugation of the Various Transposon Vectors into Methylomonas sp

The genetic procedure of in vivo transposition was used to screen the Methylomonas genome for chromosomal locations that will support high-level carotenoid expression. The first promoterless carotenoid transposon used was Tn334Kn. Several colonies were identified that exhibited a high level of total carotenoid production.

Each of the promoterless carotenoid transposon vectors were transferred into Methylomonas sp. MWM1200 via triparental conjugation. Specifically, the following were used as recipient, donor, and helper, respectively: Methylomonas sp. MWM1200, E. coli SY327 containing the promoterless carotenoid transposon vectors, and E. coli containing pRK2013 (ATCC No. 37159).

Theory of the Conjugation and In Vivo Transposition

The mobilization of vector DNA into Methylomonas occurs through conjugation (tri-parental mating)(see U.S. Ser. No. 10/997,308, U.S. Ser. No. 10/997,844, and U.S. Ser. No. 11/070,080; hereby incorporated by reference). The pGP704-derived vector used to make transposon insertions into Methylomonas genome has a R6K origin of replication, which requires the Π protein. This vector can replicate in E. coli strain SY327, which expresses the Π protein. However, this protein is not present in the Methylomonas genome. Therefore, once the vector DNA has entered into Methylomonas, it is unable to duplicate itself. The transposase, the enzyme responsible for the mobilization of the transposon, is located outside of the transposon ends. Therefore, once the carotenoid transposon inserts into the Methylomonas genome, the gene(s) contained between the transposon ends are unable to move a second time within the Methylomonas genome.

In the case of Methylomonas, the transposon plasmids were used to transfer the promoterless carotenoid transposon into this bacterium. The conjugative plasmid (pRK2013; ATCC No. 37159), which resided in a strain of E. coli, facilitated the DNA transfer.

Growth of Methylomonas sp.

The growth of Methylomonas sp. or tri-parental mating initiated with the inoculation of fresh Methylomonas cells into 20 mL of BTZ medium containing 25% methane. The culture was grown at 30° C. with aeration until the density of the culture was saturated producing the seed culture. This seed culture was in turn used to inoculate two bottles containing 100 mL of fresh BTZ medium containing 25% methane. These bottles were inoculated with 200 μL and 400 μL of the seed culture. The following day the two cultures were diluted 1:5 into fresh BTZ medium and were grown at 30° C. with aeration until the culture reached an OD₆₀₀ between 0.7 to 0.9. The bottles having an OD₆₀₀ closest to the target OD were used in the conjugation. To prepare the cells for the tri-parental mating, the Methylomonas sp. 16a cells were washed twice in an equal volume of BTZ medium. The Methylomonas cell pellets were re-suspended in the minimal volume needed (approximately 250 to 350 μL). Approximately 60 μL of the re-suspended Methylomonas cells were used in each tri-parental mating experiment.

Growth of the Escherichia coli Donor and Helper Cells

Isolated colonies of the E. coli donor (containing a transposon vector comprising a promoterless carotenoid gene cluster) and helper (containing conjugative plasmid pRK2013) cells were used to inoculate 5 mL of LB broth containing 25 μg/μL Kan; these cultures were grown overnight at 30° C. with aeration. The following day, the E. coli donor and helper cells were washed twice in equal volumes of fresh LB broth to remove the antibiotics and combined together in the same test tube.

Tri-Parental Mating: Mobilization of the Donor Plasmid into Methylomonas sp. 16a

Approximately 60 μL of the re-suspended Methylomonas cells were used to re-suspend the combined E. coli donor and helper cell pellets. After thoroughly mixing the cells, the cell suspension was spotted onto BTZ agar plates containing 0.05% yeast extract. The plates were incubated at 30° C. for 3 days in a jar containing 25% methane.

Following the third day of incubation, the cells were scraped from the plate and re-suspended in BTZ broth. The entire cell suspension was plated onto several BTZ agar plates containing Kan⁵⁰. The plates were incubated at 30° C. in a jar containing 25% methane until colonies were visible (˜4-7 days).

Approximately twenty colonies were streaked in quadrants onto fresh BTZ+Kan⁵⁰ agar plates and incubated 1-2 days at 30° C. in the presence of 25% methane. These cells were used to inoculate bottles containing 20 mL of BTZ and 25% methane. After overnight growth, 5 mL of the culture was concentrated by centrifugation using a tabletop centrifuge. Then, to rid the cultures of E. coli cells that were introduced during the tri-parental mating, the cells were inoculated into 20 mL of BTZ liquid medium containing nitrate (10 mM) as the nitrogen source, methanol (200 mM), and 25% methane and grown overnight at 30° C. with aeration. Cells from the BTZ (nitrate) cultures were again inoculated into BTZ and 25% methane and grown overnight at 30° C. with aeration. The cultures were monitored for E. coli growth by plating onto LB agar plates to verify the success of the E. coli elimination.

### Example 4

### Identification of Chromosomal Insertion Sites for the Promoterless Carotenoid Transposons

Two different approaches were used to determine the location of the transposons (e.g. Tn5-334Kn transposon) within the Methylomonas genome. A single primer PCR method was used to amplify regions of the Methylomonas chromosome (Karlyshev et. al., Biotechniques June 28(6) 1078-82 (2000)). The single primer PCR method required a nested set of primers be designed at both transposon ends. One set of primers was used in the PCR amplification reaction and the other primer set was used in the sequencing reactions. The other method involved direct sequencing of Methylomonas chromosomal DNA using DNA primers specific for the end of the transposable element. The insertion sites of the transposable elements are shown in FIG. 5.

The single primer PCR method required the amplification of PCR products from the Methylomonas chromosomal DNA using the following PCR reaction mixture (50 mL total volume): 19.75 μL H₂O, 5.0 μL 10×PCR buffer, 4.0 μL MgCl₂, 15.0 μL Enhancer, 5.0 μL dNTP's (2 mM), 0.5 μL PCR primer (100 μM), 0.25 μL Taq DNA polymerase, & 0.5 μL DNA (Methylomonas cells). The PCR primers used for the amplification of the transposon:chromosome junctions are listed in Table 3 (Primers A & C were used to determine the insertion sites of the Tn5-334Kn transposon, primers E & G were used to determine the insertion sites of the Tn5-343 Cm and Tn5-341 Kn transposons, and primers I & C were used to determine the insertion sites of the Tn5-377Kn transposon) and the thermocycling parameters were:

| | | |
|---|---|---|
| 1 | cycle | 5 min. 94° C. |
| 20 | cycles | 30 sec. 94° C., 30 sec. 60° C., 3 min. 72° C. |
| 30 | cycles | 30 sec. 94° C., 30 sec. 40° C., 2 min. 72° C. |
| 30 | cycles | 30 sec. 94° C., 30 sec. 60° C., 2 min. 72° C. |
| 1 | cycle | 7 min 72° C. |

| | |
|---|---|
| Hold | 4° C. |

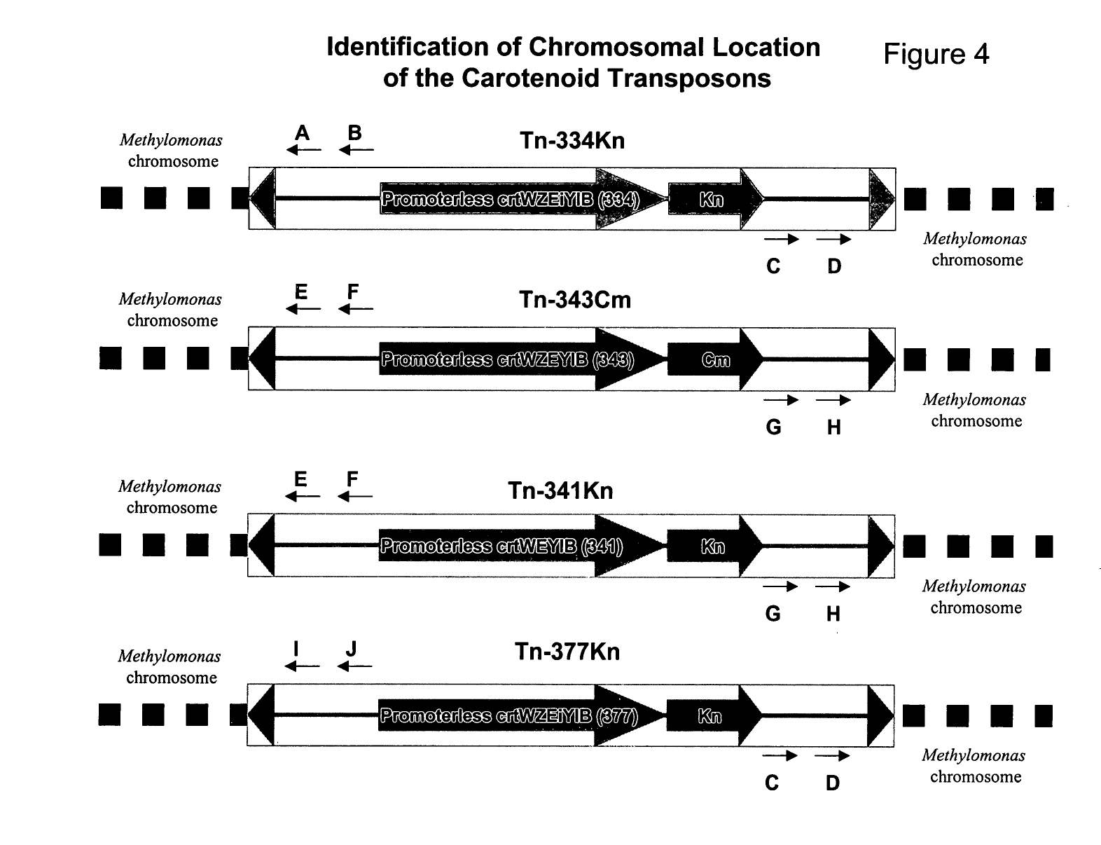

The sequencing primers used to determine the chromosomal locations of the carotenoid transposons are shown in Table 3. Sequencing Primer B was used to sequence the Primer A PCR product for the Tn5-334Kn insertion sites (FIG. 4). Sequencing Primer D was used to sequence to the Primer C PCR product for the Tn5-334Kn and the Tn5-334Kn insertion sites. Sequencing Primer F was used to sequence the Primer E PCR product for the Tn5-343 Cm and Tn5-341 Kn insertion sites. Sequencing Primer H was used to sequence the Primer G PCR product for the Tn5-343 Cm and Tn5-341 Kn insertion sites. Sequencing Primer J was used to sequence the Primer I PCR product for the Tn5-377Kn insertion sites. Following PCR amplification of the transposon insertion region via single primer PCR, the Qiagen 96-well PCR cleanup kit was used to remove the PCR primer prior to submission of the PCR fragments for DNA sequencing. The Sequencing primer, which also bound the transposon end, was used to sequence the PCR fragment. This sequence information was used to determine the transposon-chromosome junction site.

Chromosomal DNA (from strains MCIS2201 [fliS] and MCIS2203 [fliC]) was isolated from 0.5 mL of dense Methylomonas culture (OD ˜3.5) using the Epicentre MasterPure™ DNA Purification Kit according to manufacturers directions (Epicenter Technologies). The final DNA pellet was resuspended in 100 μL EB (Tris 10 mM, pH 8.5) and used undiluted for direct sequencing of chromosomal templates. The recommended DNA concentration for this procedure is 200-500 μg/μL. Primers were diluted to 10 pmol/μL in H₂O. Four primers were used on each of the two templates. Primer sequences are shown in Table 3 (see FIG. 4).

TABLE 3
Primer Sequences for DNA Sequencing

| | | | |
|---|---|---|---|
| Primer | Primer Name | Length | DNA Sequence |

| | | | |
|---|---|---|---|
| A | pUTmTn5-334KnPCR.F | 24 | 5′-GAACCACAGGGCATGGACATGCAG-3′ |
| | | | (SEQ ID NO:22) |
| B | pUTmTn5-334KnSeq.F | 22 | 5′-GGGCGCTCATGGTTTATTCCTC-3′ |
| | | | (SEQ ID NO:24) |
| C | pUTmTn5-334KnPCR.R | 25 | 5′-GCAGTTTCATTTGATGCTCGATGAG-3′ |
| | | | (SEQ ID NO:23) |
| D | pUTmTn5-334KnSeq.R | 27 | 5′-GGGACGGCGGCTTTGTTGAATAAATCG-3′ |
| | | | (SEQ ID NO:25) |
| E | pUTmTn5-343CmPCR.F | 19 | 5′-GACATGGATCGCCAGCCAC-3′ |
| | | | (SEQ ID NO:26) |
| F | pUTmTn5-343CmSeq.F | 20 | 5′-GTCGTGATCGACGGTCATGG-3′ |
| | | | (SEQ ID NO:27) |
| G | pUTmTn5-343CmPCR.R | 27 | 5′-CCAGACCGTTCAGCTGGATATTACGGC-3′ |
| | | | (SEQ ID NO:28) |
| H | pUTmTn5-343CmSeq.R | 25 | 5′-AGGCGGCCAGATCTGATCAAGAGAC-3′ |
| | | | (SEQ ID NO:29) |
| I | pUTmTn5-377KnPCR.F | 23 | 5′-GTTCGGGACGACCCGTGACATTG-3′ |
| | | | (SEQ ID NO:30) |
| J | pUTmTn5-377KnSeq.F | 23 | 5′-CATGGCGCCGACACTTAGCGCATC-3′ |
| | | | (SEQ ID NO:31) |

Sequencing reactions for three of the four primers resulted in useful data for MCIS2201 and sequencing reactions from two of the four primers resulted in useful data for MCIS2203 (Table 5). Sequence data in both directions agree upon the chromosomal location for both templates; this provides evidence that the identified locations are accurate. Methylomonas canthaxanthin-producing strains MCIS2201 and MCIS2203 were demonstrated to contain carotenoid transposons inserted into the fliS and fliC genes, respectively.

### Example 5

### Genes within the Identified Integration Site

Numerous open reading frames were identified upon sequencing the regions flanking the transposon insertion sites. BLASTX analysis was used to identify the closest matching sequence in GenBank®. The results of BLASTX analysis are provided in Table 4.

TABLE 4
Top BLASTX Hits for the Open Reading Frames Identified in the fliC Chromosomal Region of Methylomonas sp. 16a

| ORF | Gene | Similarity Identified | SEQ ID | SEQ ID | % | % | | |
|---|---|---|---|---|---|---|---|---|
| Name | Name | GenBank ® Identification Nos. | Nucleotide | Peptide | Identity ^{a} | Similarity ^{b} | E-value ^{c} | Citation |
| 1 | fliC | Flagellin protein | 35 | 36 | 53 | 66 | 1e−72 | Direct |
| | | ZP_00173465.2 GI: 53760270 | | | | | | submission |
| | | Methylobacillus flagellatus KT | | | | | | |
| 2 | flaG | Flagellin FlaG | 37 | 38 | 41 | 66 | 2e−11 | Methe et al., |
| | | gi\|3999810\|ref\|NP_954057.1 | | | | | | Science 302 (5652), |
| | | Geobacter sulfurreducens PCA | | | | | | 1967-1969 (2003) |
| 3 | fliD | Flagellar hook-associated protein | 39 | 40 | 33 | 54 | 1e−66 | Chain et al., J. |
| | | FliD | | | | | | Bacteriol. 185: |
| | | NP_841634.1 GI: 30249564 | | | | | | 2759-2773 (2003) |
| | | Nitrosomonas europaea ATCC | | | | | | |
| | | 19718 | | | | | | |
| 4 | fliS | Flagellin-specific chaperone FliS | 41 | 42 | 49 | 69 | 3e−26 | Direct |
| | | ZP_00138683.1:GI: 32040668 | | | | | | submission |
| | | Pseudomonas aeruginosa | | | | | | |
| | | UCBPP-PA14 | | | | | | |

^{a} % Identity is defined as percentage of amino acids that are identical between the two proteins.
^{b} % Similarity is defined as percentage of amino acids that are identical or conserved between the two proteins.
^{c} Expect value. The Expect value estimates the statistical significance of the match, specifying the number of matches, with a given score, that are expected in a search of a database of this size absolutely by chance.

### Example 6

### Evaluation of Total Carotenoid Titers in Methylomonas Astaxanthin-Transposon Insertion Mutants

The carotenoid titers were calculated by determining the amount of carotenoid (milligrams) per dry cell weight [DCW] (kilogram). After cultivating the Methylomonas astaxanthin or canthaxanthin-producing strains in 50 mL of BTZ medium, 20 mL of the culture was used for carotenoid extraction and 20 mL of the culture was used to determine DCW.

For the extraction of carotenoids, the cells were pelleted in a 50 mL polypropylene tube. Following the removable of the supernatant (growth medium), approximately 0.5 mL of 0.1 mm glass beads were added to the pellet. To this mixture, 1 mL of ethanol and 1.5 mL of dichloromethane was added and the mixture was vortexed for approximately two minutes (until the cells were broken). The cellular debris was removed by centrifugation at 8000 rpm for 10 minutes. The supernatant was transferred to a new 50 mL polypropylene tube and the extracted carotenoids were dried under nitrogen for approximately two hours (until all liquid had evaporated. The dried pellets were resuspended in 90 μL of chloroform plus 1910 μL of hexane. The solution was filtered using a 0.2 μm Teflon filter (Pall Gelman Acrodisc 13 CR, PTFE syringe filter) to remove the large particles. The filtered carotenoid solution was analyzed via High Pressure Liquid Chromatography (HPLC).

To determine DCW for the Methylomonas carotenoid-producing strains, filtration was employed. Using the house vacuum, the cultures were applied to a 47 mm, 300 mL capacity, magnetic filter funnel (Pall Gelman, Ann Arbor, Mich.). A polypropylene separator [47 mm and 10.0 μm] (Pall Gelman, Ann Arbor, Mich.) was used in conjunction with a polycarbonate Whatman Nucleopore Track-Etch membrane [47 mm and 0.2 μm] (Whatman, Florham, N.J.) to collect the Methylomonas cells. The vacuum was applied until no visible liquid remained. The filter was allowed to dry over-night in a 55° C. oven. The DCW was calculated by subtracting the filter alone weight from the filter plus cells weight.

Several chromosomal locations were repeatedly identified using different carotenoid gene clusters that support elevated levels of total carotenoid synthesis in Methylomonas (Table 5). The chromosomal region supporting the highest carotenoid titer was involved in flagellar biosynthesis (fliC region). Insertions into fliC and fliS resulted approximately three-fold increase in total carotenoid synthesis over the previously identified Methylomonas carotenoid strain Tig333 (a canthaxanthin producing strain; U.S. Ser. No. 11/070,080).

TABLE 5
Summary of Various Methylomonas Strains, Transposon
Insertion Sites, and Total Carotenoid Titer.

| | | | | Total |
|---|---|---|---|---|
| Methylomonas | Carotenoid | Transposon | Genomic | Carotenoid |
| Strain | Transposon | Insertion Site | Location | Titer (ppm) |
| MCIS2203 | Tn5-341 | fliC | 530575 | ˜960 |
| MCIS1802 | Tn5-334 | fliS | 533697 | ˜2000 |
| MCIS2201 | Tn5-341 | fliS | 533721 | ˜1140 |

### Example 7

### Stability Analysis of Selected Carotenoid Transposon Insertion Mutants

In addition to identifying chromosomal locations that support increased total carotenoid titers, we also evaluated stability of several of the carotenoid transposon insertion strains using serial passages of bottle cultures. Analysis of the strains after 15-20 serial passages suggest that the majority of Methylomonas strains are stable under the conditions tested. Less than one non-pigmented colony was detected at the 10-⁻⁷ dilution (Table 6).

The fliC region is comprised of coding regions for multiple genes including fliC, flaG, fliD, and fliS. Several strains were identified having an insertion within at least one of the open reading frames within the fliC region, indicating that this region is particularly suitable as a chromosomal integration site.

The Methylomonas strains in which the carotenoid (astaxanthin) gene expression was located within the fliS open reading frame resulted in slightly more white colonies than other Methylomonas strains having increased total carotenoid titers.

TABLE 6
Stability of the Identified Chromosomal
Insertion Sites in Methylomonas

| Methylomonas | | |
|---|---|---|
| Strain | Number of Passages | Number of |
| (insertion site) | (20 mL bottles) | White Colonies (10⁻⁷) |
| MCIS2203 (fliC) | 20 | ˜1 |
| MCIS1802 (fliS) | 15 | 11 |
| MCIS2201 (fliS) | 20 | ˜1 |

What is claimed is:

1. A method for stably expressing a nucleic acid molecule in a methylotrophic microorganism comprising:
a) providing a methylotrophic microorganism having an endogenous fliC genomic region;
b) providing at least one expressible nucleic acid molecule to be stably-expressed;
c) integrating the at least one nucleic acid molecule of (b) into said fliC region of said methylotrophic microorganism whereby a transformed methylotrophic microorganism is created; and
d) growing the transformed methylotrophic microorganism of (c) under conditions whereby the at least one expressible nucleic acid molecule is stably expressed.

2. A method according to claim 1 wherein the fliC genomic region is expressed under the control of a nucleic acid molecule encoding an endogenous fliC promoter selected form the group consisting of:
a) a nucleic acid molecule as represented by SEQ ID NO: 34
b) a nucleic acid molecule that hybridizes to a) under stringent hybridization conditions comprising 0.1×SSC, 0.1% SDS, 65° C. and washed with 2×SSC, 0.1% SDS followed by 0.1×SSC, 0.1% SDS at 65° C.; and
c) a nucleic acid molecule having at least 95% identity to SEQ ID NO: 34.

3. A method according to claim 2 wherein the fliC promoter is represented by SEQ ID NO: 34.

4. A method according to any one of claims 1, 2, or 3 wherein the endogenous fliC genomic region comprises a nucleic acid molecule selected from the group consisting of:
a) a nucleic acid molecule as represented by SEQ ID NO: 33;
b) a nucleic acid molecule that hybridizes to a) under stringent hybridization conditions comprising 0.1×SSC, 0.1% SDS, 65° C. and washed with 2×SSC, 0.1% SDS followed by 0.1×SSC, 0.1% SDS at 65° C.; and
c) a nucleic acid molecule having at least 95% identity to SEQ ID NO: 33.

5. A method according to claim 4 wherein the fliC region comprises a nucleic acid molecule selected from the group consisting of SEQ ID NO: 33, 35, 37, and 39.

6. A method according to any one of claims 1, 2, or 3 wherein the fliC genomic region comprises at least one nucleic acid molecule encoding an amino acid sequence having at least 95% identity to the sequence selected from the group consisting of SEQ ID NO: 36, 38, 40, and 42.

7. A method according to claim 1 wherein the fliC genomic region comprises, in a 5′ to 3′ direction, the gene cluster fliC-fla-G-fliD-fliS.

8. A method according to claim 1 wherein the at least one expressible nucleic acid molecule comprises multiple tandem genes in a single fragment.

9. A method according to claim 1 wherein the at least one expressible nucleic acid molecule is a gene.

10. A method according to claim 1 wherein the at least one nucleic acid molecule is integrated within an open reading frame selected from the group consisting of fliC and fliS.

11. A method according to claim 1 wherein the at least one expressible nucleic acid molecule is a gene encoding an enzyme selected from the group consisting of: transaldolase, fructose bisphosphate aldolase, keto deoxy phosphogluconate aldolase, phosphoglucomutase, glucose-6-phosphate isomerase, phosphofructokinase, 6-phosphogluconate dehydratase, 6-phosphogluconate-6-phosphate-1 dehydrogenase, dxs, dxr, ispA, ispD, ispE, ispF, crtE, crtX, crtY, crtI, crtB, crtZ, crtD, crtO, crtW, idi, genes encoding limonene synthase, ugp, gumD, wza, espB, espM, waaE, espV, gumH, genes encoding glycosyltransferase genes, aroG, aroB, aroQ, aroE, arok, 5-enolpyruvylshikimate-3-phosphate synthase, aroC, trpE, trpD, trpC, trpB, pheA, tyrAc, pds, phaC, phaE, efe, pdc, adh, pinene synthase, bornyl synthase, phellandrene synthase, cineole synthase, sabinene synthase, and taxadiene synthase.

12. A method according to claim 1 wherein the at least one expressible nucleic acid molecule encodes at least one enzyme in the carotenoid biosynthetic pathway.

13. A method according to claim 12 wherein the at least one at least one enzyme in the carotenoid biosynthetic pathway is selected from the group consisting of: geranylgeranyl pyrophosphate synthase, zeaxanthin glucosyl transferase; lycopene cyclase, phytoene desaturase, phytoene synthase, β-carotene hydroxylase, β-carotene ketolase and isopentenyl diphosphate isomerase.

14. A method according to claim 1 wherein methylotrophic microorganism is a methylotrophic bacteria selected from the group consisting of Methylomonas, Methylobacter, Methylococcus, Methylosinus, Methylocyctis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, and Pseudomonas.

15. A method according to claim 1 wherein the methylotrophic microorganism is a methanotrophic microorganism.

16. A method according to claim 15 wherein the methanotrophic microorganism is a high growth methanotrophic microorganism.

17. A method according to claim 16 wherein the high growth methanotrophic microorganism is a Methylomonas sp.

18. A method according to claim 17 wherein said Methylomonas sp. comprises a 16S rRNA gene as represented by SEQ ID NO: 43.

19. A method according to claim 18 wherein said Methylomonas sp. is selected from the group consisting of Methylomonas sp. 16a (ATCC PTA-2402) and Methylomonas sp. MWM1200 (ATCC PTA-6887).

20. A method for the production of a carotenoid compound comprising:
a) providing a methylotrophic microorganism comprising at least one expressible nucleic acid molecule encoding at least one carotenoid biosynthetic pathway enzyme chromosomally integrated into a fliC region;
b) contacting the methylotrophic microorganism of (a) with a carbon substrate selected from the group consisting of methane and methanol under conditions whereby said expressible nucleic acid molecule is expressed and at least one carotenoid compound is produced; and
c) optionally recovering said carotenoid compound of (b).

21. A method according to claim 20 wherein the methylotrophic microorganism is a methylotrophic bacteria selected from the group consisting of Methylomonas, Methylobacter, Methylococcus, Methylosinus, Methylocyctis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, and Pseudomonas.

22. A method according to claim 20 wherein the methylotrophic microorganism is a high growth methanotrophic microorganism.

23. A method according to claim 22 wherein the methanotrophic microorganism is a Methylomonas sp.

24. A method according to claim 23 wherein said Methylomonas sp. has a 16S rRNA gene sequence represented by SEQ ID NO: 43.

25. A method according to claim 24 wherein said Methylomonas sp. is selected from the group consisting of Methylomonas sp. 16a (ATCC PTA-2402) and Methylomonas sp. MWM1200 (ATCC PTA-6887).

26. A method according to claim 20 wherein the genes encoding the carotenoid biosynthetic pathway encode at least one enzyme selected from the group consisting of: geranylgeranyl pyrophosphate synthase, zeaxanthin glucosyl transferase; lycopene cyclase, phytoene desaturase, phytoene synthase, β-carotene hydroxylase, β-carotene ketolase and isopentenyl diphosphate isomerase.

27. A method according to claim 20 wherein said carotenoid compound is selected from the group consisting of antheraxanthin, adonixanthin, astaxanthin, canthaxanthin, capsorubrin, alpha-cryptoxanthin alpha-carotene, beta-carotene, epsilon-carotene, echinenone, gamma-carotene, zeta-carotene, alpha-cryptoxanthin, diatoxanthin, 7,8-didehydroastaxanthin, fucoxanthin, fucoxanthinol, isorenieratene, lactucaxanthin, lutein, lycopene, neoxanthin, neurosporene, hydroxyneurosporene, peridinin, phytoene, rhodopin, rhodopin glucoside, siphonaxanthin, spheroidene, spheroidenone, spirilloxanthin, uriolide, uriolide acetate, violaxanthin, zeaxanthin-β-diglucoside, zeaxanthin, and canthaxanthin.

28. A methylotrophic microorganism comprising at least one foreign nucleic acid molecule integrated in the fliC region of the genome.

29. The methylotrophic microorganism of claim 28 wherein the methylotrophic microorganism is a methylotrophic bacteria.

30. The methylotrophic bacteria of claim 29 wherein the methylotrophic bacteria is selected from the group consisting of Methylomonas, Methylobacter, Methylococcus, Methylosinus, Methylocyctis, Methylomicrobium, and Methanomonas.

31. The methylotrophic bacteria according to claim 30 wherein Methylomonas sp. comprises a 16S rRNA gene as represented by SEQ ID NO: 43.

32. An isolated nucleic acid molecule encoding a fliC promoter selected from the group consisting of:
a) an isolated nucleic acid molecule as represented by SEQ ID NO: 34.
b) an isolated nucleic acid molecule that hybridizes with (a) under the following hybridization conditions: 0.1×SSC, 0.1% SDS, 65° C. and washed with 2×SSC, 0.1% SDS followed by 0.1×SSC, 0.1% SDS at 65° C.; and
c) an isolated nucleic acid molecule having at least 95% identity to SEQ ID NO: 34.

33. A method for the expression of a coding region of interest in a recombinant methylotrophic bacteria comprising:
a) providing a recombinant methylotrophic bacteria having a chimeric gene comprising:
i) the isolated nucleic acid molecule of claim 32 encoding a fliC promoter; and
ii) a coding region of interest expressible in a methylotrophic bacteria
wherein the isolated nucleic acid molecule encoding said fliC promoter is operably linked to said coding region of interest; and
b) growing the recombinant methylotrophic bacteria under conditions wherein said chimeric gene is expressed.

34. A method according to claim 33 wherein the coding regions of interest encode at least one carotenoid enzyme selected from the group consisting of geranylgeranyl pyrophosphate synthase, zeaxanthin glucosyl transferase; lycopene cyclase, phytoene desaturase, phytoene synthase, β-carotene hydroxylase, β-carotene ketolase and isopentenyl diphosphate isomerase.

35. A method according to claim 34 wherein said coding region of interest is selected for the group consisting of crtE, crtY, crtI, crtB, crtW, crtZ, and idi.

36. A method according to claim 35 where said coding region of interest is a gene cluster comprising crtE, crtY, crI, crtB, crtW, crtZ, and idi.

37. A method according to claim 36 wherein said gene cluster is selected from the group consisting of SEQ ID NO: 5, SEQ ID NO: 6, SEQ ID NO: 7, and SEQ ID NO: 8.


SEQUENCE LISTING (43 sequences)

SEQ ID NO: 1 - DNA, 12192 bp, artificial sequence
  Plasmid pDCQ334
accttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgacgtctaagaaaccattattatcatgacattaacctataaaaataggcgtatcacgaggccctttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattcactagtaaggaggaataaaccatgagcgcccatgccctgccgaaagccgacctgaccgcgaccagcctgatcgtcagcggtggcatcatcgcggcctggctggcgctgcatgtccatgccctgtggttcctggacgccgccgcccatccgatcctggccatcgccaacttcctgggcctgacctggctgagcgtcggcctgttcatcatcgcgcatgacgccatgcatggcagcgtggtcccgggtcgtccgcgtgccaacgccgccatgggccaactggtcctgtggttgtatgccggcttcagctggcgcaagatgatcgtcaaacatatggcccatcatcgccacgcgggcaccgacgacgatccggacttcgaccatggtggcccggtccgctggtatgcgcgcttcatcggcacctatttcggctggcgtgaaggcctgttgctgccggtcatcgtcaccgtctatgcgctgatcctgggcgaccgctggatgtatgtcgtcttctggccgctgccgagcatcctggcgagcatccaactgttcgtcttcggtacctggctgccgcatcgcccgggccatgacgcctttccggaccgccataacgcccgcagcagccgcatcagcgacccggtcagcctgctgacctgcttccatttcggcggctatcatcatgaacatcatctgcatccgaccgtcccgtggtggcgcctgccgagcacccgcaccaaaggcgacaccgcgtgatctagaaaggaggaataaaccatgaccaacttcctgatcgtcgtcgccaccgtgctggtcatggaactgaccgcgtatagcgtccatcgttggatcatgcatggtccgttgggttggggctggcacaagagccatcatgaagaacatgaccatgccttggaaaagaatgacctgtatggcttggtcttcgccgtcatcgccaccgtcctgttcaccgtcggctggatctgggctccagtcttgtggtggatcgccttgggcatgaccgtctatggcttgatctacttcgtcctgcatgatggcttggtccatcaacgctggccgttccgctacatcccgcgcaaaggctatgcccgtcgcttgtatcaagcccatcgcttgcatcatgccgtcgaaggtcgtgatcattgcgtcagcttcggcttcatctatgccccaccggtcgacaaactgaaacaagacctgaagatgagcggcgtcttgcgtgccgaagcccaagaacgcacctaatgtacagctagtcgagacgccgggtaccaaccatgacaagaccctttgaaacacatcccggtcacgacggggaactgcatgagctgcacgctgccctgcaacgtcgcctggatgaactgctgcccgttggcgatgagcgggatcgggtcagcagcgcaatgcgcgaaggcgtactggcaccggggaaacgcattcgcccgctgctcctgatcctcgccgcccgcgacctcggctgcgatcgcgaccaccccggcctgctggatatggcctgtgcggtggaaatggtgcacgcctcgtcgctgatcctcgacgatattccctgcatggataacgcggcgctccggcgcggtcgccctaccattcatcgccagtatggtgaagacgtggcaattctcgctgcggtagcgttgctcagcagcgcctttggcgtgatggtcgcggcgcagggattgtctcccgagtgccgcagccaggcggtggcggagctgtcgatggcggtcggtacccagggtctggtgcagggtcagtataaggatctgcgtgaaggcaccgccccgcgcagcgccgaggagatcgccaccaccaacgaactgaaaaccagcgtgctgtttggtgccacgctgcaaatcgcggccctggcggcaggcgcctcgccggcggcgcgccagaaaatgcgctgctttgcgcaggatttaggccaggcgttccagctgctggacgatctggcggacggccatgccgggaccggcaaagacatcaataaggacgcgggtaagtccacgctggtggcgatgctcggcagcgacgcggtgcgcgagcggctcgacacccatctgcgccgcgcagacgcccatttttcacgcgcctgcggaaaaaaccaggccacgcgacgctttatgcacgcctggttttcaaaacagctggccgcgtttagctgagcaacggatacaccccggtaatatttgtggagatcacatgaaggacgcgcatctggttcagcgtaaaaatgaccacctggatatcgtgctgcaccctgaccgggcgatgagtaccattcgcaccggatttgacgcctggcgttttgaacactgcgccctcccggagctggatctcgacggtatcgatctctccaccaccctgttttcccgcccgctgaaagccccggtgctgatcagctccatgaccggcggcgcggcgcgcgccagagacattaaccgtcatctggcccaggcggcgcaaacccttgggctggcgatgggcgtcggttcccagcgcgtggcgctggaggacggcgcgcagcacgggctggatgcccagctacgccatatcgccccggacgtgccgctgctggctaaccttggcgcggcgcagatccgcggtgcgcaggggctggactacgcccggcgcgcggtggacatgatcgacgccgacgcgttaattgtgcatctgaacccgctgcaggaggcgctccagggcggcggcgatcgcgactggcgcggcatcctcaacgccattgcgcagctggtgcgcgacctgccggtaccggtggtggttaaagaggtgggcgccgggatctccccggacgttgcctgccgactggcggacgtcggcgtggcgatgatcgacattgccggcgcgggcggaaccagctgggcggcggtggaagctgaacgcgccccgacccccgaggcgcgaaatgtggcgatggcctttgccgactggggcattcctactgccgatgcgctgcgtcgcgtccatcttgcgctgcctgatatcccgcttatcgcctccggcggcatcgccaacggcattgacgcagcaaaagccatcgcgctgggtgcagatctggtgggccaggccgcggcggtgctggcgcatgccaacgcctccggcgacgcggcaattgcccatttccgcaccctgattacgcagctgcggatcgcctgtttctgtaccggcagtgcaaacctgcaggcgttgcgacacgccacgctgcttccggtcaacggcggcgcatccctgtgacgcagtacggtgccttataccggggagcggtatgaaaaaatgggatctgattctggtcggcgcggggctggccaacgggcttatcgcctggcgactaaagcagcgtcatccgacgcttgctgtattaatgctggagtgcggcgacgcgcccggcggaaaccacacctggtcctttcaccaacacgatatcacgccagcccagcacgcctggctggcgccgctggtggcccatcgctgggacgggtacgacgtccactttccgaacgtgtcgcgcaccctgcatgacggctacctgaccatcacctccacgcgttttgcccaagcgatgcgcgggctgatgaaagagaatttgctgacaaacgtgaccgtgtcacgggtgagcgggcaggaagtaaccctcagcgacggacgacgctttaccgccggggcggtgattgatggccgcggctatcagccctcgccgcacctcagcattggctatcaggcgttcatcggccaggagtggcaactgaccgcgccccacgggttaacgcgcccgatcctgatggatgcccgcgtcgcccagggcaacggctaccgctttgtctataccctgccgctcagcgccgacaccctgcttatcgaagacacgcactacattgacggcccgacgctcgacgccgattcagcccgcgcgcggattgccgattacgcccgccagcagggctggcagcttgcgcggctggtgcgtgaggaacagggggcgctgccgatcaccctgtccggcgatccggccgccttctggcaccagttccatcatcagccggtcagcggcctgcgcgccggtctgttccatgccaccaccggctattcgctgccgctggcggttcggctggcggaccgcattgccaacgcgccgggactgcatcagggcgcgctctatcagctgatcgccgatttcgcggcgcgccactggcagacacaacgctttttccgcctgcttaaccgcatgcttttcctggccggcacacccgaccagcgctggcgcgtgatgcagcggttttaccagcttgacgagcagctgatcgcccgtttttatgccggccagcttcgctccgccgaccgcgcgcgcctgctgcttggcaaaccgccggtgccgattgtcggggcgatcaaagccctgctccacactcattcttctctgcgagcccatcataaatgaaacaaaccattgtaattggcgccgggttcggcggactggcgctggcgattcgcctccaggcggcgggcattcctaccacgctgctggagagccgcgacaaacccggcggccgcgcctatgtctacgaagatcgcggctttacctttgatgcgggtcccaccgtcatcaccgatccctccgccattgaggagctgttcaccctcgccggaaaacggctgaaggactacgttgagctgatgccggtgacgccgttctatcgcctgtgctgggaagacggcaaggttttcgactacgccaacgatcaggcggcgcttgagtcgcagatcgccgcgtttaacccgaacgacgtggcgggctatcaccgcttcctcgactactcccgggcggtgtttgccgaaggctatctgaagctcggcgcggtgccgtttctctcgtttcgcgacatgctgcgcgccggtcctcaactggcgcggctgcaggcatggcgcagcgtgtacgacaaagtgtcggcctacgtggaagacgagcacctgcggcaggcattttcgtttcactcgctgctggtgggcggcaacccgttctccacgtcttctatttacaccctgatccacgccctggagcgggaatggggcgtctggttcccgcgcggcggcaccggtgcgctggttcagggcatggtgaagctgtttcaggatcttggcggcaccctcacccttaacgctcaggttgagcggctggagacggtggacaatcaggtgaaggccgtgcatctggttaacgggcagcggctggaggctgcggcggtggcctcgaacgcggacgtggtaaatacctatgcccgactgctcggccatcacccgcacggcgccgctacggccaaaaagctgaaacgcaagcgcatgagcaactcgctgttcgtgctctattttggcctggatcaccatcacacccagctggcgcaccataccgtctgctttggcccgcgttataaagcgctaatcgatgaaattttcagcgccgacaccctgtcggaagatttttcgctctatctgcatgcgccctgcgtaaccgacccgtcgctggccccgccggggtgcggcagctactatgtgctcgcgccggtgccgcacctcggtaacgccccgctcgactggagcgtggaagggccgcgtctgcgggatcgcatttttgattatctcgaagcgcgctatatgccggggctgcgctcccagctggtgacgcaccgcatgttcacgccggaagattttcgcgatacgctcgatgcctggcaggggtcagcgttttcactggagccgatcctcacccagagcgcctggttccggccgcacaaccgcgacagcgtggttgataacctctacctggtcggcgccggaacgcatcccggcgctggcgtgccgggcgtgatcggatccgccaaggcaacggcccagttaatgttaaaggatttagcgtaatgtcccagccgcttctcgaacacgccagcgccaccatgaccgccggttctaaaagtttcgccaccgcctcaaagctgtttgacaaacgcacccggcgcagcgcgctgatgctctatacctggtgccgctactgcgacgatgttatcgacggacaggtggtgggttttgctgccccgaccgagcagagcgacacgcccgaggcgcgcctgcaacggctgcgtaagatgacgcgccgcgcctacgacggggaaaccatgcaagagccgccgttcgccgcctttcaggaggttgccctcgcccatgccattccgcctactcaggccttcgaccacctggaaggctatgcgatggacgtgcgcaacgagcgctattacagcctcgatgatacgctccgctactgttatcacgtggcgggcgtggtcggcctgatgatggccagggtgatgggagtgcgggacgaagccacgctggatcgcgcctgcgatctgggcattgcctttcagctcaccaatatcgccagggatatcgttgacgatgcgcaggtgggacgctgctacctgccgcagcagtggctggcggaagtcggactcaatgaacagacctgcaccgtgcgggccaaccgtccggcgctggcgcgtctggcagcgcggctggtgaccgaggctgagccctattatcagtcagcgcttgccgggctgggggatctgcccctgcgctccgcctgggcgattgccaccgcgcacggggtgtatcgtgagatcggggtgaaggtgctgatggcgggtgaaaaagcatgggatacccgccagggcacgacgcgcgcggagaagctggcgctggttatttccggcgcgaagcaggcgatggcttcccggaaggcgagctggccgccgcgcgatccgcacctctggcagcgcccgcgctagaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcgggacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggacaccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgag

SEQ ID NO: 2 - DNA, 10600 bp, artificial sequence
  Plasmid pDCQ341
accttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgacgtctaagaaaccattattatcatgacattaacctataaaaataggcgtatcacgaggccctttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattcactagaaaggaggaataaaccatgaccgtcgatcacgacgcacggatcagcctgctgctggccgcagccatcggcgccgcgtggctggcgatccatgtcggggcgatcgtgtggtggcgatggagcccggcgacggcggtgctcgcgatccccgtcgtgctcgtacaggcgtggctgagcaccggcctgttcatcgtcgcgcacgattgcatgcacggatcgttcgtgcccggccggcccgcggtcaaccggaccgtcgggacgctgtgcctcggcgcctatgcgggactgtcctatggccagctccatcccaagcatcatgcgcatcacgatgcgccgggcaccgccgccgaccccgatttccatgccggcgcgccgcgatccgcactgccgtggttcgcgcgcttcttcaccagctattacacgcacggccagatcctccggatcaccgcggcggcggtgctgtacatgctgctcggtgtgtcgctgctcaacatcgtcgtgttctgggcgttgccggcgctgatcgcgctggcgcagctgttcgtcttcggcaccttcctgccgcatcgccacggcgacacgccgttcgcggacgcgcacaatgcccgcagcaacggctggccacggctggcgtcgctggcgacctgcttccacttcggcgcctatcatcacgaacatcacctgagcccgtggacgccctggtggcagttgccgcgcgtcggccagcctgccgccggacaccggtcgttaagcaaagaccggtagactagtaccaaccatggatagccattatgaccacccatgtcgacaccacagcacatcagacaagcgaactccttcagctgcagcaaattttacaggcgcatcttgaacatttactgcctgccggacagcaaagcgatcgcgtgcgtgccgcgatgcgtgccggaacgctggcgcagggcaaacgtattcgtcctttattactgctgctggcagcgcgcgatatgggttgcgagctgacgcaaaatggcgttctcgatctcgcctgtgcagtggaaatggtgcacgcggcatcgctgattctggatgacattccctcgatggataacgcgcagatgcgtcgtggtcgccctaccgtgcatcgcgaatttggtgaaaacgtggcgattctcgccgccatcgcgctgcttagccgcgcatttgaagtgattgccattgcacccggtttgcctgccatacataaatctgaagcgattgctgaactctccgctgccgtcggcctgcagggcttagtgcaagggcaattccaggatctgcacgacggcacgcagagccgcagcccggaagcgatcgccatgaccaacgaactgaaaaccagcgtgctgtttcgcgccacgctgcaaatggcggcgattgccgctgacgcttcaccgcaggtgcggcaaagacttagcttcttcgcccaggatttgggccaggcgtttcaactgctcgacgacctcgccgacggttgcaaacacaccggtaaagatgtgcaccaggatcagggcaaatccacgctggtacagatgctcggtgctgacggcgcggaacgtcgcctgcgcgatcacctgcgcagcgcagatgcacaccttgcctgcgcctgccatcgcggcatcgccactcgccaatatatgcacgcgctgtttaatcaacagctagcgatattcaactgaaagtcgtgctggcggaggcgacctgatgcgcacgcaatacgatgtgattttggtcggtgctggactggcgaatggcttgattgcgctgcgtctgcgtcaattgcagccacaactgaaatgcctgttgctggagagcgatgcgcatccggcaggcaatcatacctggtcgtttcatcacagcgatctcagcgccgaacaacttcgctggctgcaaccgctgattaccgtgcgttggtcaggttatcaggtgcgttttcctgcgctgcgccgcaatctggacggggattattgttccatcgcatcaggcgattttgcccgccatctttacgcggcgatgggtgacgatctgtggacaaacacagccgtacaacaggtaaaacccacgcaggtgacgctggcggatggccgtgaacttgctgcgcaagtggtgattgatggtcgcggcctgcagccgacgccacatctgcagctgggttatcaggtgtttcttggacaagagtggcagctggcgcagccgcacggcctgcagcagccgatcctgatggatgccaccgtcgatcagcaagcgggttatcgttttgtctacacgctgccgctcagcgccgatcggctattgattgaagatacccattacgttaaccagcccgcgctggcggagaacaccgctcgtcagcacatcgccgactatgccaatcagcaaggctggacgctgagtacgctgctgcgtgaagagcacggcatattaccgattaccctgagcggcaacatcgatcgattctggcaacagcagcgcggccaagcgtgcagcggcctgcgcgccgggctgtttcatgccaccaccggttactccttgccgtccgccgtggcgctagcggagttggtagcagcgctgttgcccaccgatgccctcacgctcagccaacatatcgaacgctttgcccgtcagcagtggcgcgaacagcgatttttccgtctgctaaaccgcatgctgtttttggccggtaagccgcagcagcgctggcgcgtgatgcaacgtttttaccggctcgatgccgggttaattagccgcttttacgccgggcaactgcgcctgcgcgataaaacgcggattctgtgcggcaagccgccggtgcccatcggtgaagcgctgcgcgcgctgttgaactctgtcgaaccagggaagaaaaaatgaaacgcacttatgtgattggcgcaggctttggcggcctggcgctggcgattcgcctgcaagcggcgggcataccaaccaccttactcgagcagcgcgacaaaccgggcggacgcgcctatgtgtttgaggacagtggctttaccttcgatgccggacccacggtgatcaccgatcccagcgccatcgaagagttgttcacgctggcaggaaaatcgctcagcgattacgtcgagctgatgccggtaacgcccttctatcgcctgtgctgggaagatggcaaacagcttgattacgacaataatcagccgctgctggagcagcagatcgccacgttcaatccgcaagatgtagaaggctatcgtcaatttcttgcctattcacgtgaagtatttagagagggttatctgaaactcggcacggtgccgtttctgcaggtgcgtgacatgctgcgcgtcgcgccgcagttgggacgtctgcaagcatggcgcagcgtctacagcatggtggcgaaatttattcaggacgatcatctgcgtcaggcgttttccttccactcattgctggtgggcggtaatccttttgcaacgtcatcgatctataccttaattcatgcgctggagcgtgaatggggcgtgtggtttccgcgcggcggcaccggcgcgctggtgcagggcatggcgcgactgttcgaggacttgggcggcgagctgttactgaatgccgaagtgagccagctggaaaccagcggcaatcgcattagcggcgttcagttagagggcggacgacgcttcgatgccgccgctgtggcctccaatgccgacgtggtgcatacctacgacaaactgcttcgccaccatccgctggcaatgaaacgtgcgacatcgctgaagcgtaagcgcatgagcaactcgctgtttgtactctattttggcctgaatcagccgcatgaacagctcgcgcaccacaccgtctgttttggcccgcgttatcgtgagttgatcgatgagattttcaacagcagccagctggcagacgatttttcactttacctgcacgcgccctgcagcagcgatccgtcgctggcaccgcccggctgcggcagcttttatgtgttagcgccggtgccgcatctcggcaccgctgacatcgactggcaacaggaaggaccgcgcttgcgcgatcgaatttttgcttatctggagcagcactacatgccgggattacgtcagcaattagtgacacacagaatgtttacgccgtttgattttcgcgacacgctgcatgcccatcacggctcggcgttttcgctggagccgattttgacgcaaagcgcctggttccgcccgcataaccgcgatgccgatatcagcaatctctatctggtgggtgccggtacgcatccaggcgcgggcgtgcccggcgtgatcggttcggccaaggccaccgccaggctgatgctggaggatcgcgccgaatgaatcgacagcctttacttgagcaagtaacgcaaaccatggcggtgggctcgaagagtttcgccaccgccgccaagctgtttgatgcaccgacgcgccgcagcacgctgatgctgtatgcgtggtgtcgtcactgcgatgatgtgattgatgggcaaacgctgggcgaaggcggcacgcagcatgccgtcgaagacgcgcaggcacgtatgcagcatctgcaaattgaaacccgccgcgcctacagcggcgcgcacatggatgaaccggcgtttagggcgtttcaggaagtggcgatcattcaccagctgccgcaacaactggcgtttgatcatctggaaggcttcgctatggatgcacgcaacgaacattacgcgagcttcgatgacacgctgcgttactgctatcacgtcgcgggcgtggtcggtttgatgatggcgcgcgtaatgggcgtgcgcgacgaagcggtgctcgatcacgcctgcgatttaggactggcgttccagctcactaacattgcgcgcgacattgtagaagatgccgaaaatggtcgctgctatctgccgcaatcctggctcgatcaggcgggattacgcgccgatacgctgactgcaccgcaacatcgtgcagcgctcgcctcactggcagcgcgtttagtggcggaggcggaaccctattatcactcggcgcgatccggtttaccgggtttaccgctgcgctcggcgtgggccatcgctacggctcgcggcgtttatcgcgaaattggcgtcaaagttcagcacgccggtgtgcacgcctgggattcacggcagcgcaccagtaaaggtgaaaaactggcgctgctggtgaaaggggcaggtttggcgatcacttcgcgtgtgtctcgtcctgaaccgcgtccggctggtctgtggcagcgtcctcgttgaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcgggacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggacaccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgag

SEQ ID NO: 3 - DNA, 11107 bp, artificial sequence
  Plasmid pDCQ343
accttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgacgtctaagaaaccattattatcatgacattaacctataaaaataggcgtatcacgaggccctttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattcactagaaaggaggaataaaccatgaccgtcgatcacgacgcacggatcagcctgctgctggccgcagccatcggcgccgcgtggctggcgatccatgtcggggcgatcgtgtggtggcgatggagcccggcgacggcggtgctcgcgatccccgtcgtgctcgtacaggcgtggctgagcaccggcctgttcatcgtcgcgcacgattgcatgcacggatcgttcgtgcccggccggcccgcggtcaaccggaccgtcgggacgctgtgcctcggcgcctatgcgggactgtcctatggccagctccatcccaagcatcatgcgcatcacgatgcgccgggcaccgccgccgaccccgatttccatgccggcgcgccgcgatccgcactgccgtggttcgcgcgcttcttcaccagctattacacgcacggccagatcctccggatcaccgcggcggcggtgctgtacatgctgctcggtgtgtcgctgctcaacatcgtcgtgttctgggcgttgccggcgctgatcgcgctggcgcagctgttcgtcttcggcaccttcctgccgcatcgccacggcgacacgccgttcgcggacgcgcacaatgcccgcagcaacggctggccacggctggcgtcgctggcgacctgcttccacttcggcgcctatcatcacgaacatcacctgagcccgtggacgccctggtggcagttgccgcgcgtcggccagcctgccgccggacaccggtcgttaagcaaagaccggtagactagaaaggaggaataaaccatgtcctggccgacgatgatcctgctgttcctcgccaccttcctggggatggaggtcttcgcctgggcgatgcatcgctatgtcatgcacggcctgctgtggacctggcaccgcagccatcatgagccgcacgacgacgtgctggaaaggaacgacctgttcgcggtggtgttcgccgccccggccatcatcctcgtcgccttgggtctacatctgtggccttggatgctgccgatcggcctgggcgttacggcctatggactggtttatttcttctttcacgacgggctggtgcatcgccggttcccgacagggatcgcagggcgctcggcgttctggacgcgacgcattcaggcccaccggctgcatcacgcggtgcggacacgcgagggctgcgtatcgttcggcttcctttgggtgcggtcggcgcgcgcgctgaaggccgaactgtctcagaaacgcggctcatccagcaacggcgcctgaactagtaccaaccatggatagccattatgaccacccatgtcgacaccacagcacatcagacaagcgaactccttcagctgcagcaaattttacaggcgcatcttgaacatttactgcctgccggacagcaaagcgatcgcgtgcgtgccgcgatgcgtgccggaacgctggcgcagggcaaacgtattcgtcctttattactgctgctggcagcgcgcgatatgggttgcgagctgacgcaaaatggcgttctcgatctcgcctgtgcagtggaaatggtgcacgcggcatcgctgattctggatgacattccctcgatggataacgcgcagatgcgtcgtggtcgccctaccgtgcatcgcgaatttggtgaaaacgtggcgattctcgccgccatcgcgctgcttagccgcgcatttgaagtgattgccattgcacccggtttgcctgccatacataaatctgaagcgattgctgaactctccgctgccgtcggcctgcagggcttagtgcaagggcaattccaggatctgcacgacggcacgcagagccgcagcccggaagcgatcgccatgaccaacgaactgaaaaccagcgtgctgtttcgcgccacgctgcaaatggcggcgattgccgctgacgcttcaccgcaggtgcggcaaagacttagcttcttcgcccaggatttgggccaggcgtttcaactgctcgacgacctcgccgacggttgcaaacacaccggtaaagatgtgcaccaggatcagggcaaatccacgctggtacagatgctcggtgctgacggcgcggaacgtcgcctgcgcgatcacctgcgcagcgcagatgcacaccttgcctgcgcctgccatcgcggcatcgccactcgccaatatatgcacgcgctgtttaatcaacagctagcgatattcaactgaaagtcgtgctggcggaggcgacctgatgcgcacgcaatacgatgtgattttggtcggtgctggactggcgaatggcttgattgcgctgcgtctgcgtcaattgcagccacaactgaaatgcctgttgctggagagcgatgcgcatccggcaggcaatcatacctggtcgtttcatcacagcgatctcagcgccgaacaacttcgctggctgcaaccgctgattaccgtgcgttggtcaggttatcaggtgcgttttcctgcgctgcgccgcaatctggacggggattattgttccatcgcatcaggcgattttgcccgccatctttacgcggcgatgggtgacgatctgtggacaaacacagccgtacaacaggtaaaacccacgcaggtgacgctggcggatggccgtgaacttgctgcgcaagtggtgattgatggtcgcggcctgcagccgacgccacatctgcagctgggttatcaggtgtttcttggacaagagtggcagctggcgcagccgcacggcctgcagcagccgatcctgatggatgccaccgtcgatcagcaagcgggttatcgttttgtctacacgctgccgctcagcgccgatcggctattgattgaagatacccattacgttaaccagcccgcgctggcggagaacaccgctcgtcagcacatcgccgactatgccaatcagcaaggctggacgctgagtacgctgctgcgtgaagagcacggcatattaccgattaccctgagcggcaacatcgatcgattctggcaacagcagcgcggccaagcgtgcagcggcctgcgcgccgggctgtttcatgccaccaccggttactccttgccgtccgccgtggcgctagcggagttggtagcagcgctgttgcccaccgatgccctcacgctcagccaacatatcgaacgctttgcccgtcagcagtggcgcgaacagcgatttttccgtctgctaaaccgcatgctgtttttggccggtaagccgcagcagcgctggcgcgtgatgcaacgtttttaccggctcgatgccgggttaattagccgcttttacgccgggcaactgcgcctgcgcgataaaacgcggattctgtgcggcaagccgccggtgcccatcggtgaagcgctgcgcgcgctgttgaactctgtcgaaccagggaagaaaaaatgaaacgcacttatgtgattggcgcaggctttggcggcctggcgctggcgattcgcctgcaagcggcgggcataccaaccaccttactcgagcagcgcgacaaaccgggcggacgcgcctatgtgtttgaggacagtggctttaccttcgatgccggacccacggtgatcaccgatcccagcgccatcgaagagttgttcacgctggcaggaaaatcgctcagcgattacgtcgagctgatgccggtaacgcccttctatcgcctgtgctgggaagatggcaaacagcttgattacgacaataatcagccgctgctggagcagcagatcgccacgttcaatccgcaagatgtagaaggctatcgtcaatttcttgcctattcacgtgaagtatttagagagggttatctgaaactcggcacggtgccgtttctgcaggtgcgtgacatgctgcgcgtcgcgccgcagttgggacgtctgcaagcatggcgcagcgtctacagcatggtggcgaaatttattcaggacgatcatctgcgtcaggcgttttccttccactcattgctggtgggcggtaatccttttgcaacgtcatcgatctataccttaattcatgcgctggagcgtgaatggggcgtgtggtttccgcgcggcggcaccggcgcgctggtgcagggcatggcgcgactgttcgaggacttgggcggcgagctgttactgaatgccgaagtgagccagctggaaaccagcggcaatcgcattagcggcgttcagttagagggcggacgacgcttcgatgccgccgctgtggcctccaatgccgacgtggtgcatacctacgacaaactgcttcgccaccatccgctggcaatgaaacgtgcgacatcgctgaagcgtaagcgcatgagcaactcgctgtttgtactctattttggcctgaatcagccgcatgaacagctcgcgcaccacaccgtctgttttggcccgcgttatcgtgagttgatcgatgagattttcaacagcagccagctggcagacgatttttcactttacctgcacgcgccctgcagcagcgatccgtcgctggcaccgcccggctgcggcagcttttatgtgttagcgccggtgccgcatctcggcaccgctgacatcgactggcaacaggaaggaccgcgcttgcgcgatcgaatttttgcttatctggagcagcactacatgccgggattacgtcagcaattagtgacacacagaatgtttacgccgtttgattttcgcgacacgctgcatgcccatcacggctcggcgttttcgctggagccgattttgacgcaaagcgcctggttccgcccgcataaccgcgatgccgatatcagcaatctctatctggtgggtgccggtacgcatccaggcgcgggcgtgcccggcgtgatcggttcggccaaggccaccgccaggctgatgctggaggatcgcgccgaatgaatcgacagcctttacttgagcaagtaacgcaaaccatggcggtgggctcgaagagtttcgccaccgccgccaagctgtttgatgcaccgacgcgccgcagcacgctgatgctgtatgcgtggtgtcgtcactgcgatgatgtgattgatgggcaaacgctgggcgaaggcggcacgcagcatgccgtcgaagacgcgcaggcacgtatgcagcatctgcaaattgaaacccgccgcgcctacagcggcgcgcacatggatgaaccggcgtttagggcgtttcaggaagtggcgatcattcaccagctgccgcaacaactggcgtttgatcatctggaaggcttcgctatggatgcacgcaacgaacattacgcgagcttcgatgacacgctgcgttactgctatcacgtcgcgggcgtggtcggtttgatgatggcgcgcgtaatgggcgtgcgcgacgaagcggtgctcgatcacgcctgcgatttaggactggcgttccagctcactaacattgcgcgcgacattgtagaagatgccgaaaatggtcgctgctatctgccgcaatcctggctcgatcaggcgggattacgcgccgatacgctgactgcaccgcaacatcgtgcagcgctcgcctcactggcagcgcgtttagtggcggaggcggaaccctattatcactcggcgcgatccggtttaccgggtttaccgctgcgctcggcgtgggccatcgctacggctcgcggcgtttatcgcgaaattggcgtcaaagttcagcacgccggtgtgcacgcctgggattcacggcagcgcaccagtaaaggtgaaaaactggcgctgctggtgaaaggggcaggtttggcgatcacttcgcgtgtgtctcgtcctgaaccgcgtccggctggtctgtggcagcgtcctcgttgaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcgggacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggacaccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgag

SEQ ID NO: 4 - DNA, 12234 bp, artificial sequence
  Plasmid pDCQ377
accttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgacgtctaagaaaccattattatcatgacattaacctataaaaataggcgtatcacgaggccctttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattcactagcaaggaggaataaaccatgcggcaagcgaacaggatgcttaccgggccgcgatgcgctaagtgtcgcgccatgtccgccgtcacgccaatgtcacgggtcgtcccgaaccaggccctgatcggcctgacgctggctggcctgatcgccgcggcctggctgaccctgcacatctacggcgtctattttcatcgctggacgatctggagcgtcctgaccgttccgctgatcgtcgccggccagacctggctatccgtcggcctgttcatcgtcgcccacgacgccatgcacggctcgctggccccggcacgcccacggctgaacacggcgatcggcagcctggcgctggccctctacgccggatttcggttcacgcctttgaagaccgcacaccacgcccatcacgctgcgcccggtacggcggacgatcccgactttcacgccgacgccccgcgcgctttcctgccctggttctacggctttttccgcacctatttcggctggcgagaactggccgttctgacggtgctcgtggccgttgcggtgctgatcctcggcgcccgtatgcccaatcttctggtcttttgggccgcgcccgccctgctctcggcgctacagcttttcacattcggcacctggctgcctcataggcacaccgacgacgccttccccgacaaccacaacgcccgcaccagccccttcggcccggtcctgtcgttgctcacctgcttccacttcggccgccaccacgaacaccacctgaccccctggaagccctggtggagtttgttcagctagactagaaaggaggaataaaccatgtcctggccgacgatgatcctgctgttcctcgccaccttcctggggatggaggtcttcgcctgggcgatgcatcgctatgtcatgcacggcctgctgtggacctggcaccgcagccatcatgagccgcacgacgacgtgctggaaaggaacgacctgttcgcggtggtgttcgccgccccggccatcatcctcgtcgccttgggtctacatctgtggccttggatgctgccgatcggcctgggcgttacggcctatggactggtttatttcttctttcacgacgggctggtgcatcgccggttcccgacagggatcgcagggcgctcggcgttctggacgcgacgcattcaggcccaccggctgcatcacgcggtgcggacacgcgagggctgcgtatcgttcggcttcctttgggtgcggtcggcgcgcgcgctgaaggccgaactgtctcagaaacgcggctcatccagcaacggcgcctgaactagtcgagacgccgggtaccaaccatgacaagaccctttgaaacacatcccggtcacgacggggaactgcatgagctgcacgctgccctgcaacgtcgcctggatgaactgctgcccgttggcgatgagcgggatcgggtcagcagcgcaatgcgcgaaggcgtactggcaccggggaaacgcattcgcccgctgctcctgatcctcgccgcccgcgacctcggctgcgatcgcgaccaccccggcctgctggatatggcctgtgcggtggaaatggtgcacgcctcgtcgctgatcctcgacgatattccctgcatggataacgcggcgctccggcgcggtcgccctaccattcatcgccagtatggtgaagacgtggcaattctcgctgcggtagcgttgctcagcagcgcctttggcgtgatggtcgcggcgcagggattgtctcccgagtgccgcagccaggcggtggcggagctgtcgatggcggtcggtacccagggtctggtgcagggtcagtataaggatctgcgtgaaggcaccgccccgcgcagcgccgaggagatcgccaccaccaacgaactgaaaaccagcgtgctgtttggtgccacgctgcaaatcgcggccctggcggcaggcgcctcgccggcggcgcgccagaaaatgcgctgctttgcgcaggatttaggccaggcgttccagctgctggacgatctggcggacggccatgccgggaccggcaaagacatcaataaggacgcgggtaagtccacgctggtggcgatgctcggcagcgacgcggtgcgcgagcggctcgacacccatctgcgccgcgcagacgcccatttttcacgcgcctgcggaaaaaaccaggccacgcgacgctttatgcacgcctggttttcaaaacagctggccgcgtttagctgagcaacggatacaccccggtaatatttgtggagatcacatgaaggacgcgcatctggttcagcgtaaaaatgaccacctggatatcgtgctgcaccctgaccgggcgatgagtaccattcgcaccggatttgacgcctggcgttttgaacactgcgccctcccggagctggatctcgacggtatcgatctctccaccaccctgttttcccgcccgctgaaagccccggtgctgatcagctccatgaccggcggcgcggcgcgcgccagagacattaaccgtcatctggcccaggcggcgcaaacccttgggctggcgatgggcgtcggttcccagcgcgtggcgctggaggacggcgcgcagcacgggctggatgcccagctacgccatatcgccccggacgtgccgctgctggctaaccttggcgcggcgcagatccgcggtgcgcaggggctggactacgcccggcgcgcggtggacatgatcgacgccgacgcgttaattgtgcatctgaacccgctgcaggaggcgctccagggcggcggcgatcgcgactggcgcggcatcctcaacgccattgcgcagctggtgcgcgacctgccggtaccggtggtggttaaagaggtgggcgccgggatctccccggacgttgcctgccgactggcggacgtcggcgtggcgatgatcgacattgccggcgcgggcggaaccagctgggcggcggtggaagctgaacgcgccccgacccccgaggcgcgaaatgtggcgatggcctttgccgactggggcattcctactgccgatgcgctgcgtcgcgtccatcttgcgctgcctgatatcccgcttatcgcctccggcggcatcgccaacggcattgacgcagcaaaagccatcgcgctgggtgcagatctggtgggccaggccgcggcggtgctggcgcatgccaacgcctccggcgacgcggcaattgcccatttccgcaccctgattacgcagctgcggatcgcctgtttctgtaccggcagtgcaaacctgcaggcgttgcgacacgccacgctgcttccggtcaacggcggcgcatccctgtgacgcagtacggtgccttataccggggagcggtatgaaaaaatgggatctgattctggtcggcgcggggctggccaacgggcttatcgcctggcgactaaagcagcgtcatccgacgcttgctgtattaatgctggagtgcggcgacgcgcccggcggaaaccacacctggtcctttcaccaacacgatatcacgccagcccagcacgcctggctggcgccgctggtggcccatcgctgggacgggtacgacgtccactttccgaacgtgtcgcgcaccctgcatgacggctacctgaccatcacctccacgcgttttgcccaagcgatgcgcgggctgatgaaagagaatttgctgacaaacgtgaccgtgtcacgggtgagcgggcaggaagtaaccctcagcgacggacgacgctttaccgccggggcggtgattgatggccgcggctatcagccctcgccgcacctcagcattggctatcaggcgttcatcggccaggagtggcaactgaccgcgccccacgggttaacgcgcccgatcctgatggatgcccgcgtcgcccagggcaacggctaccgctttgtctataccctgccgctcagcgccgacaccctgcttatcgaagacacgcactacattgacggcccgacgctcgacgccgattcagcccgcgcgcggattgccgattacgcccgccagcagggctggcagcttgcgcggctggtgcgtgaggaacagggggcgctgccgatcaccctgtccggcgatccggccgccttctggcaccagttccatcatcagccggtcagcggcctgcgcgccggtctgttccatgccaccaccggctattcgctgccgctggcggttcggctggcggaccgcattgccaacgcgccgggactgcatcagggcgcgctctatcagctgatcgccgatttcgcggcgcgccactggcagacacaacgctttttccgcctgcttaaccgcatgcttttcctggccggcacacccgaccagcgctggcgcgtgatgcagcggttttaccagcttgacgagcagctgatcgcccgtttttatgccggccagcttcgctccgccgaccgcgcgcgcctgctgcttggcaaaccgccggtgccgattgtcggggcgatcaaagccctgctccacactcattcttctctgcgagcccatcataaatgaaacaaaccattgtaattggcgccgggttcggcggactggcgctggcgattcgcctccaggcggcgggcattcctaccacgctgctggagagccgcgacaaacccggcggccgcgcctatgtctacgaagatcgcggctttacctttgatgcgggtcccaccgtcatcaccgatccctccgccattgaggagctgttcaccctcgccggaaaacggctgaaggactacgttgagctgatgccggtgacgccgttctatcgcctgtgctgggaagacggcaaggttttcgactacgccaacgatcaggcggcgcttgagtcgcagatcgccgcgtttaacccgaacgacgtggcgggctatcaccgcttcctcgactactcccgggcggtgtttgccgaaggctatctgaagctcggcgcggtgccgtttctctcgtttcgcgacatgctgcgcgccggtcctcaactggcgcggctgcaggcatggcgcagcgtgtacgacaaagtgtcggcctacgtggaagacgagcacctgcggcaggcattttcgtttcactcgctgctggtgggcggcaacccgttctccacgtcttctatttacaccctgatccacgccctggagcgggaatggggcgtctggttcccgcgcggcggcaccggtgcgctggttcagggcatggtgaagctgtttcaggatcttggcggcaccctcacccttaacgctcaggttgagcggctggagacggtggacaatcaggtgaaggccgtgcatctggttaacgggcagcggctggaggctgcggcggtggcctcgaacgcggacgtggtaaatacctatgcccgactgctcggccatcacccgcacggcgccgctacggccaaaaagctgaaacgcaagcgcatgagcaactcgctgttcgtgctctattttggcctggatcaccatcacacccagctggcgcaccataccgtctgctttggcccgcgttataaagcgctaatcgatgaaattttcagcgccgacaccctgtcggaagatttttcgctctatctgcatgcgccctgcgtaaccgacccgtcgctggccccgccggggtgcggcagctactatgtgctcgcgccggtgccgcacctcggtaacgccccgctcgactggagcgtggaagggccgcgtctgcgggatcgcatttttgattatctcgaagcgcgctatatgccggggctgcgctcccagctggtgacgcaccgcatgttcacgccggaagattttcgcgatacgctcgatgcctggcaggggtcagcgttttcactggagccgatcctcacccagagcgcctggttccggccgcacaaccgcgacagcgtggttgataacctctacctggtcggcgccggaacgcatcccggcgctggcgtgccgggcgtgatcggatccgccaaggcaacggcccagttaatgttaaaggatttagcgtaatgtcccagccgcttctcgaacacgccagcgccaccatgaccgccggttctaaaagtttcgccaccgcctcaaagctgtttgacaaacgcacccggcgcagcgcgctgatgctctatacctggtgccgctactgcgacgatgttatcgacggacaggtggtgggttttgctgccccgaccgagcagagcgacacgcccgaggcgcgcctgcaacggctgcgtaagatgacgcgccgcgcctacgacggggaaaccatgcaagagccgccgttcgccgcctttcaggaggttgccctcgcccatgccattccgcctactcaggccttcgaccacctggaaggctatgcgatggacgtgcgcaacgagcgctattacagcctcgatgatacgctccgctactgttatcacgtggcgggcgtggtcggcctgatgatggccagggtgatgggagtgcgggacgaagccacgctggatcgcgcctgcgatctgggcattgcctttcagctcaccaatatcgccagggatatcgttgacgatgcgcaggtgggacgctgctacctgccgcagcagtggctggcggaagtcggactcaatgaacagacctgcaccgtgcgggccaaccgtccggcgctggcgcgtctggcagcgcggctggtgaccgaggctgagccctattatcagtcagcgcttgccgggctgggggatctgcccctgcgctccgcctgggcgattgccaccgcgcacggggtgtatcgtgagatcggggtgaaggtgctgatggcgggtgaaaaagcatgggatacccgccagggcacgacgcgcgcggagaagctggcgctggttatttccggcgcgaagcaggcgatggcttcccggaaggcgagctggccgccgcgcgatccgcacctctggcagcgcccgcgctagaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcgggacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggacaccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgag

SEQ ID NO: 5 - DNA, 6866 bp, artificial sequence
  Carotenoid gene cluster
atgagcgcccatgccctgccgaaagccgacctgaccgcgaccagcctgatcgtcagcggtggcatcatcgcggcctggctggcgctgcatgtccatgccctgtggttcctggacgccgccgcccatccgatcctggccatcgccaacttcctgggcctgacctggctgagcgtcggcctgttcatcatcgcgcatgacgccatgcatggcagcgtggtcccgggtcgtccgcgtgccaacgccgccatgggccaactggtcctgtggttgtatgccggcttcagctggcgcaagatgatcgtcaaacatatggcccatcatcgccacgcgggcaccgacgacgatccggacttcgaccatggtggcccggtccgctggtatgcgcgcttcatcggcacctatttcggctggcgtgaaggcctgttgctgccggtcatcgtcaccgtctatgcgctgatcctgggcgaccgctggatgtatgtcgtcttctggccgctgccgagcatcctggcgagcatccaactgttcgtcttcggtacctggctgccgcatcgcccgggccatgacgcctttccggaccgccataacgcccgcagcagccgcatcagcgacccggtcagcctgctgacctgcttccatttcggcggctatcatcatgaacatcatctgcatccgaccgtcccgtggtggcgcctgccgagcacccgcaccaaaggcgacaccgcgtgatctagaaaggaggaataaaccatgaccaacttcctgatcgtcgtcgccaccgtgctggtcatggaactgaccgcgtatagcgtccatcgttggatcatgcatggtccgttgggttggggctggcacaagagccatcatgaagaacatgaccatgccttggaaaagaatgacctgtatggcttggtcttcgccgtcatcgccaccgtcctgttcaccgtcggctggatctgggctccagtcttgtggtggatcgccttgggcatgaccgtctatggcttgatctacttcgtcctgcatgatggcttggtccatcaacgctggccgttccgctacatcccgcgcaaaggctatgcccgtcgcttgtatcaagcccatcgcttgcatcatgccgtcgaaggtcgtgatcattgcgtcagcttcggcttcatctatgccccaccggtcgacaaactgaaacaagacctgaagatgagcggcgtcttgcgtgccgaagcccaagaacgcacctaatgtacagctagtcgagacgccgggtaccaaccatgacaagaccctttgaaacacatcccggtcacgacggggaactgcatgagctgcacgctgccctgcaacgtcgcctggatgaactgctgcccgttggcgatgagcgggatcgggtcagcagcgcaatgcgcgaaggcgtactggcaccggggaaacgcattcgcccgctgctcctgatcctcgccgcccgcgacctcggctgcgatcgcgaccaccccggcctgctggatatggcctgtgcggtggaaatggtgcacgcctcgtcgctgatcctcgacgatattccctgcatggataacgcggcgctccggcgcggtcgccctaccattcatcgccagtatggtgaagacgtggcaattctcgctgcggtagcgttgctcagcagcgcctttggcgtgatggtcgcggcgcagggattgtctcccgagtgccgcagccaggcggtggcggagctgtcgatggcggtcggtacccagggtctggtgcagggtcagtataaggatctgcgtgaaggcaccgccccgcgcagcgccgaggagatcgccaccaccaacgaactgaaaaccagcgtgctgtttggtgccacgctgcaaatcgcggccctggcggcaggcgcctcgccggcggcgcgccagaaaatgcgctgctttgcgcaggatttaggccaggcgttccagctgctggacgatctggcggacggccatgccgggaccggcaaagacatcaataaggacgcgggtaagtccacgctggtggcgatgctcggcagcgacgcggtgcgcgagcggctcgacacccatctgcgccgcgcagacgcccatttttcacgcgcctgcggaaaaaaccaggccacgcgacgctttatgcacgcctggttttcaaaacagctggccgcgtttagctgagcaacggatacaccccggtaatatttgtggagatcacatgaaggacgcgcatctggttcagcgtaaaaatgaccacctggatatcgtgctgcaccctgaccgggcgatgagtaccattcgcaccggatttgacgcctggcgttttgaacactgcgccctcccggagctggatctcgacggtatcgatctctccaccaccctgttttcccgcccgctgaaagccccggtgctgatcagctccatgaccggcggcgcggcgcgcgccagagacattaaccgtcatctggcccaggcggcgcaaacccttgggctggcgatgggcgtcggttcccagcgcgtggcgctggaggacggcgcgcagcacgggctggatgcccagctacgccatatcgccccggacgtgccgctgctggctaaccttggcgcggcgcagatccgcggtgcgcaggggctggactacgcccggcgcgcggtggacatgatcgacgccgacgcgttaattgtgcatctgaacccgctgcaggaggcgctccagggcggcggcgatcgcgactggcgcggcatcctcaacgccattgcgcagctggtgcgcgacctgccggtaccggtggtggttaaagaggtgggcgccgggatctccccggacgttgcctgccgactggcggacgtcggcgtggcgatgatcgacattgccggcgcgggcggaaccagctgggcggcggtggaagctgaacgcgccccgacccccgaggcgcgaaatgtggcgatggcctttgccgactggggcattcctactgccgatgcgctgcgtcgcgtccatcttgcgctgcctgatatcccgcttatcgcctccggcggcatcgccaacggcattgacgcagcaaaagccatcgcgctgggtgcagatctggtgggccaggccgcggcggtgctggcgcatgccaacgcctccggcgacgcggcaattgcccatttccgcaccctgattacgcagctgcggatcgcctgtttctgtaccggcagtgcaaacctgcaggcgttgcgacacgccacgctgcttccggtcaacggcggcgcatccctgtgacgcagtacggtgccttataccggggagcggtatgaaaaaatgggatctgattctggtcggcgcggggctggccaacgggcttatcgcctggcgactaaagcagcgtcatccgacgcttgctgtattaatgctggagtgcggcgacgcgcccggcggaaaccacacctggtcctttcaccaacacgatatcacgccagcccagcacgcctggctggcgccgctggtggcccatcgctgggacgggtacgacgtccactttccgaacgtgtcgcgcaccctgcatgacggctacctgaccatcacctccacgcgttttgcccaagcgatgcgcgggctgatgaaagagaatttgctgacaaacgtgaccgtgtcacgggtgagcgggcaggaagtaaccctcagcgacggacgacgctttaccgccggggcggtgattgatggccgcggctatcagccctcgccgcacctcagcattggctatcaggcgttcatcggccaggagtggcaactgaccgcgccccacgggttaacgcgcccgatcctgatggatgcccgcgtcgcccagggcaacggctaccgctttgtctataccctgccgctcagcgccgacaccctgcttatcgaagacacgcactacattgacggcccgacgctcgacgccgattcagcccgcgcgcggattgccgattacgcccgccagcagggctggcagcttgcgcggctggtgcgtgaggaacagggggcgctgccgatcaccctgtccggcgatccggccgccttctggcaccagttccatcatcagccggtcagcggcctgcgcgccggtctgttccatgccaccaccggctattcgctgccgctggcggttcggctggcggaccgcattgccaacgcgccgggactgcatcagggcgcgctctatcagctgatcgccgatttcgcggcgcgccactggcagacacaacgctttttccgcctgcttaaccgcatgcttttcctggccggcacacccgaccagcgctggcgcgtgatgcagcggttttaccagcttgacgagcagctgatcgcccgtttttatgccggccagcttcgctccgccgaccgcgcgcgcctgctgcttggcaaaccgccggtgccgattgtcggggcgatcaaagccctgctccacactcattcttctctgcgagcccatcataaatgaaacaaaccattgtaattggcgccgggttcggcggactggcgctggcgattcgcctccaggcggcgggcattcctaccacgctgctggagagccgcgacaaacccggcggccgcgcctatgtctacgaagatcgcggctttacctttgatgcgggtcccaccgtcatcaccgatccctccgccattgaggagctgttcaccctcgccggaaaacggctgaaggactacgttgagctgatgccggtgacgccgttctatcgcctgtgctgggaagacggcaaggttttcgactacgccaacgatcaggcggcgcttgagtcgcagatcgccgcgtttaacccgaacgacgtggcgggctatcaccgcttcctcgactactcccgggcggtgtttgccgaaggctatctgaagctcggcgcggtgccgtttctctcgtttcgcgacatgctgcgcgccggtcctcaactggcgcggctgcaggcatggcgcagcgtgtacgacaaagtgtcggcctacgtggaagacgagcacctgcggcaggcattttcgtttcactcgctgctggtgggcggcaacccgttctccacgtcttctatttacaccctgatccacgccctggagcgggaatggggcgtctggttcccgcgcggcggcaccggtgcgctggttcagggcatggtgaagctgtttcaggatcttggcggcaccctcacccttaacgctcaggttgagcggctggagacggtggacaatcaggtgaaggccgtgcatctggttaacgggcagcggctggaggctgcggcggtggcctcgaacgcggacgtggtaaatacctatgcccgactgctcggccatcacccgcacggcgccgctacggccaaaaagctgaaacgcaagcgcatgagcaactcgctgttcgtgctctattttggcctggatcaccatcacacccagctggcgcaccataccgtctgctttggcccgcgttataaagcgctaatcgatgaaattttcagcgccgacaccctgtcggaagatttttcgctctatctgcatgcgccctgcgtaaccgacccgtcgctggccccgccggggtgcggcagctactatgtgctcgcgccggtgccgcacctcggtaacgccccgctcgactggagcgtggaagggccgcgtctgcgggatcgcatttttgattatctcgaagcgcgctatatgccggggctgcgctcccagctggtgacgcaccgcatgttcacgccggaagattttcgcgatacgctcgatgcctggcaggggtcagcgttttcactggagccgatcctcacccagagcgcctggttccggccgcacaaccgcgacagcgtggttgataacctctacctggtcggcgccggaacgcatcccggcgctggcgtgccgggcgtgatcggatccgccaaggcaacggcccagttaatgttaaaggatttagcgtaatgtcccagccgcttctcgaacacgccagcgccaccatgaccgccggttctaaaagtttcgccaccgcctcaaagctgtttgacaaacgcacccggcgcagcgcgctgatgctctatacctggtgccgctactgcgacgatgttatcgacggacaggtggtgggttttgctgccccgaccgagcagagcgacacgcccgaggcgcgcctgcaacggctgcgtaagatgacgcgccgcgcctacgacggggaaaccatgcaagagccgccgttcgccgcctttcaggaggttgccctcgcccatgccattccgcctactcaggccttcgaccacctggaaggctatgcgatggacgtgcgcaacgagcgctattacagcctcgatgatacgctccgctactgttatcacgtggcgggcgtggtcggcctgatgatggccagggtgatgggagtgcgggacgaagccacgctggatcgcgcctgcgatctgggcattgcctttcagctcaccaatatcgccagggatatcgttgacgatgcgcaggtgggacgctgctacctgccgcagcagtggctggcggaagtcggactcaatgaacagacctgcaccgtgcgggccaaccgtccggcgctggcgcgtctggcagcgcggctggtgaccgaggctgagccctattatcagtcagcgcttgccgggctgggggatctgcccctgcgctccgcctgggcgattgccaccgcgcacggggtgtatcgtgagatcggggtgaaggtgctgatggcgggtgaaaaagcatgggatacccgccagggcacgacgcgcgcggagaagctggcgctggttatttccggcgcgaagcaggcgatggcttcccggaaggcgagctggccgccgcgcgatccgcacctctggcagcgcccgcgctag

SEQ ID NO: 6 - DNA, 5275 bp, artificial sequence
  Carotenoid gene cluster
atgaccgtcgatcacgacgcacggatcagcctgctgctggccgcagccatcggcgccgcgtggctggcgatccatgtcggggcgatcgtgtggtggcgatggagcccggcgacggcggtgctcgcgatccccgtcgtgctcgtacaggcgtggctgagcaccggcctgttcatcgtcgcgcacgattgcatgcacggatcgttcgtgcccggccggcccgcggtcaaccggaccgtcgggacgctgtgcctcggcgcctatgcgggactgtcctatggccagctccatcccaagcatcatgcgcatcacgatgcgccgggcaccgccgccgaccccgatttccatgccggcgcgccgcgatccgcactgccgtggttcgcgcgcttcttcaccagctattacacgcacggccagatcctccggatcaccgcggcggcggtgctgtacatgctgctcggtgtgtcgctgctcaacatcgtcgtgttctgggcgttgccggcgctgatcgcgctggcgcagctgttcgtcttcggcaccttcctgccgcatcgccacggcgacacgccgttcgcggacgcgcacaatgcccgcagcaacggctggccacggctggcgtcgctggcgacctgcttccacttcggcgcctatcatcacgaacatcacctgagcccgtggacgccctggtggcagttgccgcgcgtcggccagcctgccgccggacaccggtcgttaagcaaagaccggtagactagtaccaaccatggatagccattatgaccacccatgtcgacaccacagcacatcagacaagcgaactccttcagctgcagcaaattttacaggcgcatcttgaacatttactgcctgccggacagcaaagcgatcgcgtgcgtgccgcgatgcgtgccggaacgctggcgcagggcaaacgtattcgtcctttattactgctgctggcagcgcgcgatatgggttgcgagctgacgcaaaatggcgttctcgatctcgcctgtgcagtggaaatggtgcacgcggcatcgctgattctggatgacattccctcgatggataacgcgcagatgcgtcgtggtcgccctaccgtgcatcgcgaatttggtgaaaacgtggcgattctcgccgccatcgcgctgcttagccgcgcatttgaagtgattgccattgcacccggtttgcctgccatacataaatctgaagcgattgctgaactctccgctgccgtcggcctgcagggcttagtgcaagggcaattccaggatctgcacgacggcacgcagagccgcagcccggaagcgatcgccatgaccaacgaactgaaaaccagcgtgctgtttcgcgccacgctgcaaatggcggcgattgccgctgacgcttcaccgcaggtgcggcaaagacttagcttcttcgcccaggatttgggccaggcgtttcaactgctcgacgacctcgccgacggttgcaaacacaccggtaaagatgtgcaccaggatcagggcaaatccacgctggtacagatgctcggtgctgacggcgcggaacgtcgcctgcgcgatcacctgcgcagcgcagatgcacaccttgcctgcgcctgccatcgcggcatcgccactcgccaatatatgcacgcgctgtttaatcaacagctagcgatattcaactgaaagtcgtgctggcggaggcgacctgatgcgcacgcaatacgatgtgattttggtcggtgctggactggcgaatggcttgattgcgctgcgtctgcgtcaattgcagccacaactgaaatgcctgttgctggagagcgatgcgcatccggcaggcaatcatacctggtcgtttcatcacagcgatctcagcgccgaacaacttcgctggctgcaaccgctgattaccgtgcgttggtcaggttatcaggtgcgttttcctgcgctgcgccgcaatctggacggggattattgttccatcgcatcaggcgattttgcccgccatctttacgcggcgatgggtgacgatctgtggacaaacacagccgtacaacaggtaaaacccacgcaggtgacgctggcggatggccgtgaacttgctgcgcaagtggtgattgatggtcgcggcctgcagccgacgccacatctgcagctgggttatcaggtgtttcttggacaagagtggcagctggcgcagccgcacggcctgcagcagccgatcctgatggatgccaccgtcgatcagcaagcgggttatcgttttgtctacacgctgccgctcagcgccgatcggctattgattgaagatacccattacgttaaccagcccgcgctggcggagaacaccgctcgtcagcacatcgccgactatgccaatcagcaaggctggacgctgagtacgctgctgcgtgaagagcacggcatattaccgattaccctgagcggcaacatcgatcgattctggcaacagcagcgcggccaagcgtgcagcggcctgcgcgccgggctgtttcatgccaccaccggttactccttgccgtccgccgtggcgctagcggagttggtagcagcgctgttgcccaccgatgccctcacgctcagccaacatatcgaacgctttgcccgtcagcagtggcgcgaacagcgatttttccgtctgctaaaccgcatgctgtttttggccggtaagccgcagcagcgctggcgcgtgatgcaacgtttttaccggctcgatgccgggttaattagccgcttttacgccgggcaactgcgcctgcgcgataaaacgcggattctgtgcggcaagccgccggtgcccatcggtgaagcgctgcgcgcgctgttgaactctgtcgaaccagggaagaaaaaatgaaacgcacttatgtgattggcgcaggctttggcggcctggcgctggcgattcgcctgcaagcggcgggcataccaaccaccttactcgagcagcgcgacaaaccgggcggacgcgcctatgtgtttgaggacagtggctttaccttcgatgccggacccacggtgatcaccgatcccagcgccatcgaagagttgttcacgctggcaggaaaatcgctcagcgattacgtcgagctgatgccggtaacgcccttctatcgcctgtgctgggaagatggcaaacagcttgattacgacaataatcagccgctgctggagcagcagatcgccacgttcaatccgcaagatgtagaaggctatcgtcaatttcttgcctattcacgtgaagtatttagagagggttatctgaaactcggcacggtgccgtttctgcaggtgcgtgacatgctgcgcgtcgcgccgcagttgggacgtctgcaagcatggcgcagcgtctacagcatggtggcgaaatttattcaggacgatcatctgcgtcaggcgttttccttccactcattgctggtgggcggtaatccttttgcaacgtcatcgatctataccttaattcatgcgctggagcgtgaatggggcgtgtggtttccgcgcggcggcaccggcgcgctggtgcagggcatggcgcgactgttcgaggacttgggcggcgagctgttactgaatgccgaagtgagccagctggaaaccagcggcaatcgcattagcggcgttcagttagagggcggacgacgcttcgatgccgccgctgtggcctccaatgccgacgtggtgcatacctacgacaaactgcttcgccaccatccgctggcaatgaaacgtgcgacatcgctgaagcgtaagcgcatgagcaactcgctgtttgtactctattttggcctgaatcagccgcatgaacagctcgcgcaccacaccgtctgttttggcccgcgttatcgtgagttgatcgatgagattttcaacagcagccagctggcagacgatttttcactttacctgcacgcgccctgcagcagcgatccgtcgctggcaccgcccggctgcggcagcttttatgtgttagcgccggtgccgcatctcggcaccgctgacatcgactggcaacaggaaggaccgcgcttgcgcgatcgaatttttgcttatctggagcagcactacatgccgggattacgtcagcaattagtgacacacagaatgtttacgccgtttgattttcgcgacacgctgcatgcccatcacggctcggcgttttcgctggagccgattttgacgcaaagcgcctggttccgcccgcataaccgcgatgccgatatcagcaatctctatctggtgggtgccggtacgcatccaggcgcgggcgtgcccggcgtgatcggttcggccaaggccaccgccaggctgatgctggaggatcgcgccgaatgaatcgacagcctttacttgagcaagtaacgcaaaccatggcggtgggctcgaagagtttcgccaccgccgccaagctgtttgatgcaccgacgcgccgcagcacgctgatgctgtatgcgtggtgtcgtcactgcgatgatgtgattgatgggcaaacgctgggcgaaggcggcacgcagcatgccgtcgaagacgcgcaggcacgtatgcagcatctgcaaattgaaacccgccgcgcctacagcggcgcgcacatggatgaaccggcgtttagggcgtttcaggaagtggcgatcattcaccagctgccgcaacaactggcgtttgatcatctggaaggcttcgctatggatgcacgcaacgaacattacgcgagcttcgatgacacgctgcgttactgctatcacgtcgcgggcgtggtcggtttgatgatggcgcgcgtaatgggcgtgcgcgacgaagcggtgctcgatcacgcctgcgatttaggactggcgttccagctcactaacattgcgcgcgacattgtagaagatgccgaaaatggtcgctgctatctgccgcaatcctggctcgatcaggcgggattacgcgccgatacgctgactgcaccgcaacatcgtgcagcgctcgcctcactggcagcgcgtttagtggcggaggcggaaccctattatcactcggcgcgatccggtttaccgggtttaccgctgcgctcggcgtgggccatcgctacggctcgcggcgtttatcgcgaaattggcgtcaaagttcagcacgccggtgtgcacgcctgggattcacggcagcgcaccagtaaaggtgaaaaactggcgctgctggtgaaaggggcaggtttggcgatcacttcgcgtgtgtctcgtcctgaaccgcgtccggctggtctgtggcagcgtcctcgttga

SEQ ID NO: 7 - DNA, 5014 bp, artificial sequence
  Carotenoid gene cluster
atgtcctggccgacgatgatcctgctgttcctcgccaccttcctggggatggaggtcttcgcctgggcgatgcatcgctatgtcatgcacggcctgctgtggacctggcaccgcagccatcatgagccgcacgacgacgtgctggaaaggaacgacctgttcgcggtggtgttcgccgccccggccatcatcctcgtcgccttgggtctacatctgtggccttggatgctgccgatcggcctgggcgttacggcctatggactggtttatttcttctttcacgacgggctggtgcatcgccggttcccgacagggatcgcagggcgctcggcgttctggacgcgacgcattcaggcccaccggctgcatcacgcggtgcggacacgcgagggctgcgtatcgttcggcttcctttgggtgcggtcggcgcgcgcgctgaaggccgaactgtctcagaaacgcggctcatccagcaacggcgcctgaactagtaccaaccatggatagccattatgaccacccatgtcgacaccacagcacatcagacaagcgaactccttcagctgcagcaaattttacaggcgcatcttgaacatttactgcctgccggacagcaaagcgatcgcgtgcgtgccgcgatgcgtgccggaacgctggcgcagggcaaacgtattcgtcctttattactgctgctggcagcgcgcgatatgggttgcgagctgacgcaaaatggcgttctcgatctcgcctgtgcagtggaaatggtgcacgcggcatcgctgattctggatgacattccctcgatggataacgcgcagatgcgtcgtggtcgccctaccgtgcatcgcgaatttggtgaaaacgtggcgattctcgccgccatcgcgctgcttagccgcgcatttgaagtgattgccattgcacccggtttgcctgccatacataaatctgaagcgattgctgaactctccgctgccgtcggcctgcagggcttagtgcaagggcaattccaggatctgcacgacggcacgcagagccgcagcccggaagcgatcgccatgaccaacgaactgaaaaccagcgtgctgtttcgcgccacgctgcaaatggcggcgattgccgctgacgcttcaccgcaggtgcggcaaagacttagcttcttcgcccaggatttgggccaggcgtttcaactgctcgacgacctcgccgacggttgcaaacacaccggtaaagatgtgcaccaggatcagggcaaatccacgctggtacagatgctcggtgctgacggcgcggaacgtcgcctgcgcgatcacctgcgcagcgcagatgcacaccttgcctgcgcctgccatcgcggcatcgccactcgccaatatatgcacgcgctgtttaatcaacagctagcgatattcaactgaaagtcgtgctggcggaggcgacctgatgcgcacgcaatacgatgtgattttggtcggtgctggactggcgaatggcttgattgcgctgcgtctgcgtcaattgcagccacaactgaaatgcctgttgctggagagcgatgcgcatccggcaggcaatcatacctggtcgtttcatcacagcgatctcagcgccgaacaacttcgctggctgcaaccgctgattaccgtgcgttggtcaggttatcaggtgcgttttcctgcgctgcgccgcaatctggacggggattattgttccatcgcatcaggcgattttgcccgccatctttacgcggcgatgggtgacgatctgtggacaaacacagccgtacaacaggtaaaacccacgcaggtgacgctggcggatggccgtgaacttgctgcgcaagtggtgattgatggtcgcggcctgcagccgacgccacatctgcagctgggttatcaggtgtttcttggacaagagtggcagctggcgcagccgcacggcctgcagcagccgatcctgatggatgccaccgtcgatcagcaagcgggttatcgttttgtctacacgctgccgctcagcgccgatcggctattgattgaagatacccattacgttaaccagcccgcgctggcggagaacaccgctcgtcagcacatcgccgactatgccaatcagcaaggctggacgctgagtacgctgctgcgtgaagagcacggcatattaccgattaccctgagcggcaacatcgatcgattctggcaacagcagcgcggccaagcgtgcagcggcctgcgcgccgggctgtttcatgccaccaccggttactccttgccgtccgccgtggcgctagcggagttggtagcagcgctgttgcccaccgatgccctcacgctcagccaacatatcgaacgctttgcccgtcagcagtggcgcgaacagcgatttttccgtctgctaaaccgcatgctgtttttggccggtaagccgcagcagcgctggcgcgtgatgcaacgtttttaccggctcgatgccgggttaattagccgcttttacgccgggcaactgcgcctgcgcgataaaacgcggattctgtgcggcaagccgccggtgcccatcggtgaagcgctgcgcgcgctgttgaactctgtcgaaccagggaagaaaaaatgaaacgcacttatgtgattggcgcaggctttggcggcctggcgctggcgattcgcctgcaagcggcgggcataccaaccaccttactcgagcagcgcgacaaaccgggcggacgcgcctatgtgtttgaggacagtggctttaccttcgatgccggacccacggtgatcaccgatcccagcgccatcgaagagttgttcacgctggcaggaaaatcgctcagcgattacgtcgagctgatgccggtaacgcccttctatcgcctgtgctgggaagatggcaaacagcttgattacgacaataatcagccgctgctggagcagcagatcgccacgttcaatccgcaagatgtagaaggctatcgtcaatttcttgcctattcacgtgaagtatttagagagggttatctgaaactcggcacggtgccgtttctgcaggtgcgtgacatgctgcgcgtcgcgccgcagttgggacgtctgcaagcatggcgcagcgtctacagcatggtggcgaaatttattcaggacgatcatctgcgtcaggcgttttccttccactcattgctggtgggcggtaatccttttgcaacgtcatcgatctataccttaattcatgcgctggagcgtgaatggggcgtgtggtttccgcgcggcggcaccggcgcgctggtgcagggcatggcgcgactgttcgaggacttgggcggcgagctgttactgaatgccgaagtgagccagctggaaaccagcggcaatcgcattagcggcgttcagttagagggcggacgacgcttcgatgccgccgctgtggcctccaatgccgacgtggtgcatacctacgacaaactgcttcgccaccatccgctggcaatgaaacgtgcgacatcgctgaagcgtaagcgcatgagcaactcgctgtttgtactctattttggcctgaatcagccgcatgaacagctcgcgcaccacaccgtctgttttggcccgcgttatcgtgagttgatcgatgagattttcaacagcagccagctggcagacgatttttcactttacctgcacgcgccctgcagcagcgatccgtcgctggcaccgcccggctgcggcagcttttatgtgttagcgccggtgccgcatctcggcaccgctgacatcgactggcaacaggaaggaccgcgcttgcgcgatcgaatttttgcttatctggagcagcactacatgccgggattacgtcagcaattagtgacacacagaatgtttacgccgtttgattttcgcgacacgctgcatgcccatcacggctcggcgttttcgctggagccgattttgacgcaaagcgcctggttccgcccgcataaccgcgatgccgatatcagcaatctctatctggtgggtgccggtacgcatccaggcgcgggcgtgcccggcgtgatcggttcggccaaggccaccgccaggctgatgctggaggatcgcgccgaatgaatcgacagcctttacttgagcaagtaacgcaaaccatggcggtgggctcgaagagtttcgccaccgccgccaagctgtttgatgcaccgacgcgccgcagcacgctgatgctgtatgcgtggtgtcgtcactgcgatgatgtgattgatgggcaaacgctgggcgaaggcggcacgcagcatgccgtcgaagacgcgcaggcacgtatgcagcatctgcaaattgaaacccgccgcgcctacagcggcgcgcacatggatgaaccggcgtttagggcgtttcaggaagtggcgatcattcaccagctgccgcaacaactggcgtttgatcatctggaaggcttcgctatggatgcacgcaacgaacattacgcgagcttcgatgacacgctgcgttactgctatcacgtcgcgggcgtggtcggtttgatgatggcgcgcgtaatgggcgtgcgcgacgaagcggtgctcgatcacgcctgcgatttaggactggcgttccagctcactaacattgcgcgcgacattgtagaagatgccgaaaatggtcgctgctatctgccgcaatcctggctcgatcaggcgggattacgcgccgatacgctgactgcaccgcaacatcgtgcagcgctcgcctcactggcagcgcgtttagtggcggaggcggaaccctattatcactcggcgcgatccggtttaccgggtttaccgctgcgctcggcgtgggccatcgctacggctcgcggcgtttatcgcgaaattggcgtcaaagttcagcacgccggtgtgcacgcctgggattcacggcagcgcaccagtaaaggtgaaaaactggcgctgctggtgaaaggggcaggtttggcgatcacttcgcgtgtgtctcgtcctgaaccgcgtccggctggtctgtggcagcgtcctcgttga

SEQ ID NO: 8 - DNA, 6908 bp, artificial sequence
  Carotenoid gene cluster
atgcggcaagcgaacaggatgcttaccgggccgcgatgcgctaagtgtcgcgccatgtccgccgtcacgccaatgtcacgggtcgtcccgaaccaggccctgatcggcctgacgctggctggcctgatcgccgcggcctggctgaccctgcacatctacggcgtctattttcatcgctggacgatctggagcgtcctgaccgttccgctgatcgtcgccggccagacctggctatccgtcggcctgttcatcgtcgcccacgacgccatgcacggctcgctggccccggcacgcccacggctgaacacggcgatcggcagcctggcgctggccctctacgccggatttcggttcacgcctttgaagaccgcacaccacgcccatcacgctgcgcccggtacggcggacgatcccgactttcacgccgacgccccgcgcgctttcctgccctggttctacggctttttccgcacctatttcggctggcgagaactggccgttctgacggtgctcgtggccgttgcggtgctgatcctcggcgcccgtatgcccaatcttctggtcttttgggccgcgcccgccctgctctcggcgctacagcttttcacattcggcacctggctgcctcataggcacaccgacgacgccttccccgacaaccacaacgcccgcaccagccccttcggcccggtcctgtcgttgctcacctgcttccacttcggccgccaccacgaacaccacctgaccccctggaagccctggtggagtttgttcagctagactagaaaggaggaataaaccatgtcctggccgacgatgatcctgctgttcctcgccaccttcctggggatggaggtcttcgcctgggcgatgcatcgctatgtcatgcacggcctgctgtggacctggcaccgcagccatcatgagccgcacgacgacgtgctggaaaggaacgacctgttcgcggtggtgttcgccgccccggccatcatcctcgtcgccttgggtctacatctgtggccttggatgctgccgatcggcctgggcgttacggcctatggactggtttatttcttctttcacgacgggctggtgcatcgccggttcccgacagggatcgcagggcgctcggcgttctggacgcgacgcattcaggcccaccggctgcatcacgcggtgcggacacgcgagggctgcgtatcgttcggcttcctttgggtgcggtcggcgcgcgcgctgaaggccgaactgtctcagaaacgcggctcatccagcaacggcgcctgaactagtcgagacgccgggtaccaaccatgacaagaccctttgaaacacatcccggtcacgacggggaactgcatgagctgcacgctgccctgcaacgtcgcctggatgaactgctgcccgttggcgatgagcgggatcgggtcagcagcgcaatgcgcgaaggcgtactggcaccggggaaacgcattcgcccgctgctcctgatcctcgccgcccgcgacctcggctgcgatcgcgaccaccccggcctgctggatatggcctgtgcggtggaaatggtgcacgcctcgtcgctgatcctcgacgatattccctgcatggataacgcggcgctccggcgcggtcgccctaccattcatcgccagtatggtgaagacgtggcaattctcgctgcggtagcgttgctcagcagcgcctttggcgtgatggtcgcggcgcagggattgtctcccgagtgccgcagccaggcggtggcggagctgtcgatggcggtcggtacccagggtctggtgcagggtcagtataaggatctgcgtgaaggcaccgccccgcgcagcgccgaggagatcgccaccaccaacgaactgaaaaccagcgtgctgtttggtgccacgctgcaaatcgcggccctggcggcaggcgcctcgccggcggcgcgccagaaaatgcgctgctttgcgcaggatttaggccaggcgttccagctgctggacgatctggcggacggccatgccgggaccggcaaagacatcaataaggacgcgggtaagtccacgctggtggcgatgctcggcagcgacgcggtgcgcgagcggctcgacacccatctgcgccgcgcagacgcccatttttcacgcgcctgcggaaaaaaccaggccacgcgacgctttatgcacgcctggttttcaaaacagctggccgcgtttagctgagcaacggatacaccccggtaatatttgtggagatcacatgaaggacgcgcatctggttcagcgtaaaaatgaccacctggatatcgtgctgcaccctgaccgggcgatgagtaccattcgcaccggatttgacgcctggcgttttgaacactgcgccctcccggagctggatctcgacggtatcgatctctccaccaccctgttttcccgcccgctgaaagccccggtgctgatcagctccatgaccggcggcgcggcgcgcgccagagacattaaccgtcatctggcccaggcggcgcaaacccttgggctggcgatgggcgtcggttcccagcgcgtggcgctggaggacggcgcgcagcacgggctggatgcccagctacgccatatcgccccggacgtgccgctgctggctaaccttggcgcggcgcagatccgcggtgcgcaggggctggactacgcccggcgcgcggtggacatgatcgacgccgacgcgttaattgtgcatctgaacccgctgcaggaggcgctccagggcggcggcgatcgcgactggcgcggcatcctcaacgccattgcgcagctggtgcgcgacctgccggtaccggtggtggttaaagaggtgggcgccgggatctccccggacgttgcctgccgactggcggacgtcggcgtggcgatgatcgacattgccggcgcgggcggaaccagctgggcggcggtggaagctgaacgcgccccgacccccgaggcgcgaaatgtggcgatggcctttgccgactggggcattcctactgccgatgcgctgcgtcgcgtccatcttgcgctgcctgatatcccgcttatcgcctccggcggcatcgccaacggcattgacgcagcaaaagccatcgcgctgggtgcagatctggtgggccaggccgcggcggtgctggcgcatgccaacgcctccggcgacgcggcaattgcccatttccgcaccctgattacgcagctgcggatcgcctgtttctgtaccggcagtgcaaacctgcaggcgttgcgacacgccacgctgcttccggtcaacggcggcgcatccctgtgacgcagtacggtgccttataccggggagcggtatgaaaaaatgggatctgattctggtcggcgcggggctggccaacgggcttatcgcctggcgactaaagcagcgtcatccgacgcttgctgtattaatgctggagtgcggcgacgcgcccggcggaaaccacacctggtcctttcaccaacacgatatcacgccagcccagcacgcctggctggcgccgctggtggcccatcgctgggacgggtacgacgtccactttccgaacgtgtcgcgcaccctgcatgacggctacctgaccatcacctccacgcgttttgcccaagcgatgcgcgggctgatgaaagagaatttgctgacaaacgtgaccgtgtcacgggtgagcgggcaggaagtaaccctcagcgacggacgacgctttaccgccggggcggtgattgatggccgcggctatcagccctcgccgcacctcagcattggctatcaggcgttcatcggccaggagtggcaactgaccgcgccccacgggttaacgcgcccgatcctgatggatgcccgcgtcgcccagggcaacggctaccgctttgtctataccctgccgctcagcgccgacaccctgcttatcgaagacacgcactacattgacggcccgacgctcgacgccgattcagcccgcgcgcggattgccgattacgcccgccagcagggctggcagcttgcgcggctggtgcgtgaggaacagggggcgctgccgatcaccctgtccggcgatccggccgccttctggcaccagttccatcatcagccggtcagcggcctgcgcgccggtctgttccatgccaccaccggctattcgctgccgctggcggttcggctggcggaccgcattgccaacgcgccgggactgcatcagggcgcgctctatcagctgatcgccgatttcgcggcgcgccactggcagacacaacgctttttccgcctgcttaaccgcatgcttttcctggccggcacacccgaccagcgctggcgcgtgatgcagcggttttaccagcttgacgagcagctgatcgcccgtttttatgccggccagcttcgctccgccgaccgcgcgcgcctgctgcttggcaaaccgccggtgccgattgtcggggcgatcaaagccctgctccacactcattcttctctgcgagcccatcataaatgaaacaaaccattgtaattggcgccgggttcggcggactggcgctggcgattcgcctccaggcggcgggcattcctaccacgctgctggagagccgcgacaaacccggcggccgcgcctatgtctacgaagatcgcggctttacctttgatgcgggtcccaccgtcatcaccgatccctccgccattgaggagctgttcaccctcgccggaaaacggctgaaggactacgttgagctgatgccggtgacgccgttctatcgcctgtgctgggaagacggcaaggttttcgactacgccaacgatcaggcggcgcttgagtcgcagatcgccgcgtttaacccgaacgacgtggcgggctatcaccgcttcctcgactactcccgggcggtgtttgccgaaggctatctgaagctcggcgcggtgccgtttctctcgtttcgcgacatgctgcgcgccggtcctcaactggcgcggctgcaggcatggcgcagcgtgtacgacaaagtgtcggcctacgtggaagacgagcacctgcggcaggcattttcgtttcactcgctgctggtgggcggcaacccgttctccacgtcttctatttacaccctgatccacgccctggagcgggaatggggcgtctggttcccgcgcggcggcaccggtgcgctggttcagggcatggtgaagctgtttcaggatcttggcggcaccctcacccttaacgctcaggttgagcggctggagacggtggacaatcaggtgaaggccgtgcatctggttaacgggcagcggctggaggctgcggcggtggcctcgaacgcggacgtggtaaatacctatgcccgactgctcggccatcacccgcacggcgccgctacggccaaaaagctgaaacgcaagcgcatgagcaactcgctgttcgtgctctattttggcctggatcaccatcacacccagctggcgcaccataccgtctgctttggcccgcgttataaagcgctaatcgatgaaattttcagcgccgacaccctgtcggaagatttttcgctctatctgcatgcgccctgcgtaaccgacccgtcgctggccccgccggggtgcggcagctactatgtgctcgcgccggtgccgcacctcggtaacgccccgctcgactggagcgtggaagggccgcgtctgcgggatcgcatttttgattatctcgaagcgcgctatatgccggggctgcgctcccagctggtgacgcaccgcatgttcacgccggaagattttcgcgatacgctcgatgcctggcaggggtcagcgttttcactggagccgatcctcacccagagcgcctggttccggccgcacaaccgcgacagcgtggttgataacctctacctggtcggcgccggaacgcatcccggcgctggcgtgccgggcgtgatcggatccgccaaggcaacggcccagttaatgttaaaggatttagcgtaatgtcccagccgcttctcgaacacgccagcgccaccatgaccgccggttctaaaagtttcgccaccgcctcaaagctgtttgacaaacgcacccggcgcagcgcgctgatgctctatacctggtgccgctactgcgacgatgttatcgacggacaggtggtgggttttgctgccccgaccgagcagagcgacacgcccgaggcgcgcctgcaacggctgcgtaagatgacgcgccgcgcctacgacggggaaaccatgcaagagccgccgttcgccgcctttcaggaggttgccctcgcccatgccattccgcctactcaggccttcgaccacctggaaggctatgcgatggacgtgcgcaacgagcgctattacagcctcgatgatacgctccgctactgttatcacgtggcgggcgtggtcggcctgatgatggccagggtgatgggagtgcgggacgaagccacgctggatcgcgcctgcgatctgggcattgcctttcagctcaccaatatcgccagggatatcgttgacgatgcgcaggtgggacgctgctacctgccgcagcagtggctggcggaagtcggactcaatgaacagacctgcaccgtgcgggccaaccgtccggcgctggcgcgtctggcagcgcggctggtgaccgaggctgagccctattatcagtcagcgcttgccgggctgggggatctgcccctgcgctccgcctgggcgattgccaccgcgcacggggtgtatcgtgagatcggggtgaaggtgctgatggcgggtgaaaaagcatgggatacccgccagggcacgacgcgcgcggagaagctggcgctggttatttccggcgcgaagcaggcgatggcttcccggaaggcgagctggccgccgcgcgatccgcacctctggcagcgcccgcgctag

SEQ ID NO: 9 - DNA, 58 bp, artificial sequence
  Primer
aattcccgggactagtacgcgtgcggccgcccatggcatatgttcgaacccgggtacc

SEQ ID NO: 10 - DNA, 55 bp, artificial sequence
  Primer
ggtacccgggttcgaacatatgccatgggcggccgcacgcgtactagtcccggga

SEQ ID NO: 11 - DNA, 24 bp, artificial sequence
  Primer
gcacgatgaagagcagaagttatc

SEQ ID NO: 12 - DNA, 21 bp, artificial sequence
  Primer
aacacttaacggctgacatgg

SEQ ID NO: 13 - DNA, 38 bp, artificial sequence
  Primer
atgcttcgaacgggtacctaggatgcgtgatctgatcc

SEQ ID NO: 14 - DNA, 26 bp, artificial sequence
  Primer
tggcttcgaacgatgaattgtgtctc

SEQ ID NO: 15 - DNA, 747 bp, Sphingomonas melonis DC18
atgaccgtcgatcacgacgcacggatcagcctgctgctggccgcagccatcggcgccgcgtggctggcgatccatgtcggggcgatcgtgtggtggcgatggagcccggcgacggcggtgctcgcgatccccgtcgtgctcgtacaggcgtggctgagcaccggcctgttcatcgtcgcgcacgattgcatgcacggatcgttcgtgcccggccggcccgcggtcaaccggaccgtcgggacgctgtgcctcggcgcctatgcgggactgtcctatggccagctccatcccaagcatcatgcgcatcacgatgcgccgggcaccgccgccgaccccgatttccatgccggcgcgccgcgatccgcactgccgtggttcgcgcgcttcttcaccagctattacacgcacggccagatcctccggatcaccgcggcggcggtgctgtacatgctgctcggtgtgtcgctgctcaacatcgtcgtgttctgggcgttgccggcgctgatcgcgctggcgcagctgttcgtcttcggcaccttcctgccgcatcgccacggcgacacgccgttcgcggacgcgcacaatgcccgcagcaacggctggccacggctggcgtcgctggcgacctgcttccacttcggcgcctatcatcacgaacatcacctgagcccgtggacgccctggtggcagttgccgcgcgtcggccagcctgccgccggacaccggtcgttaagcaaagaccggtag

SEQ ID NO: 16 - DNA, 486 bp, Brevundimonas vesicularis DC263
atgtcctggccgacgatgatcctgctgttcctcgccaccttcctggggatggaggtcttcgcctgggcgatgcatcgctatgtcatgcacggcctgctgtggacctggcaccgcagccatcatgagccgcacgacgacgtgctggaaaggaacgacctgttcgcggtggtgttcgccgccccggccatcatcctcgtcgccttgggtctacatctgtggccttggatgctgccgatcggcctgggcgttacggcctatggactggtttatttcttctttcacgacgggctggtgcatcgccggttcccgacagggatcgcagggcgctcggcgttctggacgcgacgcattcaggcccaccggctgcatcacgcggtgcggacacgcgagggctgcgtatcgttcggcttcctttgggtgcggtcggcgcgcgcgctgaaggccgaactgtctcagaaacgcggctcatccagcaacggcgcctga

SEQ ID NO: 17 - DNA, 33 bp, artificial sequence
  Primer
tacccactagtaaggaggaataaaccatgaccg

SEQ ID NO: 18 - DNA, 19 bp, artificial sequence
  Primer
ggttggtactagttcaggc

SEQ ID NO: 19 - DNA, 42 bp, artificial sequence
  Primer
atgcttcgaacgggtacctaggcgtttaagggcaccaataac

SEQ ID NO: 20 - DNA, 28 bp, artificial sequence
  Primer
tggcttcgaatacctgtgacggaagatc

SEQ ID NO: 21 - DNA, 20 bp, artificial sequence
  Primer
acatcgtattgcgtgcgcat

SEQ ID NO: 22 - DNA, 24 bp, artificial sequence
  Primer
gaaccacagggcatggacatgcag

SEQ ID NO: 23 - DNA, 25 bp, artificial sequence
  Primer
gcagtttcatttgatgctcgatgag

SEQ ID NO: 24 - DNA, 22 bp, artificial sequence
  Primer
gggcgctcatggtttattcctc

SEQ ID NO: 25 - DNA, 27 bp, artificial sequence
  Primer
gggacggcggctttgttgaataaatcg

SEQ ID NO: 26 - DNA, 19 bp, artificial sequence
  Primer
gacatggatcgccagccac

SEQ ID NO: 27 - DNA, 20 bp, artificial sequence
  Primer
gtcgtgatcgacggtcatgg

SEQ ID NO: 28 - DNA, 27 bp, artificial sequence
  Primer
ccagaccgttcagctggatattacggc

SEQ ID NO: 29 - DNA, 25 bp, artificial sequence
  Primer
aggcggccagatctgatcaagagac

SEQ ID NO: 30 - DNA, 23 bp, artificial sequence
  Primer
gttcgggacgacccgtgacattg

SEQ ID NO: 31 - DNA, 24 bp, artificial sequence
  Primer
catggcgccgacacttagcgcatc

SEQ ID NO: 32 - DNA, 660 bp, artificial sequence
  Chloramphenicol resistance gene in plasmid pUTmTn5Cm
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaa

SEQ ID NO: 33 - DNA, 3256 bp, Methylomonas sp. 16a
atgtcaatggtaatcaatacaaacatttcgtcattcaatgctcaacgccgattgaatgacacaaatatggcgatgaaaacctcaatggaaaggttgtcatcaggtttaagggtcaattccgccaaagatgacgcggcagggttggcaattggtaatgcgatgaattctcaaatcagagggatgaccgttgccgttcggaatgcgaatgacggtatttcgatggctcaaacggcggaagcggggttgggggttattaccgatactttgcaaagaatgcgggatttggccgtgcaatcggccaatagtggtgctatcacttcagacgatagggataagttgcaggcagagttcgaacagttgaacgaagaattgacacgtatcgtgacgagtaccgagttcaatggtaagcagattttggctgggtcattggcgggcggagtcaagtttcaagttggtgcgaacacagcttctgataaccaaattgcagtgcatgtcgctaatgtagccaccaccatagcatcagtgactgcagctactattgctggtgccacggcaagtgctgcaatgggggctatcggcgcgatcgacggtgcgatcaagcttatcgatactcaacgtgcccaattgggtgccattcaaaatcgctttacgacgaccatttctaatttgcaatcgtcaattgaaaatcaacaagccgcgcgttctagaataatggatgctgattttgcctctgaaactgcagcattgagtcgaaatcaaattctgcaacaagccggtgtagcgatgttggcccaagctaaccaagcgccacaaaccgtgctcagtttgttgagatagggtggtaagataacgggcgcccctttttaacgggggcacccaaagaaaagcgaggagaggtgggtcatgaataatgatatttcaaatgtcaccaggataagctttgtatcttcacccaaaatggataatgctttttccaagcaccacagtgtgaaaaataaggatattgagcaagttcaacaaatggaaaatgctcttaatataggtccagcctccgataatgaaagcaaaaccgatgataaagaggtcgataatttggtttcattagaagacgcaaaaaaacttgccgaacaaggtaataaagtttttgaagacactcagcgcaatttgcaattcaaagtagatggagagactaatcaagttgtggtgagtgtcgtagaccgaaaaagcggcgaagtcttgaggcaaataccaagtgaagaagttttggcattggccaagcgtttaaaggaattggatggagaacaagggcaaggtgtttttttgaaggaacgcgcgtaattgcacttattggaggaatgaatcatggccattacatctgcattgggcttaggaagtggcatcgatatcaacagcttggtgagccaattggtgaaagccgatggtcagccagctttaaacaccataagccgtcaagaaacgtcagtaaaatcccgattaagcgctcttggcactttgaagagcgcgttgtcgagttttcagtcggcggcggataagctgaagactgatggtctgtttagtaaacaccaggcagtgacatcgaatgaaaagattgcaacagctcaagcagggtctttggctgttgctggcagctatacactcaaagtgactcaacttgcgacctcgcataaattgatatcgggtggggctggatattccggatatggtgccgaagtaggggttggagatctgacattatccgtaggctcaaaccagttctcggtgaagatcgatggctcgaataatactttgggtggtttacgagacgcaatcaataaagctagtgacaataccggagtgaccgcaagtatcattagtgtcgatgagggctataagctcgtcttgacagccaaggaaacgggaagcgccaacaccattacgctttcatcgtccgatcctgggttgagtgatttggcgagtggcatgcaacagcagcgagcggcgctcgatgctattttcgaggtggatggccaaaccgcgactcgcagcaagaattctgtttccgacgtcatacagggtgtgacgttggagttaaagtcggaagatcccgatacggttttcgatttaaatgtttcggtcgattctaacgctattgttgaagcggcaaatgactttgtaaaggcttataacggcctgatgacgacgattaaggacttgggcaaatacgatgccgaaactcaaaagggcggcgcattggtaggtgattcgacgttgagactcattcaaagtgaaattcgtaccgaagcttctagacctgttgattcggcgggtagcaatataaattctctggcgttgatcggtatccgcattgatcaaaaagggcagatgtctttggatagttctaaatttggcgaaatagtcaagactaacctcaatgccatcggtgatgtgttttcatcggctaatggtattgccagccgattgagtagtaaattgggtgcgcatttgcaggccggcggaacactggattccagaactaaatcattgaataatcagctcaagggtttcgaagcgcggcgcgagagtgtgcaggtcaggttggataagttggagagtgcgttgttgaaacagtttattgctatggatacggcggttggacaatttcaagctaccggttcctatgtttctcaacagctggccatgcttaatcgctaaggaatctgtgagaaataagctcggcaatggtaggtgcgaattcattcgcactgtgcggataaatccgcacctacaccgaatatttgcatagtttatttcatgttcattcctaatttgataaaagagctaaagtttgtaaagaggcgaatatgagaccgagtatgtacagaaataacgtgaacaaatatgctgccgttcaaaccgatgcggcagtcgaagacgcatcacctcataaattgattcaaatgttgatgagcggttttttaatgaggatcaatgcggctaagggagcgattgagcgcggtgacttcgaggaaaaaagtattcagatatctaaagccattgcaatcgtaggtggtttgatcgatggcatcgatgccgagagaggcggtgaaatcgccaaaaatctcagaagtctttacgattacatcaatcggcagttatttgaagcaagctctaaaaacagcatcaatattttggatgaagtagctgggttgatgcgggagcttaaagaggcttgggatgcgattccagaaagttatca

SEQ ID NO: 34 - DNA, 528 bp, Methylomonas sp. 16a
tgctgtggatgattaatgttttttaatttaacgctcatgaaggcccggcatcacccaggcaaatgaaagttttctggagcgacgccttcgtcgcgacgagggcgtcgctcctacaatgactttcaaagtcagctatgctgcagggcgtattagttgtttaacgatcttgttgctgaaaatttcgtcaggtgcccccgtgtttttgatcacgacaggatcaggctgcgggcgttgactcataaacgtcttggtgtgtttatactgtaaaagaataaacgaggtcaattgaagaaagttttcgatccgggagcgatgtttggtgttaccgttcgatatcaagcaaatggtttctcaacttttgtgggagtcggtcgataaaacgactggaaatcagtgcgcgacgttggataaatgtgagtcatttcagaaaagccatcaaaatgaattaaagggatcgccactaaagccgataaatcaagcgagacaaaaataaactaacgattcgaaatccatttggaggataaggtc

SEQ ID NO: 35 - DNA, 834 bp, Methylomonas sp. 16a
  CDS (1)..(834)
atgtcaatggtaatcaatacaaacatttcgtcattcaatgctcaacgccgattgaatgacacaaatatggcgatgaaaacctcaatggaaaggttgtcatcaggtttaagggtcaattccgccaaagatgacgcggcagggttggcaattggtaatgcgatgaattctcaaatcagagggatgaccgttgccgttcggaatgcgaatgacggtatttcgatggctcaaacggcggaagcggggttgggggttattaccgatactttgcaaagaatgcgggatttggccgtgcaatcggccaatagtggtgctatcacttcagacgatagggataagttgcaggcagagttcgaacagttgaacgaagaattgacacgtatcgtgacgagtaccgagttcaatggtaagcagattttggctgggtcattggcgggcggagtcaagtttcaagttggtgcgaacacagcttctgataaccaaattgcagtgcatgtcgctaatgtagccaccaccatagcatcagtgactgcagctactattgctggtgccacggcaagtgctgcaatgggggctatcggcgcgatcgacggtgcgatcaagcttatcgatactcaacgtgcccaattgggtgccattcaaaatcgctttacgacgaccatttctaatttgcaatcgtcaattgaaaatcaacaagccgcgcgttctagaataatggatgctgattttgcctctgaaactgcagcattgagtcgaaatcaaattctgcaacaagccggtgtagcgatgttggcccaagctaaccaagcgccacaaaccgtgctcagtttgttgaga

SEQ ID NO: 36 - protein, 278 aa, Methylomonas sp. 16a
MSMVINTNISSFNAQRRLNDTNMAMKTSMERLSSGLRVNSAKDDAAGLAIGNAMNSQIRGMTVAVRNANDGISMAQTAEAGLGVITDTLQRMRDLAVQSANSGAITSDDRDKLQAEFEQLNEELTRIVTSTEFNGKQILAGSLAGGVKFQVGANTASDNQIAVHVANVATTIASVTAATIAGATASAAMGAIGAIDGAIKLIDTQRAQLGAIQNRFTTTISNLQSSIENQQAARSRIMDADFASETAALSRNQILQQAGVAMLAQANQAPQTVLSLLR

SEQ ID NO: 37 - DNA, 432 bp, Methylomonas sp. 16a
  CDS (1)..(432)
atgaataatgatatttcaaatgtcaccaggataagctttgtatcttcacccaaaatggataatgctttttccaagcaccacagtgtgaaaaataaggatattgagcaagttcaacaaatggaaaatgctcttaatataggtccagcctccgataatgaaagcaaaaccgatgataaagaggtcgataatttggtttcattagaagacgcaaaaaaacttgccgaacaaggtaataaagtttttgaagacactcagcgcaatttgcaattcaaagtagatggagagactaatcaagttgtggtgagtgtcgtagaccgaaaaagcggcgaagtcttgaggcaaataccaagtgaagaagttttggcattggccaagcgtttaaaggaattggatggagaacaagggcaaggtgtttttttgaaggaacgcgcg

SEQ ID NO: 38 - protein, 144 aa, Methylomonas sp. 16a
MNNDISNVTRISFVSSPKMDNAFSKHHSVKNKDIEQVQQMENALNIGPASDNESKTDDKEVDNLVSLEDAKKLAEQGNKVFEDTQRNLQFKVDGETNQVVVSVVDRKSGEVLRQIPSEEVLALAKRLKELDGEQGQGVFLKERA

SEQ ID NO: 39 - DNA, 1347 bp, Methylomonas sp. 16a
  CDS (1)..(1347)
atggccattacatctgcattgggcttaggaagtggcatcgatatcaacagcttggtgagccaattggtgaaagccgatggtcagccagctttaaacaccataagccgtcaagaaacgtcagtaaaatcccgattaagcgctcttggcactttgaagagcgcgttgtcgagttttcagtcggcggcggataagctgaagactgatggtctgtttagtaaacaccaggcagtgacatcgaatgaaaagattgcaacagctcaagcagggtctttggctgttgctggcagctatacactcaaagtgactcaacttgcgacctcgcataaattgatatcgggtggggctggatattccggatatggtgccgaagtaggggttggagatctgacattatccgtaggctcaaaccagttctcggtgaagatcgatggctcgaataatactttgggtggtttacgagacgcaatcaataaagctagtgacaataccggagtgaccgcaagtatcattagtgtcgatgagggctataagctcgtcttgacagccaaggaaacgggaagcgccaacaccattacgctttcatcgtccgatcctgggttgagtgatttggcgagtggcatgcaacagcagcgagcggcgctcgatgctattttcgaggtggatggccaaaccgcgactcgcagcaagaattctgtttccgacgtcatacagggtgtgacgttggagttaaagtcggaagatcccgatacggttttcgatttaaatgtttcggtcgattctaacgctattgttgaagcggcaaatgactttgtaaaggcttataacggcctgatgacgacgattaaggacttgggcaaatacgatgccgaaactcaaaagggcggcgcattggtaggtgattcgacgttgagactcattcaaagtgaaattcgtaccgaagcttctagacctgttgattcggcgggtagcaatataaattctctggcgttgatcggtatccgcattgatcaaaaagggcagatgtctttggatagttctaaatttggcgaaatagtcaagactaacctcaatgccatcggtgatgtgttttcatcggctaatggtattgccagccgattgagtagtaaattgggtgcgcatttgcaggccggcggaacactggattccagaactaaatcattgaataatcagctcaagggtttcgaagcgcggcgcgagagtgtgcaggtcaggttggataagttggagagtgcgttgttgaaacagtttattgctatggatacggcggttggacaatttcaagctaccggttcctatgtttctcaacagctggccatgcttaatcgc

SEQ ID NO: 40 - protein, 449 aa, Methylomonas sp. 16a
MAITSALGLGSGIDINSLVSQLVKADGQPALNTISRQETSVKSRLSALGTLKSALSSFQSAADKLKTDGLFSKHQAVTSNEKIATAQAGSLAVAGSYTLKVTQLATSHKLISGGAGYSGYGAEVGVGDLTLSVGSNQFSVKIDGSNNTLGGLRDAINKASDNTGVTASIISVDEGYKLVLTAKETGSANTITLSSSDPGLSDLASGMQQQRAALDAIFEVDGQTATRSKNSVSDVIQGVTLELKSEDPDTVFDLNVSVDSNAIVEAANDFVKAYNGLMTTIKDLGKYDAETQKGGALVGDSTLRLIQSEIRTEASRPVDSAGSNINSLALIGIRIDQKGQMSLDSSKFGEIVKTNLNAIGDVFSSANGIASRLSSKLGAHLQAGGTLDSRTKSLNNQLKGFEARRESVQVRLDKLESALLKQFIAMDTAVGQFQATGSYVSQQLAMLNR

SEQ ID NO: 41 - DNA, 396 bp, Methylomonas sp. 16a
  CDS (1)..(396)
atgagaccgagtatgtacagaaataacgtgaacaaatatgctgccgttcaaaccgatgcggcagtcgaagacgcatcacctcataaattgattcaaatgttgatgagcggttttttaatgaggatcaatgcggctaagggagcgattgagcgcggtgacttcgaggaaaaaagtattcagatatctaaagccattgcaatcgtaggtggtttgatcgatggcatcgatgccgagagaggcggtgaaatcgccaaaaatctcagaagtctttacgattacatcaatcggcagttatttgaagcaagctctaaaaacagcatcaatattttggatgaagtagctgggttgatgcgggagcttaaagaggcttgggatgcgattccagaaagttatcaa

SEQ ID NO: 42 - protein, 132 aa, Methylomonas sp. 16a
MRPSMYRNNVNKYAAVQTDAAVEDASPHKLIQMLMSGFLMRINAAKGAIERGDFEEKSIQISKAIAIVGGLIDGIDAERGGEIAKNLRSLYDYINRQLFEASSKNSINILDEVAGLMRELKEAWDAIPESYQ

SEQ ID NO: 43 - DNA, 1429 bp, Methylomonas sp. 16a
cggtatgcttaacacatgcaagtcgaacgctgaagggtgcttgcacctggatgagtggcggacgggtgagtaatgcataggaatctgcctattagtgggggataacgtggggaaactcacgctaataccgcatacgctctacggaggaaagccggggaccttcgggcctggcgctaatagatgagcctatgtcggattagctagttggtggggtaaaggcctaccaaggcgacgatccgtagctggtctgagaggatgatcagccacactgggactgagacacggcccagactcctacgggaggcagcagtggggaatattggacaatgggcgcaagcctgatccagcaataccgcgtgtgtgaagaaggcctgagggttgtaaagcactttcaatgggaaggaacacctatcggttaatacccggtagactgacattacccatacaagaagcaccggctaactccgtgccagcagccgcggtaatacggagggtgcaagcgttaatcggaattactgggcgtaaagcgtgcgtaggcggttttttaagtcagatgtgaaagccctgggcttaacctgggaactgcatttgatactggggaactagagttgagtagaggagagtggaatttcaggtgtagcggtgaaatgcgtagagatctgaaggaacaccagtggcgaaggcggctctctggactcaaactgacgctgaggtacgaaagcgtgggtagcaaacaggattagataccctggtagtccacgccgtaaacgatgtcaactaaccgttgggttcttaaagaacttagtggtggagctaacgtattaagttgaccgcctggggagtacggccgcaaggctaaaactcaaatgaattgacgggggcccgcacaagcggtggagcatgtggtttaattcgatgcaacgcgaagaaccttacctacccttgacatcctcggaacttgtcagagatgacttggtgccttcgggaaccgagagacaggtgctgcatggctgtcgtcagctcgtgtcgtgagatgttgggttaagtcccgtaacgagcgcaacccttatccttagttgccagcgcgtcatggcgggaactctagggagactgccggtgataaaccggaggaaggtggggacgacgtcaagtcatcatggcccttatgggtagggctacacacgtgctacaatggtcggtacagagggttgcgaactcgcgagagccagccaatcccaaaaagccgatcctagtccggattgcagtctgcaactcgacttgcatgaagtcggaatcgctagtaatcgcggatcagaatgccgcggtgaatacgttcccgggccttgtacacaccgcccgtcacaccatgggagtgggttgcaaaagaagtaggtagtttaaccttcgggagggcgcttaccactttgtg